(19) United States
(12) Patent Application Publication
(10) Pub. No.: US 2023/0107066 A1
(43) Pub. Date: Apr. 6, 2023

(54) MICROELECTRONIC SENSORS FOR DETECTION OF ANALYTES, DEVICES AND METHODS USING THE SAME

(71) Applicant: RAM MEDICAL CORPORATION L.L.C., Dubai (AE)

(72) Inventors: Ayal RAM, Singapore (SG); Walid-Madhat MUNIEF, Blieskastel (DE); Srinivas GANTI, Zweibrucken (DE); Nikhil KARUNAKARAN PONON, Ramstein-Miesenbach (DE)

(21) Appl. No.: 17/950,893

(22) Filed: Sep. 22, 2022

Related U.S. Application Data
(63) Continuation of application No. PCT/IB2021/052356, filed on Mar. 22, 2021.
(60) Provisional application No. 63/124,116, filed on Dec. 11, 2020.
(60) Provisional application No. 63/063,524, filed on Aug. 10, 2020.
(60) Provisional application No. 63/063,521, filed on Aug. 10, 2020.
(60) Provisional application No. 63/003,331, filed on Apr. 1, 2020.
(60) Provisional application No. 62/993,832, filed on Mar. 24, 2020.

(51) Int. Cl. G01N 33/497

(52) U.S. Cl. CPC G01N 33/497

(57) ABSTRACT

A microelectronic sensor for non-invasive and label-free chemical detection and biomolecular diagnostics of analytes in a raw sample (without pre-treatment and without purification) is described in the present invention. The sensor comprises a microelectronic chip and a sample collection system attached to said microelectronic chip or incorporating said microelectronic chip. The sample collection system may be a sampling swab attached to the microelectronic chip or a breathalyser tube incorporating the microelectronic chip. The microelectronic chip contains a nanoarray of metamolecules configured to detect and transmit signals through the sample in a THz frequency range, and an integrated circuit for storing and processing signals in a THz frequency domain, and for modulating and demodulating radio-frequency (RF) signals. The metamolecules are composed of split-ring resonators and a wave container or a wave bouncer confining or bouncing waves received from the split-ring resonators, and further exciting a dark mode in the split-ring resonators.

### CROSS REFERENCE TO RELATED APPLICATIONS

This application is a Bypass Continuation of PCT Patent Application No. PCT/IB2021/052356 having International filing date of Mar. 22, 2021 which claims the benefit of priority of U.S. Provisional Patent Application Nos. 62/993,832, filed on Mar. 24, 2020, 63/003,331, filed on Apr. 1, 2020, 63/063,521, filed on Aug. 10, 2020, 63/063,524, filed on Aug. 10, 2020 and 63/124,116, filed on Dec. 11, 2020, the contents of which are all incorporated herein by reference in their entirety.

### TECHNICAL FIELD

The present application relates to the field of microelectronic devices used in detection and continuous monitoring of electrical signals in a terahertz (THz) frequency range. In particular, the present application relates to a microelectronic sensor comprising a terahertz (THz) nanoantenna structure arranged in a periodic array for non-invasive, label-free and on-site chemical sensing and biomolecular diagnostics and detection of analytes.

### BACKGROUND

### Chemical Sensing and Biomolecular Diagnostics

Chemical sensing is likely the most primordial sensory modality that emerged in the evolution of life. Without chemical sensing life on earth would probably not exist. It is used for detecting nutrients, avoiding threats, finding mating partners and various forms of communication and social interaction between animals.

The advent of artificial sensors has created a myriad of problems in the areas of chemical detection and identification with applications in food quality and pollution control, chemical threat detection, health monitoring, robot control and even odour and taste synthesis. Efficient algorithms are needed to address many challenges of chemical sensing in these areas, including (but not limited to) sensitivity levels, sensor drift, concentration invariance of analyte identity and complex mixtures.

As an example, biological pathogens, including biological threat agents, are living organisms that reproduce and sustain a population, which amplify, grow and re-infect, thereby resulting in an epidemic situation. The biological pathogens represent an extremely diverse range of microorganisms, which have no seemingly common attributes other than infecting the human and animal populations. The problem is therefore to detect and identify them at the earliest stage of invasion and at the lowest concentration.

Prior to DNA sequencing, the highest resolution techniques provided only protein and peptide-level structures as targets of analysis and assays. Many of the well-established protocols called for the examination of the size and shape of the pathogens along with the examination of the expressed proteins through biochemical and immunochemical assays. Advances in DNA sequencing technology have made it possible for scientists all over the world to sequence complete microbial genomes rapidly and efficiently. Access to the DNA sequences of entire microbial genomes has recently offered new opportunities to analyse and understand pathogens at the molecular level. Modern DNA sequencing techniques are able to detect pathogens in biological tissues and study variations in gene expression in response to the pathogenic invasion. These responses help in designing novel approaches for microbial pathogen detection and drug development. Identification of certain microbial pathogens as etiologic agents responsible for chronic diseases is leading to new treatments and prevention strategies for these diseases.

Majority of the modern chemical sensors used in pathogen detection are based upon the sequence-based recognition of DNA, structural recognition of pathogens or pathogen biomarkers, or cell-based function. However, the selection of the pathogen biomarkers introduces a serious challenge in the development of the sensors for detection of the biological pathogens. This is because most of the pathogen biomarkers have low selectivity and can distinguish between general classes of microorganisms, but are not able to identify the specific species or strain of organism. For example, calcium dipicolinate is a unique component of endospores. Dipicolinic acid can therefore be used to indicate the presence of endospores, but it cannot be able to distinguish between very dangerous Bacillus anthracis spores and other non-toxic Bacillus spores. The presence of the DNA as an additional indicator will be able to determine that the unknown material is biological in nature but will not be able to identify its source (unless extensive sequence-based analysis is used). Also, cell metabolites are generally common to many different cell types and therefore extremely difficult to use for discrimination between specific microorganisms. In view of the above, there is a long-felt need for new methods and devices to detect and identify biological pathogens.

The use of the ultrasensitive and highly selective microelectronic sensors for the biological pathogen detection is the area that has not been developed yet. The reasons for that are many. Sensor arrays that detect multiple pathogen biomarkers produce a large number of false alarms because of their low selectivity. The concept of sensor arrays has been successfully used in the field of vapour analysis. In this approach each particular sensor of the sensor array was designed to respond to different properties of the vapours, followed by statistical methods to specifically identify the particular vapour from the fingerprint of the generated response from all the sensors of the array. However, since each pathogen species carries with it a unique DNA or RNA signature that differentiate it from other organisms, such approach cannot be effectively used for pathogen detection. In other words, each sensor of the array responds to different properties (biomarkers) of a pathogen. Therefore, such approach would require a well-characterised and already identified background signal to determine the fingerprints that would constitute a positive signal.

The ideal solution for a real-time sensing would be any specific response of a biological organism that results in instantaneous, specific and repeatable identification. However, as noted above, there are considerable technological and practical difficulties in the development of sensors that provide a real-time response for all three of these criteria. Immunoassay techniques might give a similar specific analysis. However, their drawback, other than the long response time, is the requirement for special chemical consumables that add considerably to the logistic burden and costs. These can increase operational costs by hundreds of dollars per hour.

Optical technologies intrinsically result in real-time biochemical detection. Sensors based on these technologies have been available to military and civil defence for quite some time. However, the common drawback of the optical sensors is low specificity. The sensors mostly offer a generic detection capability at best, since the optical similarity of the target particles with benign, naturally occurring backgrounds makes them difficult to distinguish. There are the some of the currently employed bio-agent detections strategies. Most represent a compromise between specificity, speed and cost.

Quantitative Polymerase Chain Reaction (qPCR) is capable of amplification and detection of a DNA sample from a single bio-agent cell within 30 minutes. Knowing the pathogen nucleic acid sequence makes it possible to construct oligoes for pathogen detection. These oligoes are at the basis of many highly specific analytical tests now on the market.

Microarray-based detection can combine powerful nucleic acid amplification strategies with the massive screening capability of microarray technology, resulting in a high level of sensitivity, specificity, and throughput. In addition to the previously mentioned caveats, the cost and organizational complexity of performing a large number of PCR reactions for downstream microarray applications render this option feasible but unattractive. This limitation has severely reduced the utility of this technique and impeded the continued development of downstream applications.

To sum up, the problem of accurate and reliable identification of pathogenic agents and their corresponding diseases is the weakest point in biological agent detection capability today. There is intense research for new molecular detection technologies that could be used for very accurate detection of pathogens that would be a concern to first responders. These include the need for ultrasensitive and highly selective sensors for biological pathogens detection in environmental, forensic and military applications. The benefits of specific (accurate) detection include saving millions of dollars annually by reducing disruption of the workforce and the national economy and improving delivery of correct protective countermeasures.

All said above regarding detection of biological pathogens also relate to the detection of other chemical and biological compounds, which may present threat or have medical reasons to be detected. The examples are many and may include explosives, toxins, DNA, proteins etc.

### THz Spectroscopy

Recently it has become clear to scientists that terahertz (THz) radiation could be extremely important for research related to the life sciences because of the unique capability of these low energy electromagnetic waves to interact with vibrations of atoms within biological molecules to produce specific molecular fingerprints (see for example, Globus et al. in “Terahertz Fourier transform characterization of biological materials in a liquidphase”, J. Physics D: Applied Physics, 39(15), 3405-3413). THz spectroscopy uses wavelengths beyond those traditionally used for chemical and biomolecular analysis. Biological materials have found to be active in the frequency range of 30 GHz to 300 THz (the wavelength range, about 1 cm to 1 μm). These frequency and wavelength domains, the spectral range between the upper end of the radio frequencies and microwaves and the lowest optical frequencies were named the ‘Terahertz Gap’, because so little was known about them and because of the absence of radiation sources and detectors.

THz vibrational spectroscopy is entirely based on the interaction of radiation in this particular frequency range with internal molecular vibrations of low energy. A majority of the THz experimental data have recently been reported on frequencies in this range and for relatively small biological molecules that are often prepared in crystalline form (for example, Heilweil et al. (2008), “Terahertz Spectroscopy of Biomolecules”, In Terahertz Spectroscopy, Taylor and Francis, London, 2008, Chapter 7, pp 269-297). Low-energy THz radiation interacts with the low-frequency internal molecular motions (vibrations) involving the weakest hydrogen bonds (H-bonds) and other weak connections within molecules by exciting these vibrations. The width of individual spectral lines and the intensity of resonance features, which are observed in THz spectroscopy, are very sensitive to the relaxation processes of atomic dynamics (displacements) within a molecule. Those relaxation processes determine the discriminative capabilities of THz spectroscopy. Appropriate spectral resolution must be used in THz spectroscopy to be able to acquire qualitative as well as quantitative information used to identify the molecules that will, in turn, increase detection accuracy and selectivity. Because of their very small size and relatively low absorption coefficient, the waves of the THz radiation easily propagate through any liquid, such as water, serum or any biological medium including the entire biological object, for example cells and skin.

U.S. Pat. No. 10,502,665 describes an aerosol collection macrosystem for collecting and analysing bioaerosols, including exhaled breath aerosol from a subject. This aerosol collection system is used for routine analysis of breath samples using analytical techniques, such as SDS-PAGE-based protein colorimetric assay followed by LCMS/MS analysis. Kazunori Serita et al., “A terahertz-microfluidic chip with a few arrays of asymmetric meta-atoms for the ultra-trace sensing of solutions”, Photonics 2019, 6(1), 12, teaches a non-linear optical crystal-based THz-microfluidic chip with a few arrays of asymmetrical meta-atoms, an elementary unit of metamaterials, for measuring ultra-trace amounts of solution samples. By optimizing the structural asymmetry, as well as the periods and numbers of arrayed meta-atoms, sharp Fano resonances were observed when the central meta-atom was irradiated by localised THz waves.

### SUMMARY

The present invention describes embodiments of a microelectronic sensor for non-invasive and label-free chemical detection and biomolecular diagnostics of analytes in a raw sample (without pre-treatment and without purification), comprising a microelectronic chip and a sample collection system attached to said microelectronic chip or incorporating said microelectronic chip, said sample collection system is suitable for sample collection of a raw sample taken directly from a subject being tested without any purification and without any chemical or biological separation, and for delivery of the sample to said microelectronic chip, wherein said microelectronic chip comprises:
(1) a nanoantenna structure, said nanoantenna structure is arranged in a periodic array of metamolecules and configured to detect and transmit signals through said sample in a terahertz (THz) frequency range; and
(2) an integrated circuit for storing and processing signals in a THz frequency domain, and for modulating and demodulating radio-frequency (RF) signals;
characterised in that each of said metamolecules in the array is composed of at least one split-ring resonator and a wave container or a wave bouncer, said wave container confines and said wave bouncer bounces electromagnetic waves received from said at least one split-ring resonator, both the wave container and the wave bouncer are designed to excite a dark mode in said at least one split-ring resonator, followed by coupling the excited dark mode back into said at least one split-ring resonator.

In some embodiments, the split-ring resonators are composed of a square, round, rectangular, hexagonal, spiral or any other shape ring (wire) having at least one split (gap) in the ring and suitable for resonating in the THz frequency range. In other embodiments, the split-ring resonators are asymmetric. In particular embodiments, the split-ring resonators have a geometry or shape selected from rod split-rings, round (circular) split-rings, square-split rings, rectangular split-rings, hexagonal split-rings, nested split-rings, single split-rings, split-rings having more than one split in their ring, deformed split-rings, spiral split-rings and spiral resonators suitable for resonating in the THz frequency range.

In other embodiments, the wave container is selected from a metal ring (circle), metal square, metal rectangle, metal hexagon and any other shape or array thereof suitable for confining electromagnetic waves received from said at least one split-ring resonator, said wave container is designed to excite a dark mode in said at least one split-ring resonator, followed by coupling the excited dark mode back into said at least one split-ring resonator. In still other embodiments, the wave bouncer is selected from a metal bar, metal segment or any other metal fragment or array thereof suitable for bouncing electromagnetic waves received from said at least one split-ring resonator, said wave bouncer is designed to excite a dark mode in said at least one split-ring resonator, followed by coupling the excited dark mode back into said at least one split-ring resonator.

In a specific embodiment, each metamolecule is composed of two square-shape split-ring resonators and a single metal bar over the resonators, said metal bar is designed to excite a dark mode in said resonators, followed by coupling it into the resonators. In other specific embodiments, each metamolecule is composed of a spiral-shape resonator and a metal ring container surrounding and confining said spiral-shape resonator, said metal ring is designed to excite a dark mode in the spiral-shape resonator, followed by coupling it into the said spiral-shape resonator. In a particular embodiment, each metamolecule is composed of a round (circular)-shape split-ring resonator having at least two splits in the ring and a metal bar under said round-shape split-ring resonator, said metal bar is designed to excite a dark mode in the round-shape split-ring resonator, followed by coupling it into said split-ring resonator. In a further specific embodiment, each metamolecule is composed of a hexagon-shape split-ring resonator having at least one split in the ring and six metal hexagons surrounding said hexagon-shape split-ring resonator, said metal hexagons are designed to excite a dark mode in the (inner) hexagon-shape split-ring resonator, followed by coupling it into said hexagon-shape split-ring resonator. In yet further specific embodiment, each metamolecule is composed of a round (circular)-shape split-ring resonator having at least two splits in the ring and a metal square-shaped container (box), said metal square-shaped container is designed to excite a dark mode in the round-shape split-ring resonator, followed by coupling it into said split-ring resonator. Walls of this metal square-shaped wave container can be optionally symmetrically split to form additional resonance structures creating the bright mode. Since these splits are symmetrical, they can be used as a reference by rotating the chip.

In a further embodiment, the sample collection system is a sampling swab attached to the microelectronic chip. In yet further embodiment, the sample collection system is a breathalyser tube incorporating the microelectronic chip. In still another embodiment, the microelectronic senor of the present invention is inserted in a laboratory THz spectrometer for laboratory measurements.

In one embodiment, the integrated circuit of the microelectronic sensor of the present invention further comprises:
(a) an μ-pulse generator for pulsed RF signal generation;
(b) an integrated DC-RF current amplifier or lock-in amplifier connected to said μ-pulse generator for amplification of the signal obtained from said μ-pulse generator;
(c) an analogue-to-digital converter (ADC) with in-built digital input/output card connected to the amplifier for converting the received analogue signal to a digital signal and outputting said digital signal to a microcontroller unit;
(d) the microcontroller unit (MCU) for processing and converting the received digital signal into data readable in a user interface or external memory; and
(e) a wireless connection module for wireless connection of said breathalyser to said user interface or external memory.

In another embodiment, the integrated circuit of the microelectronic sensor of the present invention further comprises:
(1) one or two out-input RFID-tag zero-power fractal antennas, each connected to the circuit, for RFID-tagging and further tracking a particular individual;
(2) a diode input-output separator to separate polarities in said circuit;
(3) an RFID integrated circuit for storing and processing signals received from said individual, and for modulating and demodulating radio-frequency (RF) signals, said RFID integrated circuit comprising:
   (a) a voltage source supplying electric current to said breathalyser and to said one or two RFID-tag zero-power fractal antennas;
   (b) an integrated or CMOS current amplifier for amplification of an electric current obtained from said breathalyser;
   (c) an analogue-to-digital converter (ADC) with wireless input/output modules connected to said current amplifier for wireless outputting the converted signal to a user interface or external memory;
   (d) a microcontroller unit (MCU) for processing and converting the received signal into data readable in said user interface or external memory; and
   (e) a wireless connection module for wireless connecting of said sensor to said user interface or external memory.

In some embodiments, the external memory is selected from a mobile device, such as a smartphone or smart watch, desktop computer, server, remote storage, internet storage or cloud. The substrate is composed of glass, silicon or quartz.

The present application describes embodiments of a breathalyser for non-invasive and label-free chemical detection and biomolecular diagnostics of raw breath sample received directly from a subject being tested without any substantive purification and without any chemical or biological separation, comprising:

an integrated tube having an exhalation portion with an inlet (air intake) area and an exhaust portion with an outlet (focusing) area, said tube being placed in a housing transparent to terahertz radiation and suitable for collecting a sample of exhalation air and transferring said sample to a testing chamber;

the testing chamber integrated inside said housing, attached to said exhalation portion and designed to provide housing for an integrated circuit, battery and other electronic components, and to receive, filter and analyse said sample, said testing chamber comprises at least one filter suitable for filtering the sample and the integrated microelectronic sensor; and an integrated circuit for storing and processing signals in a THz (terahertz) frequency domain, and for modulating and demodulating radio-frequency (RF) signals;
wherein said microelectronic sensor comprises a microelectronic chip and a sample collection system attached to said microelectronic chip or incorporating said microelectronic chip, said sample collection system is suitable for sample collection of a raw sample taken directly from a subject being tested without any purification and without any chemical or biological separation, and for delivery of the sample to said microelectronic chip, wherein said microelectronic chip comprises:
(1) a nanoantenna structure, said nanoantenna structure is arranged in a periodic array of metamolecules and configured to detect and transmit signals through said sample in a terahertz (THz) frequency range; and
(2) an integrated circuit for storing and processing signals in a THz frequency domain, and for modulating and demodulating radio-frequency (RF) signals;
characterised in that each of said metamolecules in the array is composed of at least one split-ring resonator and a wave container or a wave bouncer, said wave container confines and said wave bouncer bounces electromagnetic waves received from said at least one split-ring resonator, both the wave container and the wave bouncer are designed to excite a dark mode in said at least one split-ring resonator, followed by coupling the excited dark mode back into said at least one split-ring resonator.

The exhalation portion of the breathalyser of the embodiments serves for blowing air (breath) containing an analyte to be tested onto the microelectronic chip installed inside the testing chamber. The breathalyser further contains a disposable adapter attached to the exhalation portion and suitable for receiving the exhalation air and transferring it to the exhalation portion of the breathalyser. The collected sample of the blown (exhalation) air flows through the exhalation portion to the testing chamber, is filtered there by passing through at least one suitable air filter, followed by projecting on the microelectronic chip of the present invention. This way, the aerosol droplets and water vapours carrying bio-molecules and viruses, as well as other airborne particles in the exhaled breath can be collected as micro-droplets on the sensing nanoantenna structure.

The exhalation portion, testing chamber and their dimensions are fully customizable and determine the amount and particle size of the aerosol particles collected from breath. The testing chamber is designed in such a way that it allows the droplets to dry in a very short period of time, less than 60 seconds. The breathalyser of the present embodiments is completely transparent to THz radiation.

The exhalation portion of the breathalyser optionally has a soft membrane or mechanical valve which is designed to move based on the air pressure to ensure that there is a sufficient amount of the blown air collected for measurements (a user has blown hard enough into the breathalyser and the proper amount of breath particles and vapours have been collected). The breathalyser has optionally a teeth grip ring for easy use and firm holding, so that when blowing, it does not pop out of the user's mount. The breathalyser of the present invention is a single-use device and can be operated entirely by the user with no help from medical personnel. Optionally, it can be integrated into a medical setting and used by medical personnel.

In one embodiment, the nanoantenna periodic structure is composed of resonant circuit elements capable of resonating in the THz range. These resonant circuit elements act as electrical resonators storing energy oscillating at the circuit's resonant frequency. They are composed of inductor-capacitor (LC) electric circuits having nano-gaps in the structure and capable of measuring capacitance of the sample. An example of such nanoantenna periodic structure is an Aharonov-Bohm antenna. The nanoantenna periodic structure of the embodiments is selectively composed of gold, gold/chromium, gold/doped silicon/silver or other similar metal periodic structures.

In another embodiment, the nanoantenna periodic structure is composed of metamaterials such as graphene, graphene/gold or copper/single layer graphene/copper composite, thereby creating metasurfaces, which are thin two-dimensional metamaterial layers that modulate (allow or inhibit) propagation of electromagnetic waves in desired directions. In some embodiments, the nanoantenna periodic structure further comprises metallic nanoparticles, such as gold nanoparticles deposited on said periodic structure, to create plasmonic effects upon irradiation of the structure with excitation light. In still another embodiment, the nanoantenna periodic structure further comprises an electro-optical crystal (EOC) transducer layer, such as LiNbO₃, deposited on said periodic structure and designed to be brought into a contact with the sample and illuminated with a polarised light, thereby making it suitable to modulate the structure capacitance and inductance, and increase sensitivity of the sensor. The breathalyser of these embodiments, further comprises a modulated light source, such as a surface-mounted-device light-emitting diode (SMD LED) or UV—VIS-IR laser diode, for irradiating such plasmonic or electro-optic periodic structure.

In a certain embodiment, the nanoantenna periodic structure further comprises at least one chemical or biomolecular layer immobilised on top of said nanoantenna periodic structure and capable of binding or adsorbing analytes being tested from the sample. In a specific embodiment, the chemical or biomolecular layer is a cyclodextrin, 2,2,3,3-tetrafluoropropyloxy-substituted phthalocyanine or their derivatives, or said chemical or biomolecular layer comprises capturing biological molecules, such as primary, secondary antibodies or fragments thereof against certain proteins to be detected, or their corresponding antigens, enzymes or their substrates, short peptides, specific polynucleotide sequences, which are complimentary to the sequences of DNA to be detected, aptamers, receptor proteins or molecularly imprinted polymers.

In a further embodiment, a method for label-free chemical detection and biomolecular diagnostics using the breathalyser of the present embodiments comprises:
(a) Blowing an air into the exhalation portion of the breathalyser of the present invention;
(b) Recording electrical signals received from the breathalyser over time at a resonance frequency in the THz frequency domain, said resonance frequency is dependent on inductance and capacitance of an analyte being tested in the sample and pre-selected based on a calibration of the sensor for said analyte;
(c) Transmitting the recorded signals from said breathalyser to an external memory for further processing; and
(d) Converting the transmitted signals to digital signals and processing the digital signals in the external memory in a form of frequency waveforms, comparing the recorded frequency waveforms with negative control waveforms stored in the external memory, and extracting chemical and biomolecular information from said waveforms in a form of readable data, thereby detecting and/or identifying a particular analyte in the sample.

In a particular embodiment, each of said analytes being tested is characterised by a distinguished shift in the resonant frequency and by a unique fingerprint area in the recorded frequency waveform. In another particular embodiment, the analyte is selected from the group of:
toxic metals, such as chromium, cadmium or lead,
regulated ozone-depleting chlorinated hydrocarbons,
food toxins, such as aflatoxin, and shellfish poisoning toxins, such as saxitoxin or microcystin,
neurotoxic compounds, such as methanol, manganese glutamate, nitrix oxide, tetanus toxin or tetrodotoxin, Botox, oxybenzone, Bisphenol A, or butylated hydroxyanisole,
explosives, such as picrates, nitrates, trinitro derivatives, such as 2,4,6-trinitrotoluene (TNT), 1,3,5-trinitro-1,3,5-triazinane (RDX), trinitroglycerine, pentaerythritol tetranitrate (PETN), N-methyl-N-(2,4,6-trinitrophenyl)nitramide (nitramine or tetryl), nitric ester, azide, derivatives of chloric and perchloric acids, fulminate, acetylide, and nitrogen rich compounds, such as tetrazene, octahydro-1,3,5,7-tetranitro-1,3,5,7-tetrazocine (HMX), peroxide, such as triacetone trioxide, C4 plastic explosive and ozonidesor, or an associated compound of said explosives, such as a decomposition gases or taggants, and
biological pathogens, such as a respiratory viral or bacterial pathogen, an airborne pathogen, a plant pathogen, a pathogen from infected animals or a human viral pathogen.

In a specific embodiment, the viral pathogen detected in the method of the present invention is SARS-CoV-2.

Various embodiments may allow various benefits and may be used in conjunction with various applications. The details of one or more embodiments are set forth in the accompanying figures and the description below. Other features, objects and advantages of the described techniques will be apparent from the description and drawings and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Disclosed embodiments will be understood and appreciated more fully from the following detailed description taken in conjunction with the appended figures. The drawings included and described herein are schematic and are not limiting the scope of the disclosure. It is also noted that in the drawings, the size of some elements may be exaggerated and, therefore, not drawn to scale for illustrative purposes. The dimensions and the relative dimensions do not necessarily correspond to actual reductions to practice of the disclosure.

FIGS. 1a-1b schematically show a resonant circuit element of the nanoantenna structure used for sensing pathogens, such as viruses.

FIG. 1c schematically shows a fragment of the nanoantenna structure of the present invention, deposited on the substrate, arranged in a periodic array and configured to detect and transmit signals through the sample in a terahertz (THz) frequency range.

FIG. 1d schematically shows the detection process of a viral pathogen with the nanoantenna structure of the present invention.

FIG. 2a demonstrates an exemplary generated spectral signal and FIG. 2b shows the corresponding Laplacian wavelet representation. The wavelet scale on the y-axis represents the width-scale parameter (a), and the x-axis represents the wavelet shift (b).

FIG. 3 shows resonant frequency accuracy comparison when using peak-find vs wavelet.

FIG. 4 shows reference transform with a user-defined region of interest (red bar) and the measured maximum value (red dot).

FIGS. 5a-5b show feature classification of labelled samples based on the sensor antenna resonance shifts.

FIG. 6 shows dependence of resonance frequency shift on CT (cycle threshold) value of the PCR test.

FIG. 7a shows a relative intensity of the signal in a frequency range of interest between 0.9 THz and 1.1 THz corresponding to the resonance frequency of the virus (shaded area).

FIG. 7b spectra in the frequency range 0.90-1.08 THz, from blind tests that fall within the standard deviation of control.

FIG. 8a shows an exemplary wavelet transform for SB_30 positive (+ve) blind test.

FIG. 8b shows the absolute classification of positive and negative samples, including the prediction for unknown-blind and randomised samples.

FIG. 9 shows feature importance extracted using the AdaBoost algorithm.

FIG. 10a shows a reference spectrum taken with a TDS-THz spectrum analysis software.

FIG. 10b shows the FFT of the reference measurement shown in FIG. 10a.

FIG. 11a shows the raw FFT transmission power spectra for different Anti-Cardiac Troponin I antibody (antiCTn1) concentrations.

FIG. 11b shows the expanded FFT transmission power spectra around the resonance peak for different Anti-Cardiac Troponin I antibody (antiCTn1) concentrations.

FIG. 11c shows impulse response for the different antibody concentrations.

FIG. 11d shows the expansion of the spectra of FIG. 11c for visualisation of the resonance peak.

FIGS. 12a and 12b show the transmission power spectrum and its expansion around the resonance peak, respectively, for 2 mg/ml antiCTn1 (cyan line) and the mixture of 2 mg/ml antiCTn1 (green line) with troponin.

FIGS. 13a-13f show the test results with the split-ring resonators nanoantenna as a sensing element operating in a pure LC mode on the SARS-CoV-2 infected samples versus healthy patient samples.

FIG. 13a shows a plot of the THz FFT spectra collected for all positive (dark blue and green colours) and negative (light blue and green colours) samples, with two different (blue and green) positive and negative samples, shown in dark and light colour schemes respectively.

FIG. 13b shows the expansion of the spectra of FIG. 13a for better visualisation.

FIG. 13c shows the zoomed region in the THz FFT spectrum of the specific sample SB_27, where the main region of difference for the positive versus negative samples is indicated with the circle.

FIGS. 13d-13f show the zoomed peaks from FIG. 13b for three selected samples, where the circle highlights the main region of difference for the positive versus negative samples.

FIGS. 14a-14b show the exemplary cross correlogram maps demonstrating the correlation between the sensor with sample and sensor without any sample (reference) with a slicing window of 600 GHz moved over the entire frequency range. Axis X is a spectral band (THz), and axis Y is a frequency shift (THz).

FIG. 15a shows the normalised cross-correlogram difference map between positive and negative COVID-sample spectra taken against the reference. This is a rescaled DELTA plot obtained from FIGS. 14a-14b with a distinct band in the resonance region 1.2-1.3 THz and also around 2.6-2.9 THz, the latter corresponds to the absorption lines of water.

FIG. 15b shows the DELTA cross-correlogram between positive and negative samples at frequency between 2.4-2.9 THz.

FIG. 16a shows the metamolecule of the metamaterial of the present invention, supporting toroidal dipolar excitation with incorporated silicon strips. The exemplary diameter of the metamolecule is 30 μm with the central gap equal to 2 μm and the lateral gaps 5 μm. The silicon strips have the gaps 1.4 μm.

FIG. 16b shows the metamolecule as a fragment of the metamaterial of the present invention, supporting toroidal dipolar excitation. Red arrows show displacement currents j induced by the vertically polarized plane wave, blue arrow shows toroidal dipole moments T of the metamolecule, while green arrow shows circulated magnetic moment m (Courtesy of Basharin (2017)).

FIG. 16c shows the amplitude of the conductive currents j induced in the metamolecule at approximately 4.8 THz frequency (Courtesy of Basharin (2017)).

FIG. 16d shows the photographic image of the toroidal metamaterial of the present invention (Courtesy of Basharin (2017)).

FIGS. 17a-17b show an example of the electric surface plasmon polaritons mode.

FIGS. 17c-17d show an example of the magnetic surface plasmon polaritons mode.

FIG. 18 schematically shows the nanoantenna structure of the present invention.

FIG. 19a shows the comparative simulation of the electric field distribution in the individual split-ring resonator with a metal bar (cut wire) over it, placed in parallel to the gap.

FIG. 19b shows the resonance spectra from FIG. 19a in the THz frequency domain.

FIG. 19c shows the comparative simulation of the electric field distribution in the individual split-ring resonator with a metal bar (cut wire) over it, placed in a perpendicular direction to the gap.

FIG. 19d shows the resonance spectra from FIG. 19c in the THz frequency domain.

FIGS. 19e-19f show the magnetic field distribution in an individual split-ring resonator with a metal bar over it, placed in parallel and in perpendicular to the gap, respectively.

FIG. 20 shows the comparative THz spectrum for different number (from 0.5 to 3.0) of the split-ring resonators in a single metamolecule.

FIGS. 21a-21b show the simulation of the magnetic field distribution in the metamolecule composed of two split-ring resonators and a metal bar at 1.35 and 1.125 THz resonance frequencies, respectively.

FIG. 22 shows the formation of the EIT window in the THz spectrum of the sample due to excitation of the dark mode.

FIG. 23a-23c show the THz spectrograms of the nanoantenna structure of the present invention made of the metamolecules comprising two resonators and a metal bar.

FIG. 24 shows an optimised EIT window of the nanoantenna for two samples having different permittivity (epsilon), which is the function of a refractive index.

FIG. 25 shows the effect of the analyte concentration in the resonator sensing area on the EIT window.

FIG. 26 schematically shows the spiral nanoantenna array structure of the present invention.

FIGS. 27a-27b show the electric and magnetic field distribution, respectively, inside the spiral metamolecule without the container.

FIG. 27c shows the spectrum of the spiral antenna without the container.

FIG. 28a shows the dark mode of the electric field created at the 0.9 THz frequency in the contained spiral nanoantenna of the present invention.

FIG. 28b shows the S21 spectra of the contained spiral nanoantenna for different pixel pitches.

FIG. 28c shows the S21 spectra of the contained spiral nanoantenna for different pitches, when the pitch is changed in the direction of the electric field.

FIGS. 28d-28e summarise the SPP effects for various pitches for magnetic and electric fields, respectively, in different wave propagation directions.

FIG. 29a shows the S21 spectra of the contained spiral nanoantenna structure of the present invention for different pitches exhibiting spoof surface plasmon effect, when the spacing between the metamolecules is changed in the x-direction.

FIG. 29b schematically shows the contained spiral nanoantenna of the present invention and indicates the change in spacing between metamolecules in the x-direction.

FIG. 29c shows the spectrogram of the contained spiral nanoantenna structure of the present invention for different pitches exhibiting spoof surface plasmon effect, when the spacing between the metamolecules is changed in the x-direction.

FIG. 29d shows the S21 spectra of the contained spiral nanoantenna structure of the present invention for different pitches exhibiting spoof surface plasmon effect, when the spacing between the metamolecules is changed in the y-direction.

FIG. 29e schematically shows the contained spiral nanoantenna of the present invention and indicates the change in spacing between metamolecules in the y-direction.

FIG. 30a schematically shows the nanoantenna array structure of the present invention, wherein each metamolecule in the nanoarray is composed of a round-shape split-ring resonator having two splits in the ring and a metal bar under the round-shape split-ring resonator

FIG. 30b shows the electric field distribution at the first frequency mode in the metamolecule of the present invention comprising the split ring and the bar.

FIG. 30c shows the magnetic field distribution at the first frequency mode in the metamolecule of the present invention comprising the split ring and the bar.

FIG. 30d shows the spectra comparison of three nanoarray structures containing a round-shape split-ring resonator with two ring splits. These three nanoarray structures are different in having a wave container or bouncer. The first structure (green line) is a reference structure that does not have any wave container or bouncer. The second structure (violet line) has the metal bar as a wave bouncer under the split-ring resonator. The third structure (red line) has a metal square-shaped (box) as a wave container surrounding the split-ring resonator.

FIG. 30e shows the THz multi-resonance spectrum and fingerprint region of the metamolecule of the present invention comprising the split ring and the bar.

FIG. 30f demonstrates the difference in the FFT THz spectra between the square split-ring resonator structure described in the prior art and the nanoantenna structure of the present invention.

FIG. 31 shows an array of round (circular)-shape split-ring resonators having two splits in the ring and a metal square-shaped container (box).

FIG. 32a shows the nanoantenna array and a single metamolecule building this array. In this specific embodiment, each metamolecule is composed of a hexagon-shape split-ring resonator having at least one split in the ring and six metal hexagons surrounding said hexagon-shape split-ring resonator.

FIG. 32b shows the FFT THz spectra of the nanoantenna structure of FIG. 32a, where the array nests the complementary nanoarray structure into the outer plate.

FIG. 33a shows the FFT THz spectra of the nanoantenna structure of FIG. 32a for the sensor coated with the PMMA layers (pink spectra lines) vs. bare sensors without any PMMA layers (light blue spectra lines).

FIG. 33b compares the sensor response using the hexagonal fractal-design sensor of the embodiment shown in FIG. 32a with PMMA on the backside of the chip (green curve) versus the same sensor chip with PMMA on both sides of the chip (red curve).

FIG. 33c shows the rapid detection, tested on SARS-CoV-2 positive (red curve) and negative samples (green curve) using the hexagonal fractal-design sensor of the embodiment shown in FIG. 32a with the PMMA layers deposited on the surface of the sensor.

FIGS. 34a-34e show the testing chamber integrated into the housing and attached to the exhalation portion of the breathalyser of the present invention.

FIGS. 34f-34g show the exhalation portion of the breathalyser of the present invention.

FIGS. 34h and 34i show the testing chamber of the breathalyser of the present invention.

FIG. 35a shows the initial design of the breathalyser of the present invention, which is tubular and known as a Venturi tube.

FIGS. 35b and 35c show the velocity and pressure simulations, respectively, for the initial design using mass-flow inlet condition of 0.4 L/s of air.

FIGS. 35d and 35e show the similar Venturi tube design of the breathalyser of the present invention.

FIGS. 35f and 35g show initial simulation results for particles in the air guided around the chip.

FIGS. 36a and 36b show the design of the present invention with a focusing element.

FIGS. 36c and 36d show the velocity and pressure simulation, respectively, for the computational fluid dynamics model (CFD) of the design shown in FIGS. 36a and 36b.

FIGS. 36e and 36f show the results of the computer simulation for the design shown in FIGS. 36a and 36b.

FIGS. 37a-37c showing the simulation results for open centre designs of the breathalyser of the present invention, for different focus diameters.

FIG. 38a show the photos of the marketing candidate of the breathalyser of the present invention, and FIGS. 38b-38c show the design of this breathalyser.

FIG. 39a show the simulation model of the breathalyser shown in FIG. 38a, having 3.5 mm diameter focus.

FIG. 39b shows particle traces coloured by particles residence time hitting the chip surface of the breathalyser shown in FIG. 38a.

FIG. 40 demonstrates the Gaussian pulse, which is shaped as a Gaussian function.

FIG. 41 shows results of the simulation for velocity of the exhaled air flow inside the Venturi tube contouring at two normal midplanes of the testing chamber.

FIG. 42a shows the simulation of the Venturi design of the breathalyser of the present invention.

FIG. 42b shows the expanded view of the exhaust area of the breathalyser of the present invention having the Venturi design.

FIG. 43 shows the simulation results for the breathalyser of the present invention using the parameters of the SARS-CoV-2 virus.

FIG. 44 shows the photographs of the marketing candidate of the breathalyser system including the breathalyser of the present invention and a miniaturised THz spectrometer custom-made and manufactured by the applicant.

FIG. 45 shows the photographs of the marketing candidate of the lab THz spectrometer custom-made by the applicant for lab measurements of samples deposited on the microelectronic chip of the present invention, including swab samples.

FIGS. 46a-46b show the relationship between absorption peaks and CT values of viral load detected by transmission ratio method using nano absorber devices on quartz substrate.

FIG. 47 shows the feature classification based on wavelet analysis of the THz spectra comparing the +ve samples for SARS-CoV-2, Corona 1, 2, 3 and −ve samples.

FIG. 48 shows reproducibility in transmission ratio curves using CT22 samples indicating that measurements are repeatable over different days (27^{th} and 29 Apr. 2020).

FIG. 49 is a scatter plot showing the distribution of amplitude and frequency delta obtained using wavelet analysis. The −ve sample data groups in the range −0.24 to −0.21 THz, and also +ve samples appear to be distributed according to the viral load. An arbitrary line can be drawn which can separate +ve and −ve samples.

FIG. 50 is the wavelet analysis showing that as the number of layers (number of pipetting rounds) increases, the frequency delta reduces and moves closer to zero.

FIG. 51 shows the control sensor of the present invention (negative sample) (blue line) and the sensor of the present invention with the 1000-times diluted positive sample (red line).

### DETAILED DESCRIPTION

In the following description, various aspects of the present application will be described. For purposes of explanation, specific configurations and details are set forth in order to provide a thorough understanding of the present application. However, it will also be apparent to one skilled in the art that the present application may be practiced without the specific details presented herein. Furthermore, well-known features may be omitted or simplified in order not to obscure the present application.

The term “comprising”, used in the claims, is “open ended” and means the elements recited, or their equivalent in structure or function, plus any other element or elements which are not recited. It should not be interpreted as being restricted to the means listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a device comprising x and z” should not be limited to devices consisting only of components x and z. Also, the scope of the expression “a method comprising the steps x and z” should not be limited to methods consisting only of these steps. The term “consisting of” means “including and limited to”. The term “consisting essentially of” means that the device, method or structure components, steps and/or parts do not materially alter the basic and novel characteristics of the claimed device, method or structure.

Unless specifically stated, as used herein, the term “about” is understood as within a range of normal tolerance in the art, for example within two standard deviations of the mean. In one embodiment, the term “about” means within 10% of the reported numerical value of the number with which it is being used, preferably within 5% of the reported numerical value. For example, the term “about” can be immediately understood as within 10%, 9%, 8%, 7%, 6%, 5%, 4%, 3%, 2%, 1%, 0.5%, 0.1%, 0.05%, or 0.01% of the stated value. In other embodiments, the term “about” can mean a higher tolerance of variation depending on for instance the experimental technique used. Said variations of a specified value are understood by the skilled person and are within the context of the present invention. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges, for example from 1-3, from 2-4, and from 3-5, as well as 1, 2, 3, 4, 5, or 6, individually. This same principle applies to ranges reciting only one numerical value as a minimum or a maximum. Unless otherwise clear from context, all numerical values provided herein are modified by the term “about”. Other similar terms, such as “substantially”, “generally”, “up to” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein, the term “and/or” includes any and all combinations of one or more of the associated listed items. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity. As used herein, the singular form “a” “an” and “the” include plural references unless the context clearly dictates otherwise. For example, the term “a device” or “at least one device” may include a plurality of devices, including combinations thereof.

It will be understood that when an element is referred to as being “on”, “attached to”, “connected to”, “coupled with”, “contacting”, etc., another element, it can be directly on, attached to, connected to, coupled with or contacting the other element or intervening elements may also be present. In contrast, when an element is referred to as being, for example, “directly on”, “directly attached to”, “directly connected to”, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Sensing biological pathogens using THz nano-gap nanoantennae has been previously demonstrated by S. J. Park et al. in “Sensing viruses using terahertz nano-gap metamaterials”, Biomedical Optics Express 2017, 8(8), pp. 3551-3558 (https://doi.org/10.1364/BOE8.003551). They showed that THz nanoantenna is an effective sensing platform for detecting certain viruses, such as PRD1 and MS2, which are representative double-stranded DNA and single-strand RNA viruses, respectively. An exemplary prior-art nanoantenna structures arranged in a periodic array and configured to detect and transmit signals from the sample in a terahertz (THz) frequency range, and its fabrication, are described in Ji-Hun Kang et al., “Terahertz wave interaction with metallic nanostructure”, Nanophotonics 2018, 7(5), pp. 763-793. It should be noted, however, that the so-called “terahertz nano-gap metamaterials”, the term used nowadays by many including S. J. Park et al. (2017) and Ji-Hun Kang et al. (2018), has nothing to do with actual metamaterials, and this will be explained below.

The present invention describes embodiments of a microelectronic sensor for non-invasive and label-free chemical detection and biomolecular diagnostics of analytes in a raw sample, comprising a microelectronic chip and a sample collection system attached to said microelectronic chip or incorporating said microelectronic chip, said sample collection system is suitable for sample collection of a raw sample taken directly from a subject being tested without any purification and without any chemical or biological separation, and for delivery of the sample to said microelectronic chip, wherein said microelectronic chip comprises
(a) a nanoantenna structure, said nanoantenna structure is arranged in a periodic array of metamolecules and configured to detect and transmit signals through said sample in a terahertz (THz) frequency range; and
(b) an integrated circuit for storing and processing signals in a THz frequency domain, and for modulating and demodulating radio-frequency (RF) signals;
characterised in that each of said metamolecules in the array is composed of at least one split-ring resonator and a wave container or a wave bouncer, said wave container confines and said wave bouncer bounces electromagnetic waves received from said at least one split-ring resonator, both the wave container and the wave bouncer are designed to excite a dark mode in said at least one split-ring resonator, followed by coupling the excited dark mode back into said at least one split-ring resonator.

In some embodiments, the split-ring resonators are selected from rod-split-rings, square-split rings, nested split-rings, single split rings, deformed split-rings, spiral split-rings and spiral resonators. In other embodiments, the wave container is selected from a metal bar, metal ring or metal square. In a specific embodiment, each metamolecule is composed of two square split-ring resonators and a single metal bar over the resonators, said metal bar is capable of exciting a dark mode in said resonators, followed by coupling it into said resonators. In other specific embodiments, each metamolecule is composed of a spiral resonator and a metal ring container surrounding and confining said spiral resonator, said metal ring is capable of exciting a dark mode in the spiral resonators, followed by coupling it into the said spiral resonator. In a particular embodiment, each metamolecule is composed of a split-ring resonator and a metal bar over said split-ring resonator, said metal bar is capable of exciting a dark mode in the split-ring resonator, followed by coupling it into said split-ring resonator.

In one aspect of the present invention, the nanoantenna structure of the present invention comprises an actual metamaterial deposited on top of the substrate and arranged in a periodic array. Such metamaterial nanoantenna is designed to detect and transmit electromagnetic signals from the sample in a terahertz (THz) frequency range. In general, “metamaterial” is an arrangement of artificial structural elements, which are defined herein as “metamolecules” or “individual unit cells”, designed to achieve advantageous and unusual properties unattainable in natural media. A metamaterial gains its properties from its unique structure rather than composition. Metamaterials are generally engineered periodic or non-periodic material composites with subwavelength structures. They are employed to sense molecular vibrational fingerprints in the mid to long infrared wavelengths. Their physical properties, such as the dielectric constant, permeability, and conductivity, can be arbitrarily designed by changing the structure and size of the periodic lattice. Metamaterial and molecular resonance coupling are a result of near field interaction. Therefore, many interesting phenomena can be realised by tailoring the geometry of the metamolecules, with immense application potential such as meta-lenses and related wavefront regulation in metasurface, negative index media materials, polarisers and metamaterial absorbers.

Examples of metamaterials are aerogel, graphene, carbon nanotubes, mother-of-pearl receiving its rainbow colour from metamaterials of biological origin, bubble wrap, which is a favourite packing-based stress reliever that can absorb masses of energy except made from aluminium, titanium foam clinging to the shape of a simple foam and perfect for replacing human bones, molecular superglue, amorphous metals with a disordered atomic structure made by quickly cooling molten metals, fibroin-based artificial spider silk, D3o polyurethane-based energy-absorbing material, bioluminescent bacteria-based glowing materials, graphene and graphene aerogel.

The term “metamaterial” has been significantly broadened nowadays and refers to many different types of artificial materials which can customise the electrical and magnetic properties of a material to perform certain applications. Within this broad definition are electromagnetic metamaterials, electrical resonance metamaterials, magnetic resonance metamaterials, bright-mode metamaterials, dark-mode metamaterials, symmetric metamaterials, asymmetric metamaterials, surface plasmon polariton metamaterials, fano-coupled metamaterials, trapped-mode metamaterials, bright-and-dark-mode coupled metamaterials and electromagnetic induced transparency metamaterials.

In a particular embodiment, the nanoantenna of the present invention has a square split-ring resonator design, similar to that of S. J. Park et al. (2017) and Ji-Hun Kang et al. (2018). Near-field electromagnetic coupling responses indicate that the resonance is caused by coupling of the electric inductive-capacitive (LC) elements between the split-ring resonator units in the nanoantenna array. However, the resonant frequencies of this design cannot be altered. The LC split-ring resonators can only filter or absorb certain electromagnetic spectra passively. In other words, the nanoantenna design described in S. J. Park et al. (2017) and Ji-Hun Kang et al. (2018) cannot produce any time-domain resonance frequency shift. Being purely electric in their nature, these split-ring resonator units are capable of measuring only capacitance and indicating only signal amplitude changes. This drawback of the nanoantenna structure described in Park et al. (2017) and in other prior art publications, such as Ji-Hun Kang et al. (2018), other downsides and possible solutions will be discussed in detail below.

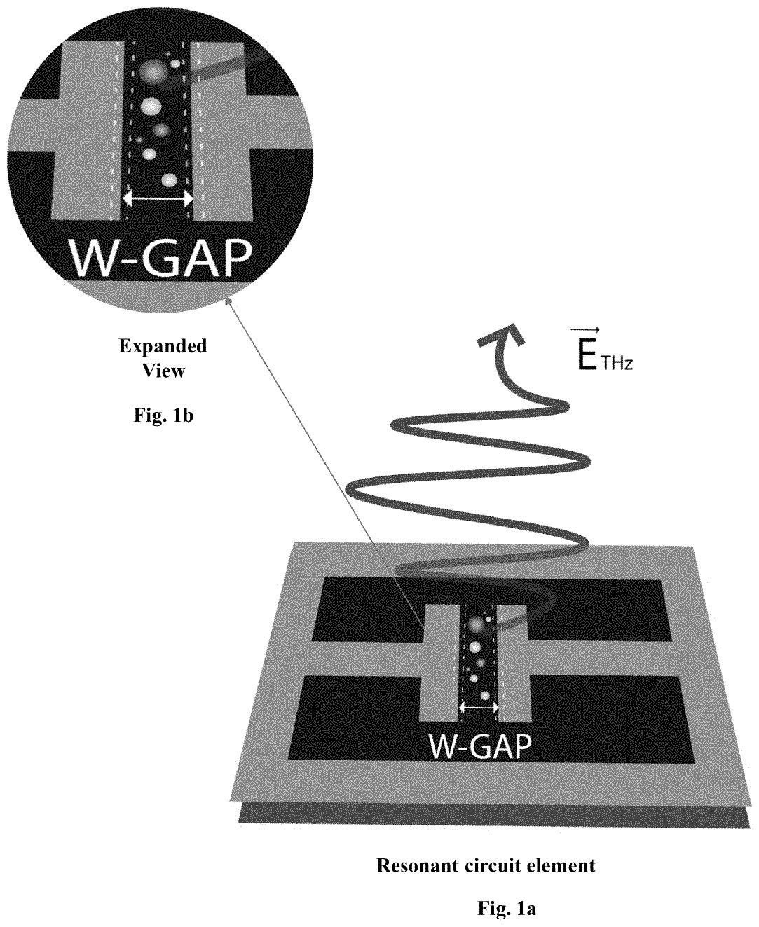

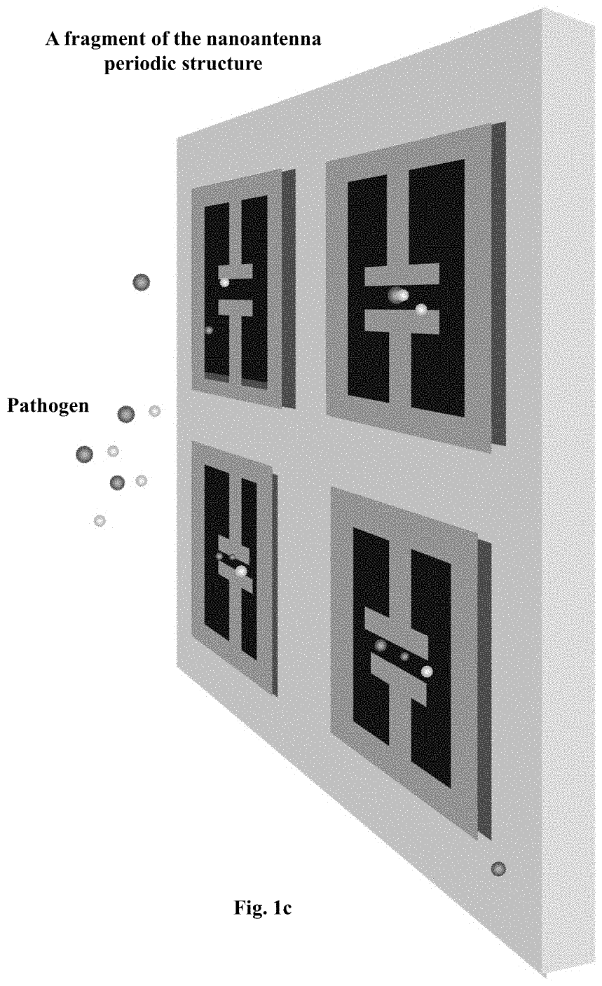

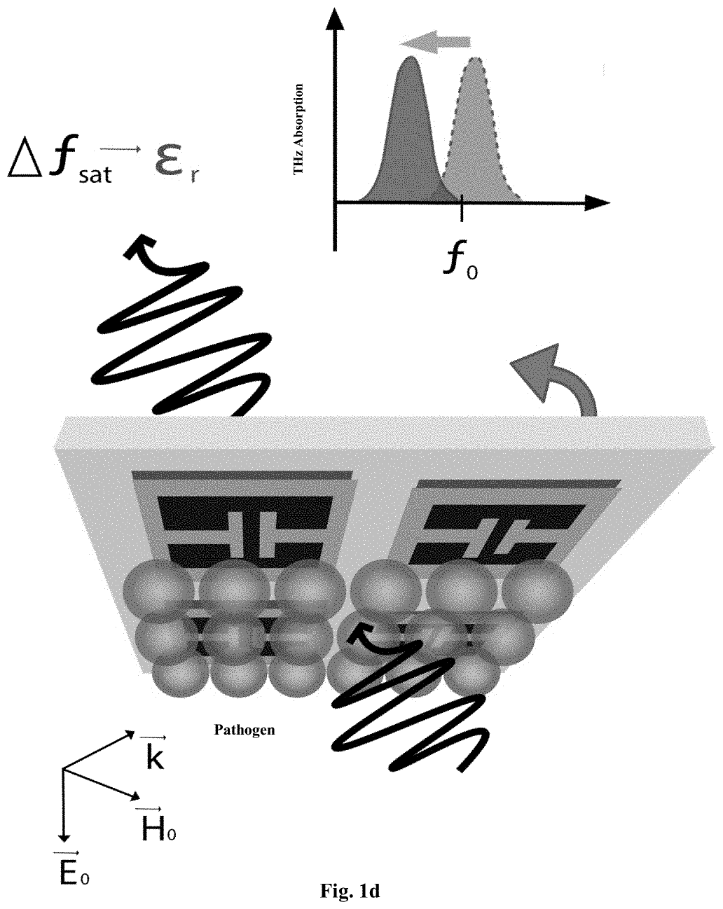

The resonant frequency of the split-ring resonators is strongly dependent on the presence of dielectric in capacitive nano-gaps in the structure, and a shift of the resonant frequency occurs when the dielectric constant changes. Therefore, it is possible to detect nano-size analyte molecules or pathogen particles being tested with high sensitivity and to count their number located in the nano-gap area using a tightly defined detection volume of the split-ring resonators. FIGS. 1a-1b show a schematics of THz nano-gap split-ring resonators used for sensing pathogens, such as viruses. FIG. 1c schematically shows the nanoantenna structure, deposited on the substrate, arranged in a periodic array and configured to detect and transmit signals through said sample in a terahertz (THz) frequency range. FIG. 1d schematically shows the detection process of a viral pathogen with the nanoantenna structure of the present invention.

The parameter measured is the amplitude change in the THz spectrum transmitted through the nano-gap split-ring resonators after deposition of the sample. The resonant frequency of the split-ring resonators is primarily determined by geometrical factors such as the nano-gap width and resonator dimensions, for example the sidearm length in the above figure, and also by the substrate refractive index. Two major parameters considered then are capacitance of the nano-gap and inductance of the sidearm, as with any traditional LC circuits. To account for the dielectric environment of the metamaterial, an effective refractive index, which is a linear combination of the refractive indices of substrate and air, is introduced and the resonant frequency varies inversely with this refractive index. Additional dielectric materials such as analytes being tested in the sample produce a change in the effective dielectric constant in the split-ring resonator nano-gap areas, and a shift occurs in the THz transmission function, which is recorded as a signal specific for each and every particular analyte tested.

In general, “electromagnetic metamaterials” is a term for all metamaterials that utilise electric and magnetic response or resonance in a metamolecule or in any array of metamolecules to produce a metasurface. This definition is more limited to bright-mode and dark-mode metamaterials. Such structures respond electrically and magnetically to incident electromagnetic fields, resulting in electric, magnetic, or magneto-electric resonance with respect to the direction of the THz wave propagation. For normal incidence THz radiation, the electric and magnetic fields lie in the plane of the metamaterial surface. Therefore, the electromagnetic metamaterials only interact with the electric field of the THz excitation radiation, which is the case of all metamaterials, unless special and unique techniques are utilised together with novel geometry and other multi-mode theory models. For oblique angle incidence and prism coupling, both electric and magnetic fields can be excited and can produce both electric and magnetic resonances. However, the THz free-space coupling in these types of setups is weak and leads to a very low interaction strength between the electromagnetic radiation and the sample. For more advanced approaches, a waveguide is used to couple the THz radiation directly into a metamaterial.

“Electromagnetic metasurfaces” is a term for various artificial sheet materials with sub-wavelength thickness that allow or inhibit the propagation of electromagnetic waves in desired directions. For example, metasurfaces have been demonstrated to produce unusual scattering properties of incident plane waves or to guide and modulate surface waves to obtain desired radiation properties. Electromagnetic metasurfaces can be either structured or unstructured with sub-wavelength-scaled patterns in the horizontal dimensions. The most important difference between metamaterials and metasurfaces is that the metasurfaces modulate the behaviours of electromagnetic waves through specific boundary conditions, rather than constitutive parameters in three-dimensional space, which is commonly exploited in metamaterials. Metasurfaces may also refer to the thin two-dimensional layered counterparts of metamaterials. They can be designed such that they have a resonance at a molecular vibrational frequency. Mode splitting results from the coupling of two electromagnetic field distributions, or modes, spatially and/or temporally.

Analogous to the atomic system, in symmetric structures, super radiant or “bright modes” exhibits a broad resonance or short lifetime that couples strongly with incident far field radiation, producing broad and lossy resonance. They mostly radiate into free space and therefore have heavy losses. With the introduction of asymmetry in the metamolecule geometry, trapped or “dark modes” can be excited. These dark modes provide a resonance or a long lifetime that weakly couples with an excitation far field (i.e. to the free space) and therefore, provide high values of the quality factor Q.

The split-ring resonator LC circuits described, for example, in Park et al. (2017), act as metamolecules forming a metasurface. Dark modes in these “pseudo-metamolecules”, which are essentially the nano-gap LC circuits are mostly affected by their “arm” length. Width and gap variations of the LC circuits only slightly change the resonance frequency. The major role played by the arm length, in comparison to the other parameters, clearly originates from the fact that the longitudinal oscillations of charges are responsible for the observed “dark-mode” plasmon resonance. Thus, Q-factor of the dark-mode created by symmetry breaking (using asymmetric metamolecules) is much higher and much more sensitive than of the bright mode. A detailed comparison of the bright and dark modes is provided by N. E. J. Omaghali et al. in “Optical Sensing Using Dark Mode Excitation in an Asymmetric Dimer Metamaterial”, Sensors 2014, 14, pp. 272-282. They concluded that for both the bright and dark mode, Q-values take into account the relative sharpness of the resonance peaks only. For the bright mode, the resonance peak width broadens at a slower rate compared to the dark mode, as a consequence of its higher coupling to the free space, which results in an almost independent behaviour of its Q factor with length asymmetry. Therefore, the asymmetry seems to be the dominant factor responsible for the excitation of the dark modes.

“Electrical resonance metamaterials” is a subclass of the electromagnetic metamaterials which are symmetrical split-ring resonators and which are (at normal incident) electrically resonant and exhibit a strong resonant permittivity at the “same frequency” as the magnetic resonance. In these metamaterials, the high symmetry eliminates any possible magneto-optical coupling effect due to bianisotropy that implies double polarisation mechanism and anisotropic response. Bianisotropic materials acquire magnetic (electric) polarisation when excited by electric (magnetic) external field.

Symmetric metamaterials, especially split-ring resonators only operating in the LC mode, such as those described in Park et al. (2017), can be designed based on group theoretical methods. As mentioned above, the fundamental mechanism eliminates any magneto-optical coupling effects relating to bianisotropy and yields electrically resonant structures only. Furthermore, in all symmetric metamolecules, the magnetic response is suppressed. Thus, such LC resonators function as localised particles from which one can construct a purely electrical resonant response, i.e. bright mode as defined above. Nanoantenna structures based on such electric resonators only respond to the dielectric constant (permittivity) changes of the material located in the electric field and cannot sense changes in a refractive index. This applies to any LC circuit in which the ratio of the capacitance of the capacitor using some dielectric in the capacitor is measured in comparison to a capacitor in vacuum. For this reason, electrical resonance metamaterials require a gap (or split) in their split-ring structure to be filled with the sample, as it would be done with a dielectric material in a capacitance measurement.

### Spectral Analysis Using a Wavelet Transform

A spectral analysis using the wavelet transform was used to initially design the microelectronic sensor of the present invention. A spectral signal is composed of a series of resonant peaks with a characteristic width and amplitude, each cantered at a particular frequency. For most applications, such as the COVID-19 diagnostic test, measuring the properties of certain peaks accurately is essential for correct interpretation of the spectra. The wavelet transform technique aims to maximise the accuracy of the peak analysis by decomposing the original spectrum into its constituent resonances, followed by removing the interference induced by overlapping peaks and/or baselines.

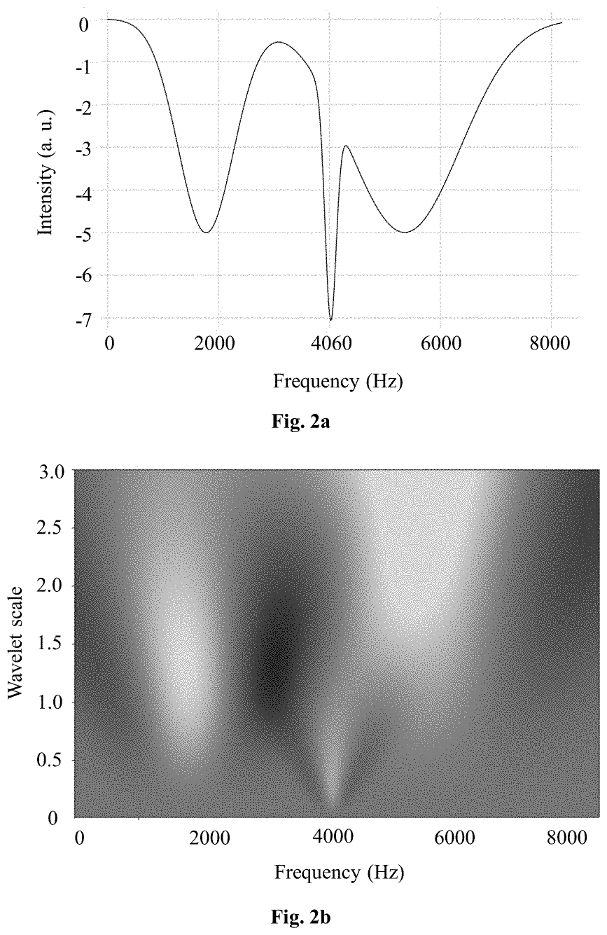

A wavelet transform works by mathematically convolving a 1D-signal with a series of orthonormal wavelets in order to produce a decomposed 2D signal in the wavelet domain (w-space). The w-space is a wavelet function which is dependent on a wavelet width-scale (a), wavelet shift (b) and original spectrum (f). The w-space can therefore be thought of as a 2D surface that represents the relative amplitude of a peak of width a located at position b. Reference is now made to FIG. 2a showing an example of a simple spectrum, while FIG. 2b shows the corresponding wavelet transform. The peaks in FIG. 2a correspond to light bands in FIG. 2b. Thus, FIG. 2a demonstrates an exemplary generated spectral signal and FIG. 2b shows the corresponding Laplacian wavelet representation. The wavelet scale on the y-axis represents the width-scale parameter (a), and the x-axis represents the wavelet shift (b). A horizontal slice through the wavelet transform represents convolution of the original signal with a single wavelet of a fixed width. As an example, the bright yellow region at around 1800 Hz tells us that in this frequency range, there is a peak with width-scale 1.0-1.5.

One of the most common requirements for spectral analysis is measuring the frequency position and amplitude of a particular peak accurately. When multiple peaks are overlapping with each other, or when there is an underlying baseline in the signal, the measured peak characteristics can contain large errors if measured naively. For example, if one tries to measure a resonant frequency by finding the peak maximum, and if there is a non-constant baseline, the maxima position will not correspond to the central resonance frequency (this is similar to shift of the maximum of a parabola with increasing the gradient of some linear baseline). A similar problem occurs when the peak being measured overlaps with the tail of a neighbouring peak, since the interfering signal affects the derivative of the summed signal, and therefore the position of the maximum.

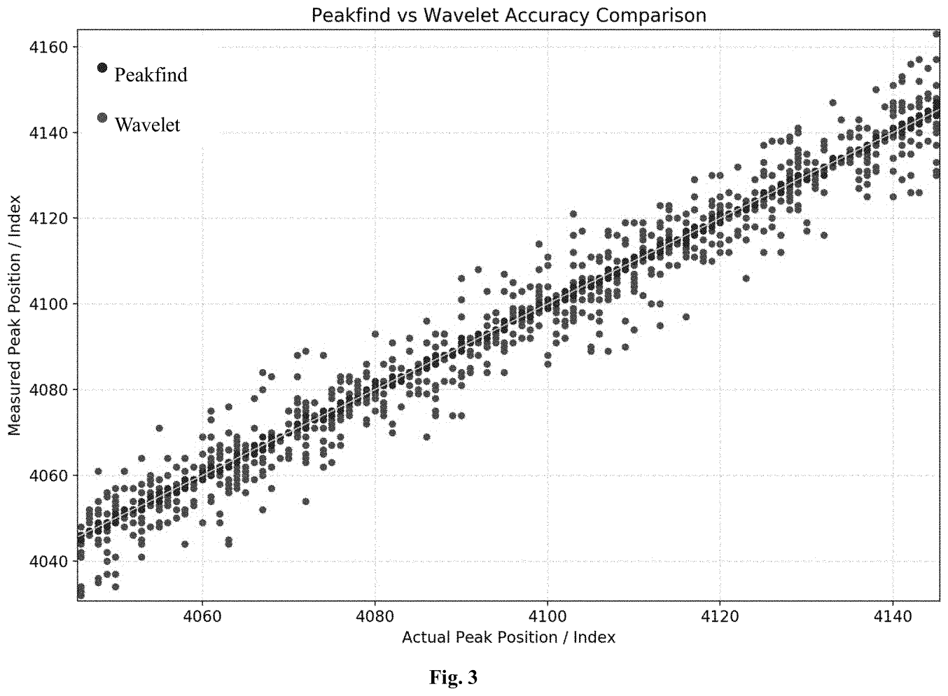

In order to prove the effectiveness of the wavelet technique, a simulation was created to demonstrate its superior accuracy when compared to more conventional peak-finding algorithms. Thousands of artificially generated spectra were randomly generated in Python, and the ‘real’ resonant frequencies for each peak were recorded. Then, a peak of interest was selected from each spectrum and its resonant frequency was measured separately using both the peak-find method and the wavelet method (taking a horizontal slice at the correct scale through the transformed dataset and finding the maximum value within that slice). A measured resonance frequency that was close to the generating resonance frequency recorded earlier indicates a high measurement accuracy. FIG. 3 clearly shows that the wavelet technique is far more accurate at determining resonant frequencies when compared to peak-find (maxima-based approaches on spectral data).

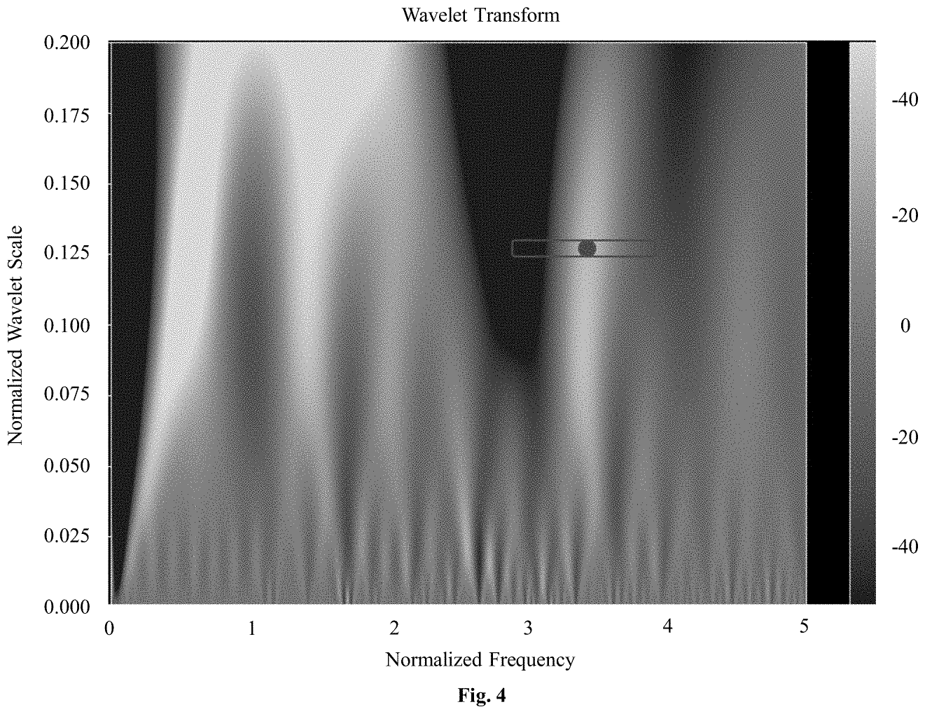

The metric of interest for characterising SARS-CoV-2 samples from generated spectral data is a function of the frequency shift and amplitude change of the sensor's resonance frequency when compared to a reference measurement (there may be multiple such resonances in future sensor designs). Since the resonance peak is quite broad, and many superposed peaks are interfering with the profile, the wavelet transform is ideally suited to isolate the sensor antenna resonance and measure its spectral characteristics. For each reference-sample pair, two transforms are generated and a horizontal region of interest is selected. The same process is then repeated for all other measurement pairs in the dataset, but the original region of interest is kept constant. For a reference-sample pair, the maximum value within the region of interest is found and the frequency-shift and amplitude shifts are determined. FIG. 4 shows an example of a reference transform, marked with a red region of interest and the associated maximum value.

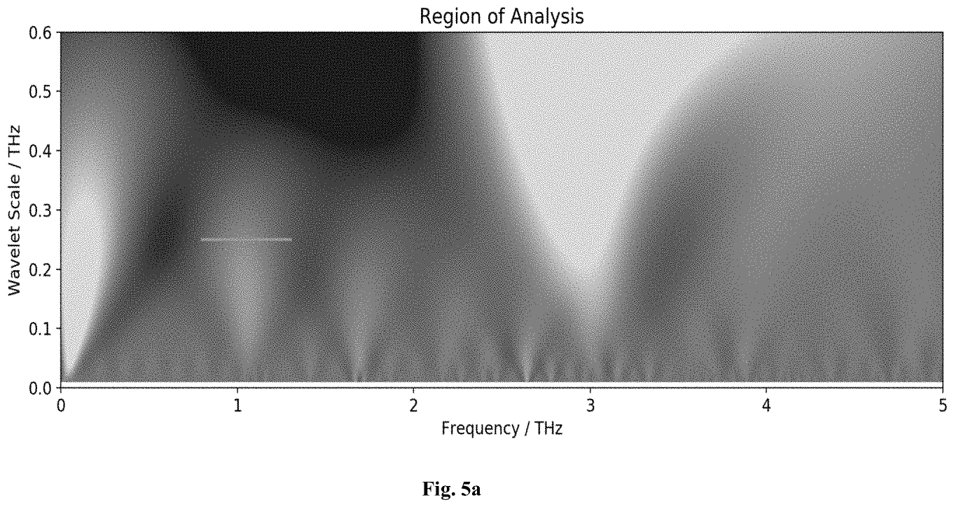

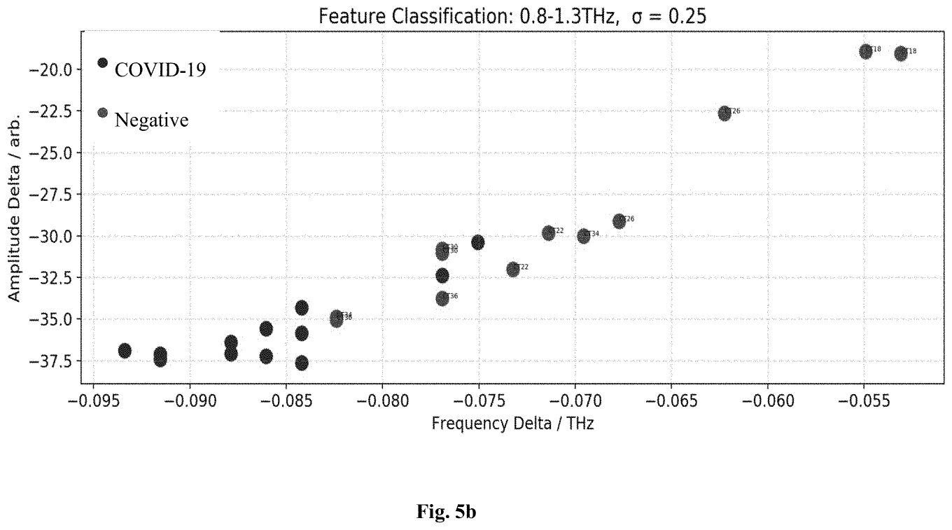

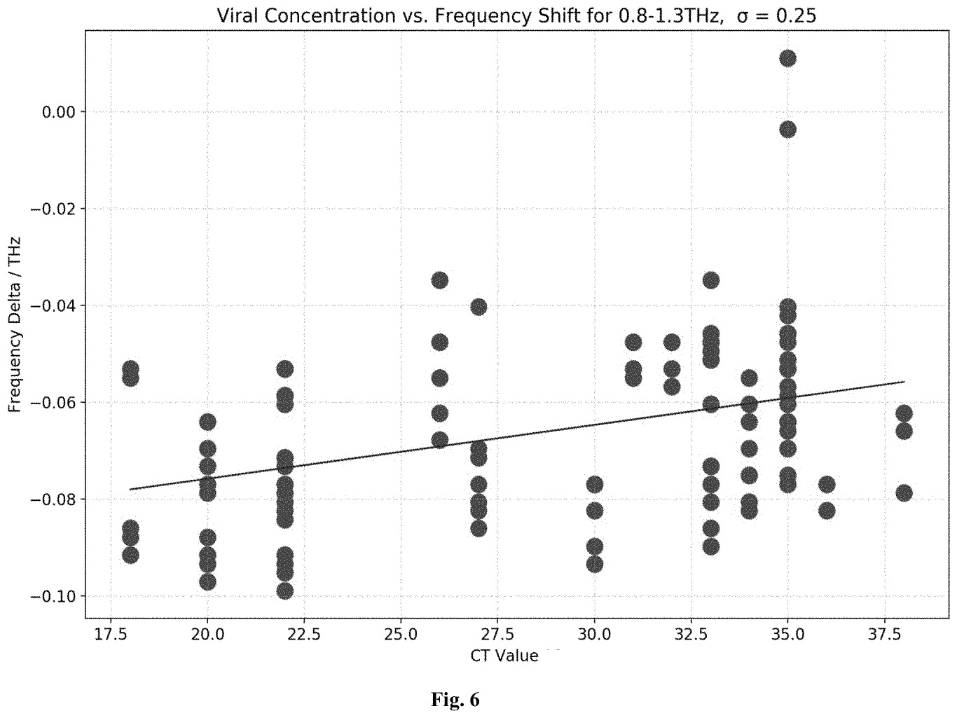

When the same calculation is applied to all reference-sample pairs, the frequency and amplitude shifts can be plotted for each on a scatter plot. Ideally, the positive samples will cluster in one region of the plot, and be separate from the negative cluster. FIGS. 5a-5b shows the results from a “good” dataset, although the sample count is quite limited. FIG. 5b represents one slice of the wavelet shown in FIG. 5a. Once sufficient number of samples are collected, the decision region can be calculated (e.g. by maximum likelihood estimation) and the model can be tested using blind samples. The user can interactively change the position of the analysis region using the mouse, and the scatter plot will then update in real time. In addition, it is possible to plot the relationship between the cycle threshold (CT) value of the sample (analysed with the PCR technique) and the shifts, as shown in FIG. 6, for example.

Thus, as demonstrated above, the wavelet transforms enable very high-accuracy resonance measurements, where multiple peaks of interest can be isolated and measured reliably without interference. This technique can also be used in isolation or as a first step in a more complex algorithm. Its implementation is flexible and allows the user to select a region of interest visually. In general, wavelet transform is baseline independent, allows extracting and separating overlapping peaks, scale-independent, detects water signature changes and provides visual representation of spectral components. It is highly sensitive to peak shifts and amplitude changes, does not rely on subjective parameters, easy to slice through data and reversible.

### Clinical Experiments Using THz SARS-CoV-2 Rapid Detection Platform

The clinical experiments were conducted to classify the COVID-19 positive (+ve) and negative (−ve) samples with high accuracy using the microelectronic sensor and contactless and reagent-free sensing technology of the present invention, followed by a validation study using randomised samples with unknown classification. The sensor of the invention containing a THz nano-antenna optimised for detecting SARS-CoV-2 virus was designed using numerical FEM simulations (COMSOL) as a guide for fabrication.

Clinical samples (+ve and −ve) were generously provided by the Institut für Virologie, Universitatsklinkum des Saarlandes where the experiments were performed beginning Mar. 30, 2020 (supervised by Prof Dr. med. Sigrun Smola). The initial data from these studies were applied to refine three analytical approaches used to classify the clinical swab samples. The results in this summary study pertain to the latest experiments performed on Apr. 14, 2020 where three +ve and three −ve samples were provided as control along with five unlabelled/randomised samples for blind testing and prediction.

The following sensors (chips) have been tested:

Control group: (+ve) SB_20, SB_21, SB_22;
(−ve) SB_23, SB_24, SB_26;
   Blind test: SB_27 (+ve), SB_28 (−ve), SB_29 (−ve), SB_30 (+ve), SB_31 (−ve).
   Three analytical approaches have been developed for analysing the obtained data.

### Approach 1

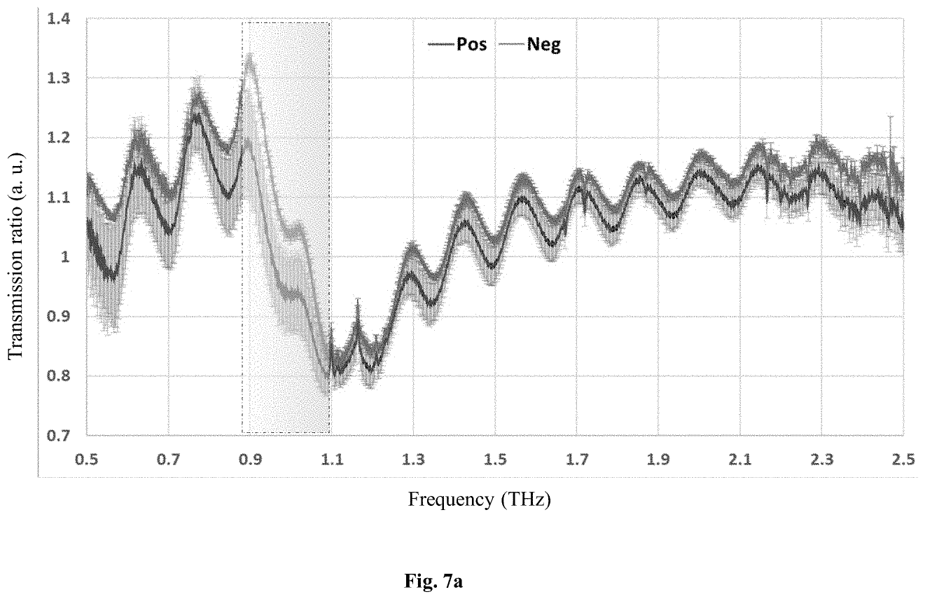

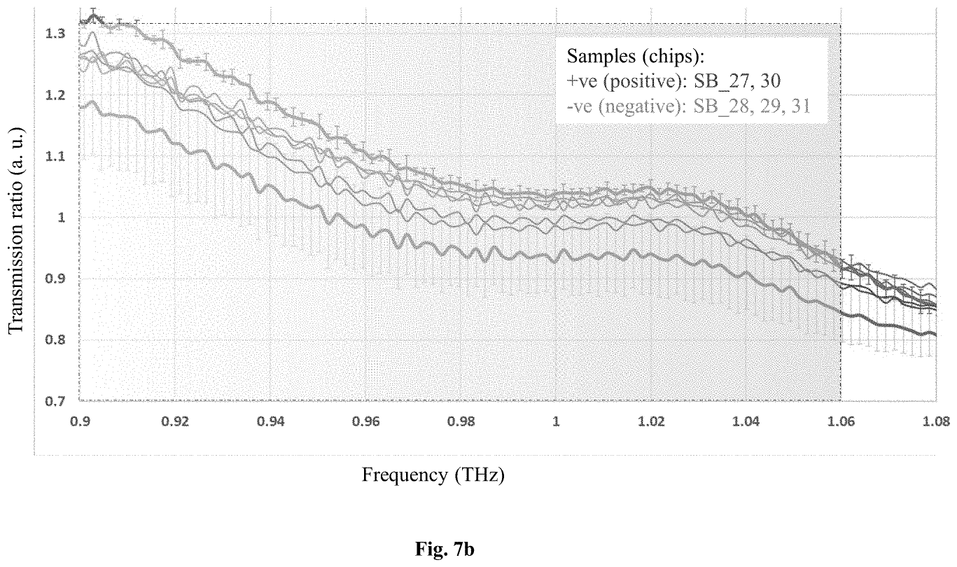

Reference is now made to FIG. 7a showing the relative transmission spectra (the relative intensity) of the virus signal in a frequency range 0.9-1.1 THz (shaded area). The relative transmission spectra have been obtained by scaling the raw transmission data of the samples by their corresponding reference measurement (without sample). These data are shown in the frequency range 0.9 THz and 1.06 THz, which is the frequency range that corresponds to the virus frequency, and where the control data were distinguishable (red vs green). FIG. 7b shows the spectra in the frequency range 0.90-1.08 THz, from blind tests that fall within the standard deviation of control. The data plotted are the averages for each group (+ve and −ve) along with their standard deviations, shown as whiskers to indicate the non-overlapping regions. The blind test data shown in FIG. 7b, when plotted in the same manner, fall in its respective category, thereby allowing easy classification.

### Approach 2

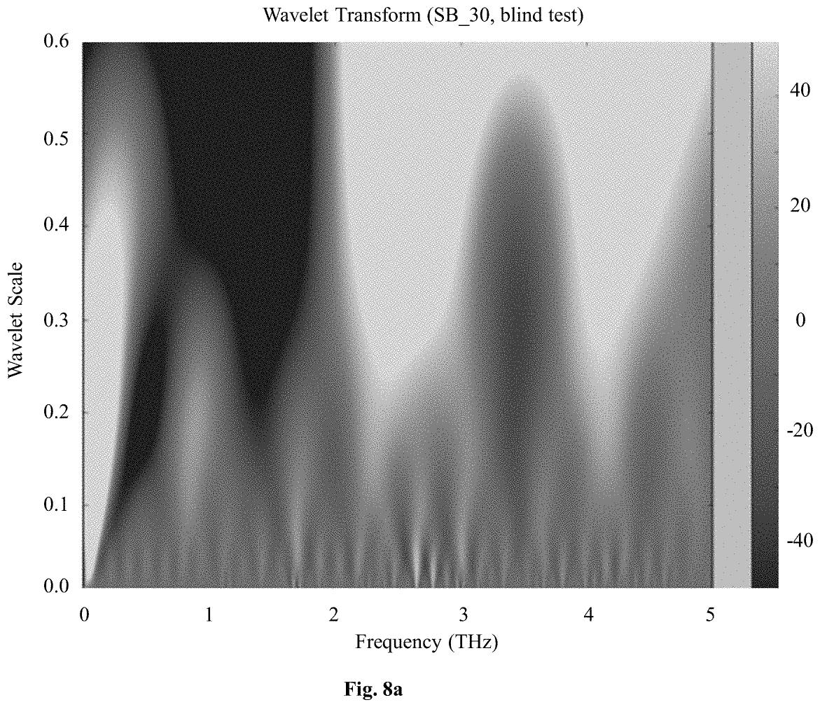

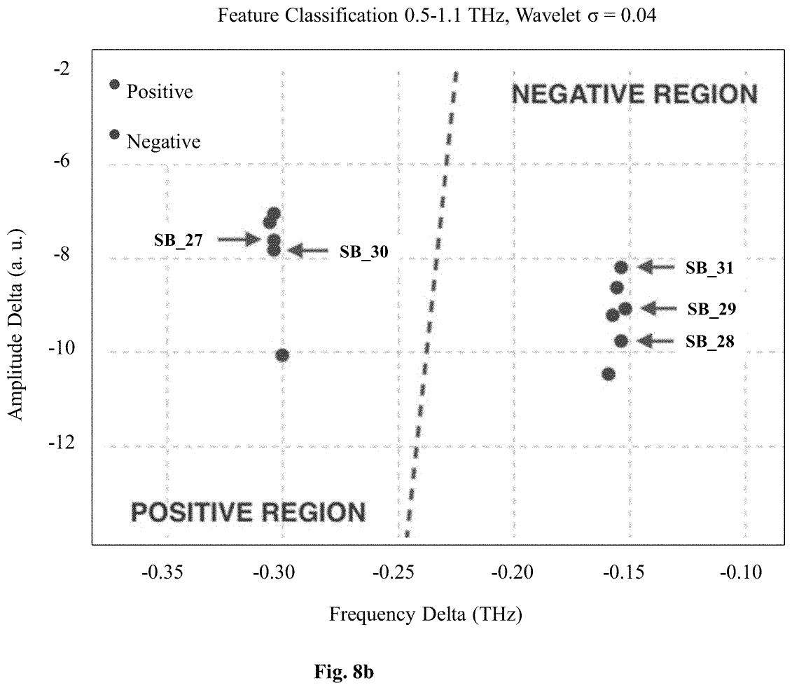

As described above, a novel spectrum analysis technique using wavelet transforms have been developed to classify samples, by which the raw frequency data are decomposed by peak location, width, and amplitude, thereby allowing the visual extraction of a signal of interest. This is followed by running an algorithm that searches the transformed space in order to optimise the classification capability of the final prediction. An exemplary wavelet transform for SB_30 positive (+ve) blind test is shown in FIG. 8a, while FIG. 8b shows the absolute classification of positive and negative samples, including the prediction for unknown-blind and randomised samples.

### Approach 3

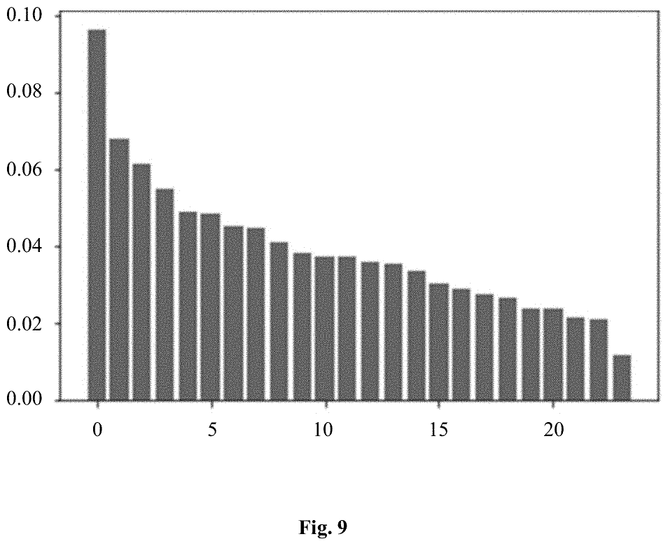

This is a machine learning approach, which consists of two models: the random forest classifier and the AdaBoost classifier. The models were trained on a large spectra dataset, each split and labelled into 25 sections of equal width. Each model was then evaluated on a test dataset, from which additional plots that identified the most critical features of a spectrum required for COVID-19 samples classification were generated. In this regard, FIG. 9 shows feature importance extracted using the AdaBoost algorithm. The predicted outcome by PCA classification is shown in Table 1 below. As clearly seen from the table, all three approaches consistently predicted the same outcome.

TABLE 1
Predictions for clinical blind test based on the three approaches.

| Chip ID | Applicant's Prediction | Correct Classification (verified by) |
|---|---|---|
| SB_27 | +ve | Dr. Stefan Lohse |
| SB_28 | −ve | Dr. Stefan Lohse |
| SB_29 | −ve | Dr. Stefan Lohse |
| SB_30 | +ve | Dr. Stefan Lohse |
| SB_31 | −ve | Dr. Stefan Lohse |

The following conclusions from these clinical experiments and machine learning tests can be made by the frequency shift comparison:
1) Automated peak detection can be implemented;
2) Peak position can be used as a variable for further classification algorithms to take the decision; and
3) Classification can be performed, for example by supervised learning.

### Split-Ring Resonators Nanoantenna Operating in a Pure LC Mode

### Sample Preparation

Nasal swabs were collected from a risk group of fourteen patients, seven of whom tested positive and seven that tested negative for COVID-19. The swab samples were collected diluted in a buffer solution using standard procedures. All samples were subjected to PCR amplification to test for SARS CoV-2 virus. Based on results from PCR testing, samples were categorised into COVID positive and COVID negative groups.

For feasibility study, five sensor chips of the present invention containing split-ring resonators nanoantenna operating in a pure LC mode were drop cast with 7 μl liquid sample loads pipetted from COVID-19 negative sample sets, followed by drying. The drying was assisted by ventilation in fume hood and took almost 20 mins. This step time is further reduced by an order of magnitude using the applicant's proprietary microfluidic system for microfluidic-assisted evaporation under development, which will be disclosed in a separate patent application. After drying each loaded sensor chip was measured using a THz spectrometer operated in a transmission mode. After each measurement, the loaded sensors were flushed with PBS solution and dried again before retaking the reference spectrum. This was done to check whether the sensor load was cleared by comparing the measurement to the previously acquired reference spectrum. Reproducible reference spectra before loading and after cleaning were used to determine whether the sensor was acceptable for subsequent tests. The same procedure was followed for the COVID-19 positive sample.

### Measurements

Menlo Systems® THz spectrometer was used in this study. The instrument's control software allows a variety of acquisition options, two of the most important ones being the duration of each acquisition in the TDS and the number of averages taken on the collected FFT spectra in every experiment. By increasing the number of averages, the noise contribution was minimised. It was found in the present experiments that 300-500 averages give practically identical results. Therefore, most of the measurements were performed using 400 averages. The duration of the measurement in the TDS is 400 ps. This allows computation of high resolution FFT in the spectral range form 0-3 THz, which contains useful information about the samples under study. Further increase in the acquisition time negatively impacts the SNR (signal-to-noise ratio) beyond 3 THz. Keeping this in mind, this region of the spectrum was not used for data analysis. Experiments were performed over five days going over each patient group one by one by reusing the five available sensors.

### Reference Calibration

The sensor chips of the invention containing split-ring resonators nanoantenna operating in a pure LC mode was designed to resonate in a band of 1.2-1.3 THz where they absorb THz radiation. This manifests as a dip in the transmission spectra obtained from the measurements. Design variations in the structural geometries of the sensor can provide broadening and shifting of this absorption band. Therefore, to observe relative variations in absorption due to the presence of various biological materials on the nanoantenna surface, a reference measurement is taken using a sensor without any sample load and after cleaning prior to measurement with the clinical specimens.

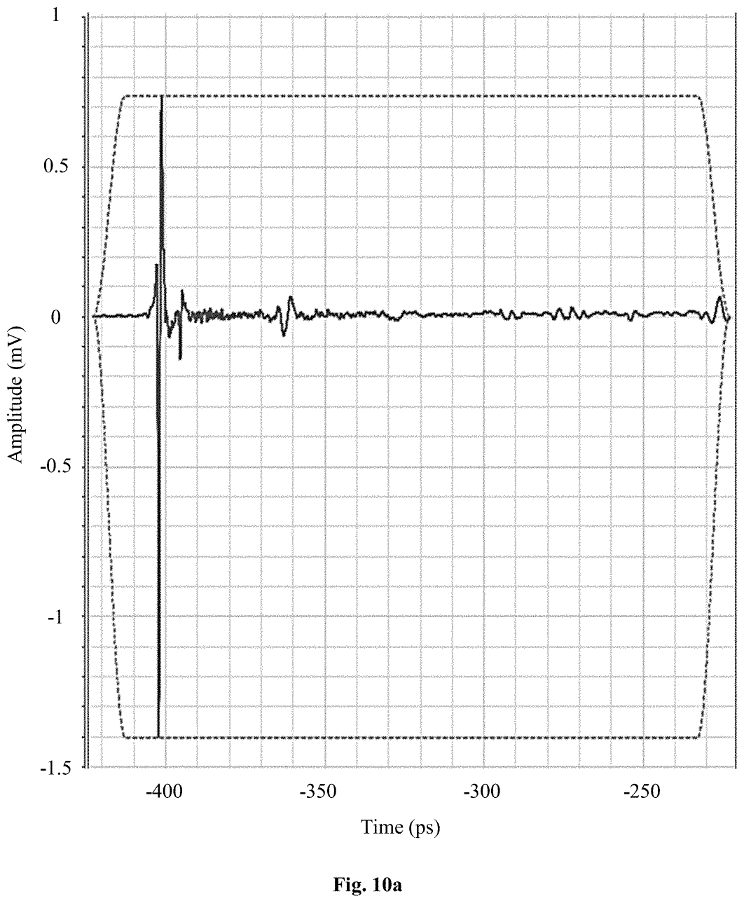

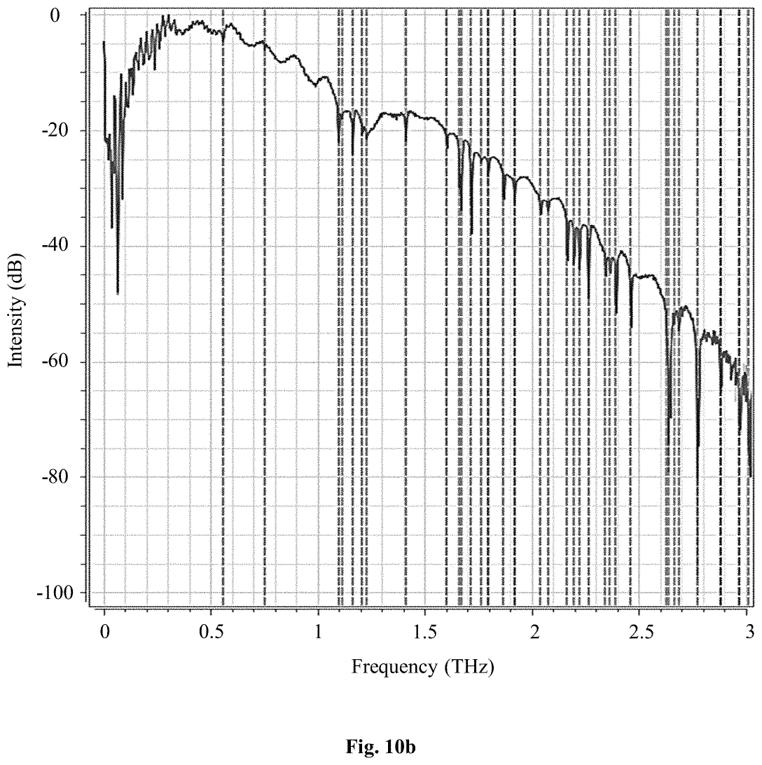

FIG. 10a shows a spectrum taken with a TDS-THz spectrum analysis software, while FIG. 10b shows the corresponding FFT (Fast Fourier Transform) of this reference measurement. A clear dip is observed in the transmission FFT data at 1.23 THz, which corresponds to the resonance energy of the nanoantenna used in this measurement. The vertical dashed lines in the FFT mark known absorption lines of water. These appear as spike shaped dips in the transmission spectra and align well with the known water lines indicating the presence of water on the sensor chip nanoantenna surface, most likely arising from the PBS residues after drying.

### Proof of Concept

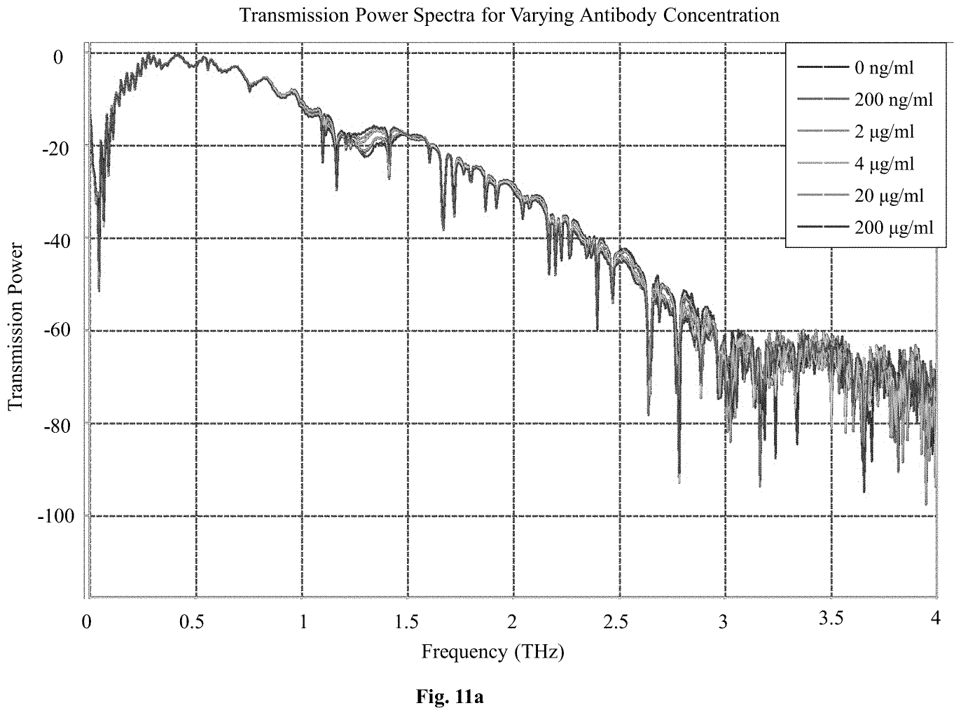

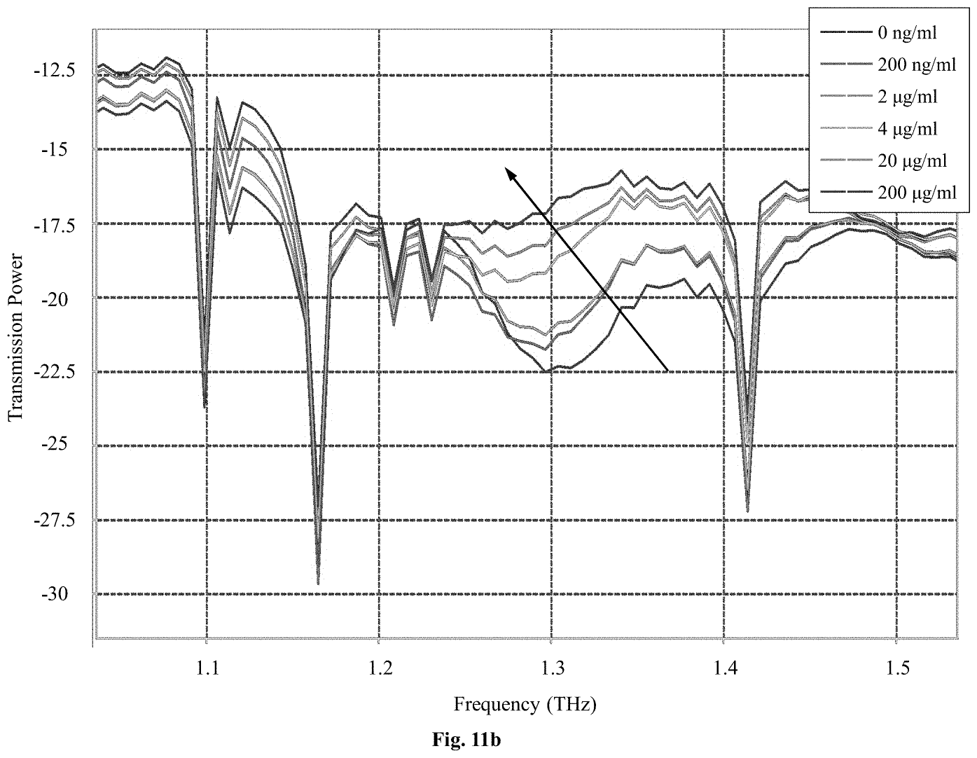

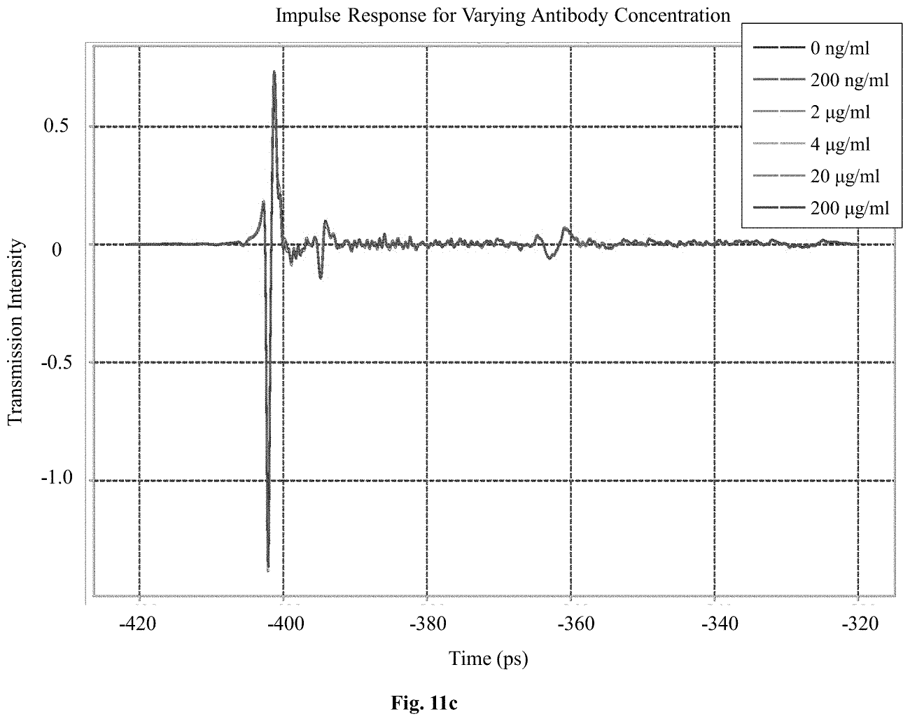

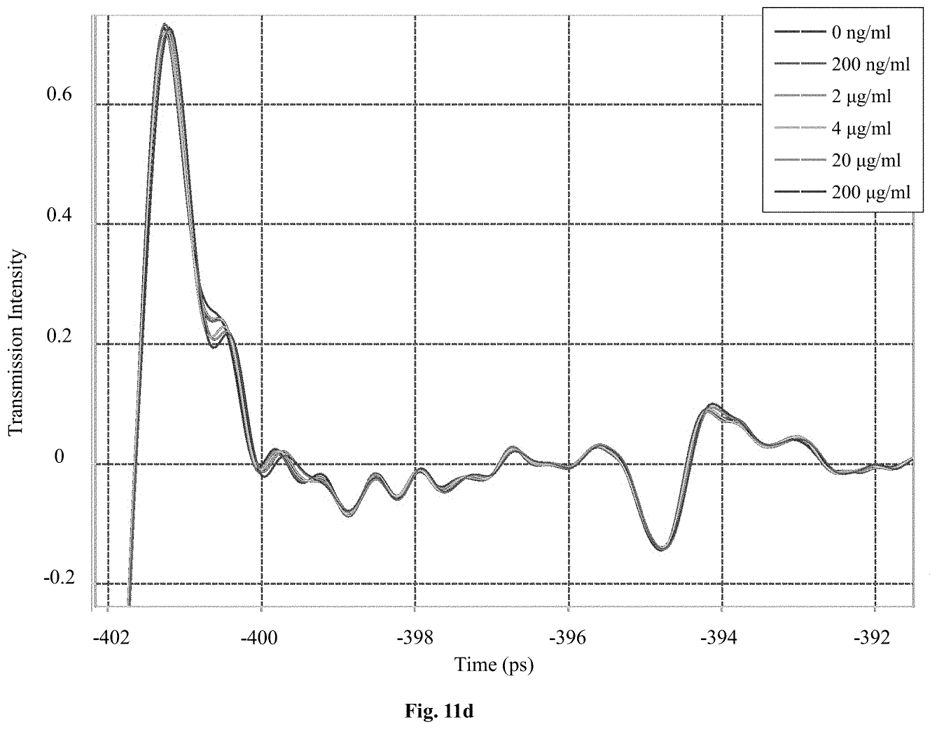

In order to evaluate the particle detection limit of the sensor chips of the present invention containing split-ring resonators nanoantenna operating in a pure LC mode, a proof of principle study was conducted in a lab, first using antibodies and binding proteins. Reference is now made to FIG. 11a showing the raw FFT in transmission power mode for different Anti-Cardiac Troponin I antibody (antiCTn1) concentrations, where the antibody particles have an overall diameter approximately 4 nm. A change in the absorption spectra becomes evident and visualised better on the expanded view between 1.0-1.6 THz which is shown in FIG. 11b. A monotonous trend is observed in the position of the resonance as well as the extent of the absorption. FIG. 11c shows impulse response for the different antibody concentrations. FIG. 11d is the expansion of the spectra of FIG. 11c for visualisation of the resonance peak.

The resonance band becomes shallow as the concentration increases, i.e. as the antenna structures begins to saturate with the antibody particles (about 3500 particles in every split-ring resonator nanoantenna in the array), the resonance becomes significantly damped. When there is no antibody on the surface (reference measurement at 0 ng/ml), the resonance peak is around 1.3 THz and has the highest absorption. As the concentration increases, the position of the resonance peak shifts to the left and the amplitude goes down. The lowest concentration of 200 ng/ml that is tested corresponds to about 7 antibodies/m².

Thus, the accumulation of particles in the split-ring resonators causes a change in the effective dielectric permittivity of the resonant devices, which causes the resonance position to shift away from its reference value. These trends demonstrate that the sensor can detect 4-nm sized particles at concentrations as low as 200 ng/ml. These particles were deposited over an area of 36 mm² and correspond to about 40 particles in every split-ring resonator nanoantenna in the array (sensing area per one nanoantenna is 10-15 μm²). The demonstrated responsivity of the sensor to particles of this scale at such low concentrations suggests that the sensor is well suited to detect biological particles such as viruses, which are typically larger in size (for example, the shell diameter of the SARS-CoV-2 particle varies between 70-140 nm).

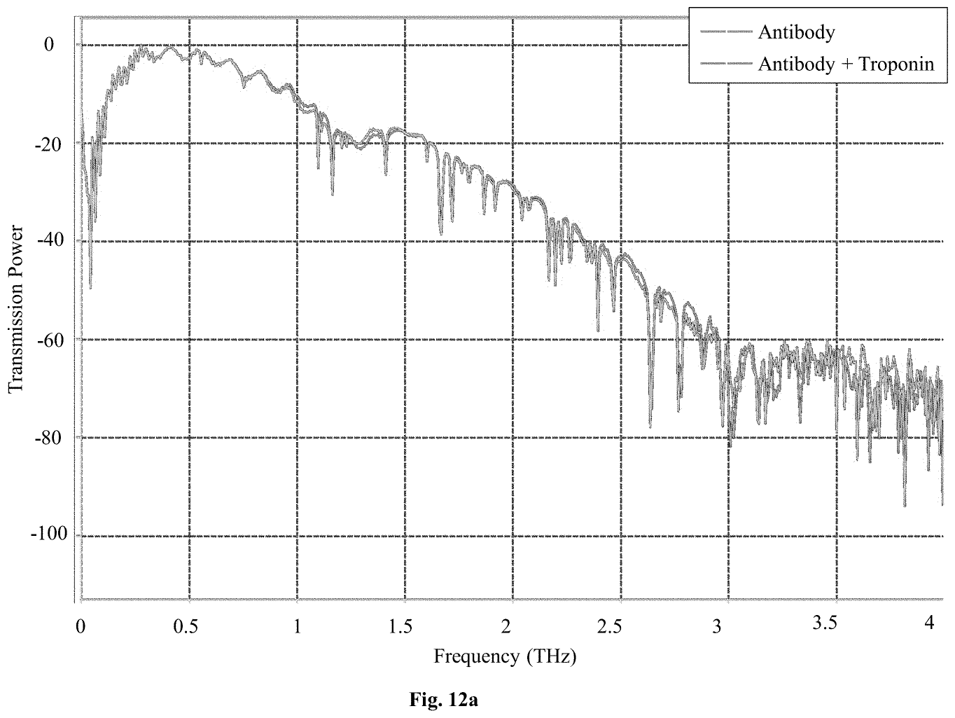

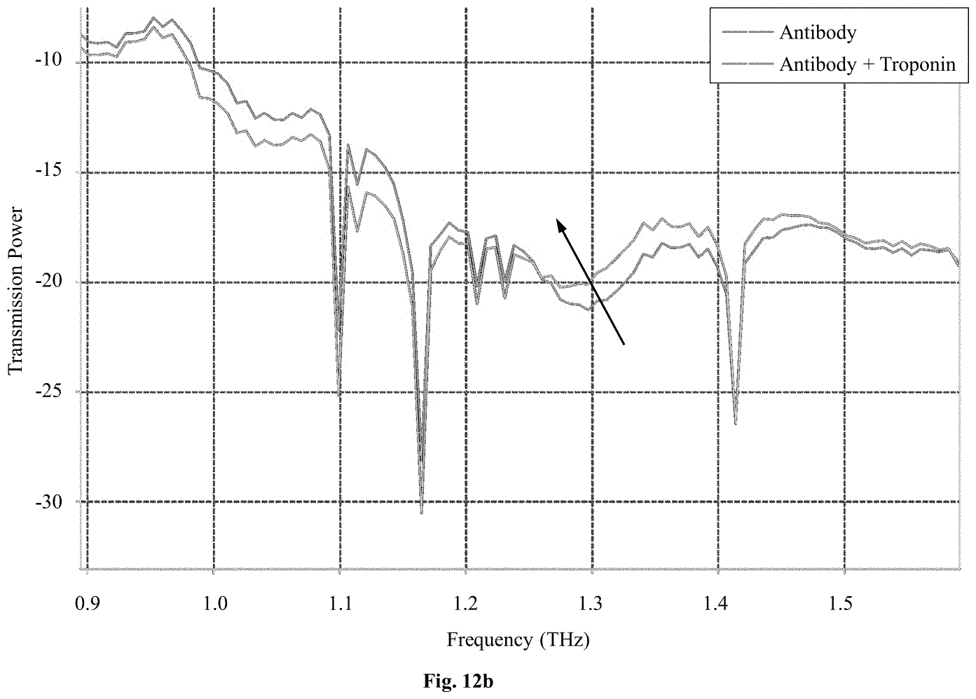

In order to test the sensor sensitivity, the antibody was mixed with the same concentration of troponin which binds to it. Reference is now made to FIGS. 12a and 12b that show the transmission power spectrum and its expansion around the resonance peak, respectively, for 2 mg/ml antiCTn1 (cyan line) and the mixture of 2 mg/ml antiCTn1 (green line) with troponin (wet). It is clearly seen from these figures that the resonance peak of the antibody mixture with troponin shifts relative to the pure antibody with additional damping in the absorption signal. The shift in frequency and change in amplitude of the resonance peak is observed around 1.3 THz when the antibody is bound to troponin. The overall size of the combined troponin and antiCTn1 is less than 10 nm. The slight increase in particle size and change in dielectric permittivity due to the binding results in a further shift in the spectral feature in the resonance region, but not at characteristics water lines in the vicinity of the resonance region, for example. This result clearly supports the conclusion that it is possible to detect interactions with the senor of the present invention on a molecular level.

Testing the Sensor with SARS-CoV-2 Virus Against Negative Control

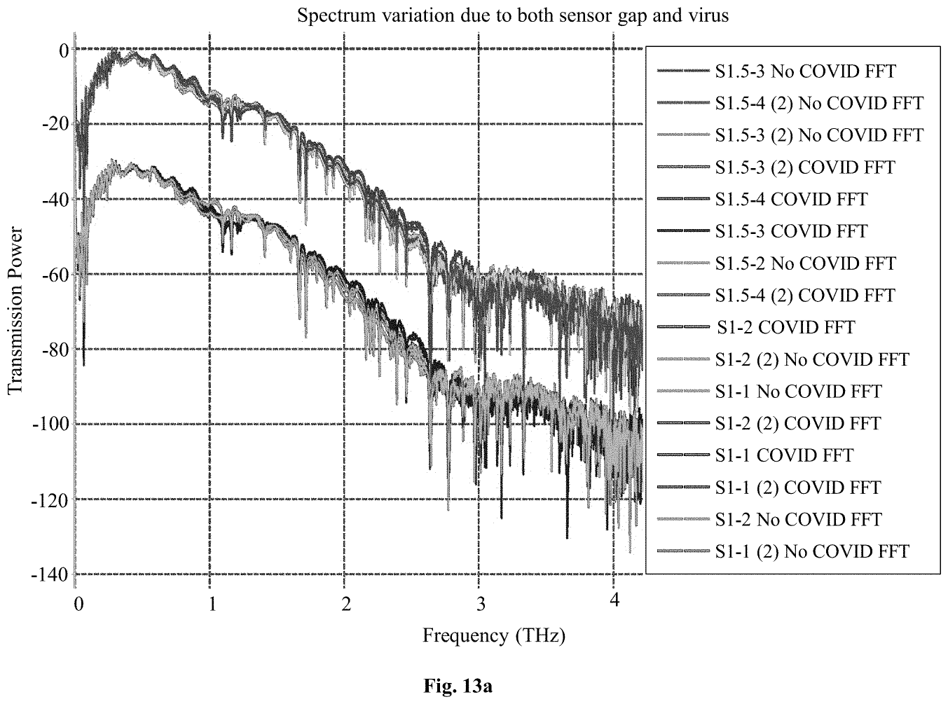

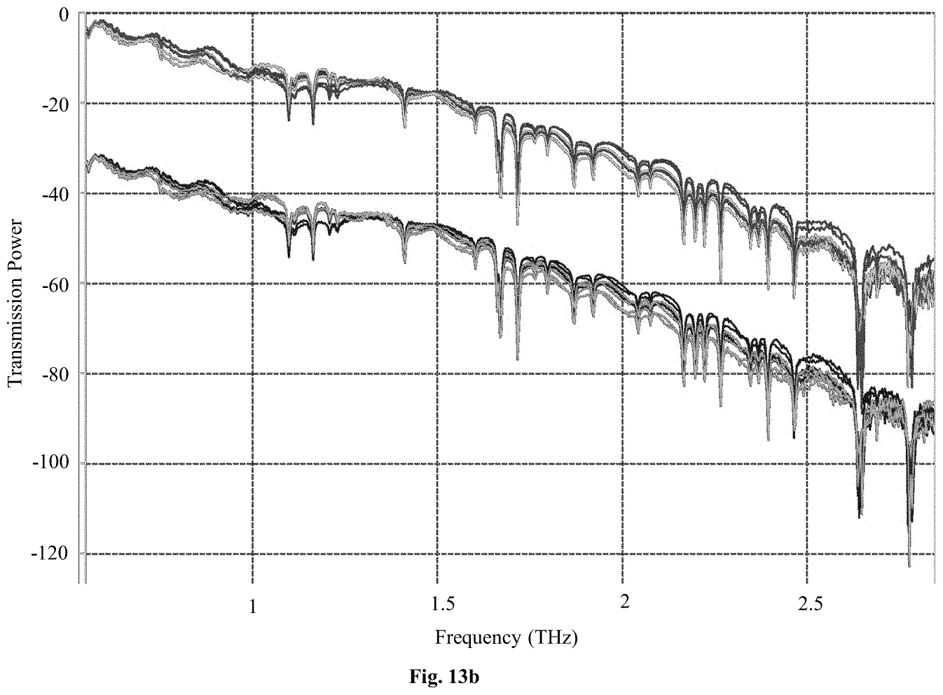

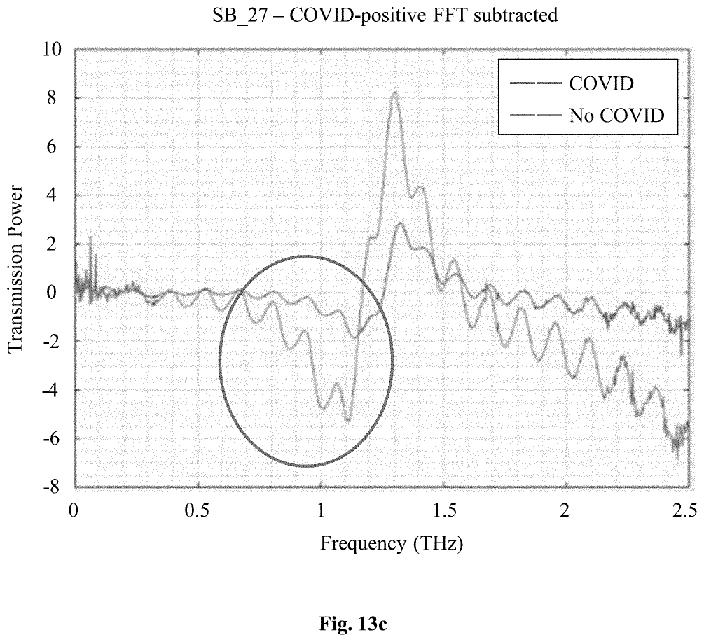

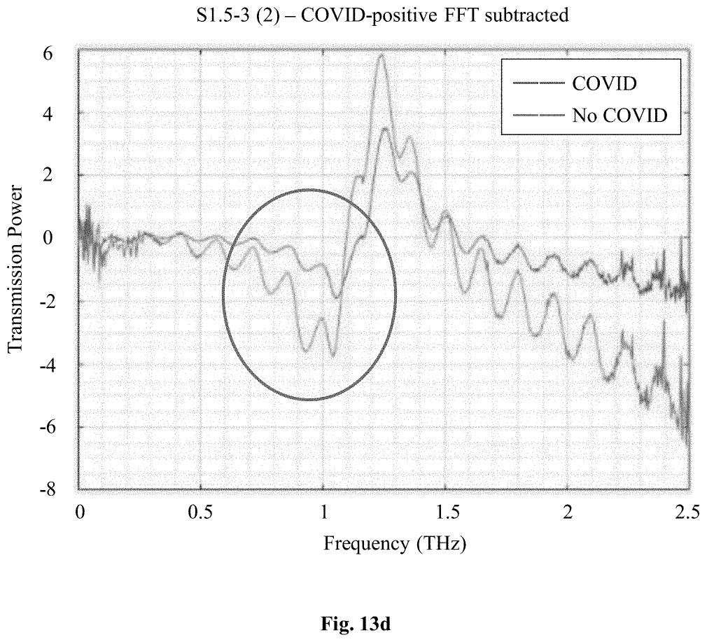

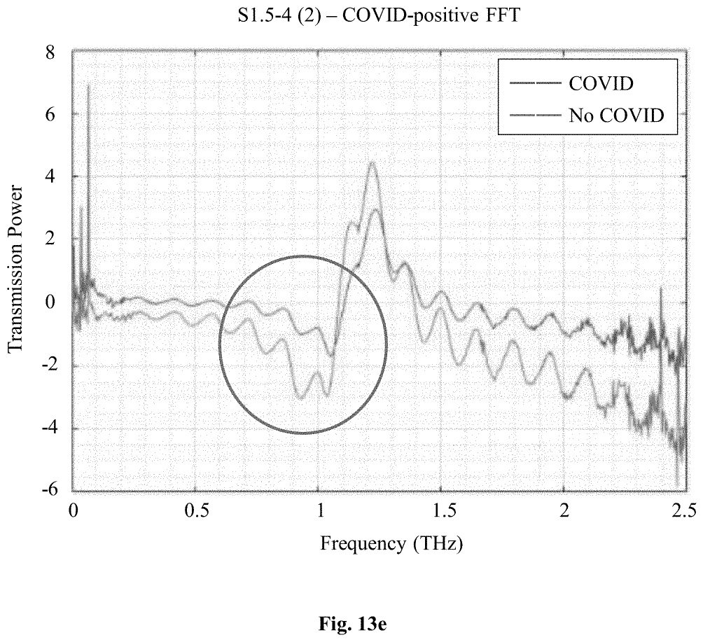

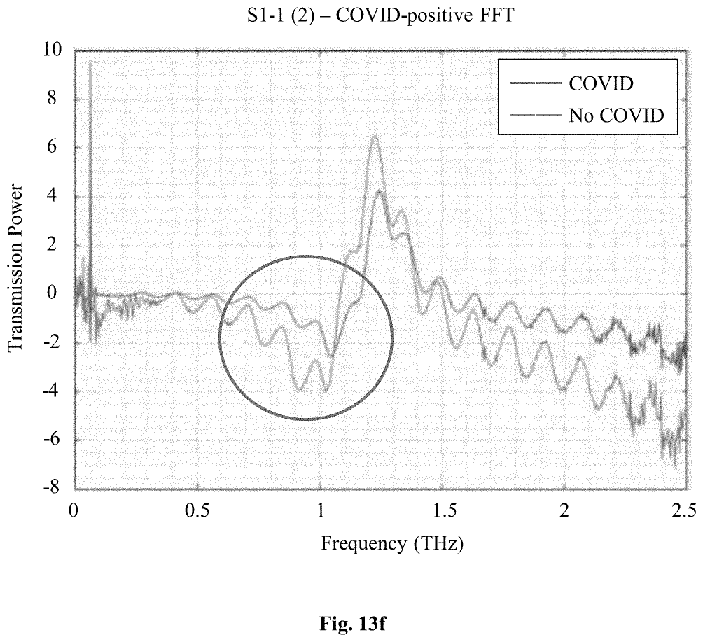

Reference is now made to FIGS. 13a-13f showing experimental results of the tests with the split-ring resonators nanoantenna structure as a sensing element operating in a pure LC mode on the SARS-CoV-2 infected samples versus healthy patient samples. Reference and sample FFT spectra (COVID positive and negative) were taken by reusing all five sensors in batches over the course of the experimental trials as described above. A general trend was observed across the entire dataset comparing positive and negative cases against the reference FFT spectrum in each case.

FIG. 13a shows a plot of the THz FFT spectra collected for all positive (dark blue and green colours) and negative (light blue and green colours) samples, with two different (blue and green) positive and negative samples, shown in dark and light colour schemes respectively. This plot gives an overview of the observed trends across the entire dataset which shows that the negative SARS-CoV-2 FFTs always shift further away from the reference compared to the positive SARS-CoV-2 FFTs. There is a clear distinction between dark and bright colours (positive and negative samples) around 1.2 THz. The negative samples consistently have a lower amplitude in resonance and a higher shift in frequency. To see this more clearly, the dataset is zoomed in FIG. 13b around the shift in the frequency range 1-3 THz. Based on the results shown in FIG. 13a-13b, the inventors discovered that across the entire data set, the negative COVID samples have a consistently lower amplitude in the resonance band. In addition, the negative COVID samples exhibit a higher frequency shift compared to COVID positive samples.

The experimental procedure for collecting the data shown in FIGS. 13a-13b is as follows. The THz spectra of air, the substrate (quartz) and nanoantenna were acquired prior to the measurement for baseline correction. Then 5 μl of SARS-CoV-2 positive solution was pipetted on the nanoantenna structure of the embodiments and dried. The drying was assisted by the ventilation in the fume hood. THz spectra was acquired in a transmission mode for a time interval of 200 ps and averaged over 500 times. The nanoantenna structure was then cleaned, and a reference spectrum of the nanoantenna structure was acquired to confirm that the baseline is achieved after cleaning. The same set of experiments were then done for control solution collected from healthy patients. The same set of experiments were carried out on three SARS-CoV-2 positive and three negative samples on multiple chips. Effects from temperature and humidity were adjusted because based on a reference data, and the position of the resonance peak prior to adding the sample was measured, thereby providing pure substrate (quartz) baseline data and nanoantenna data of the sample.

However, FIGS. 13a-13b also show that the sensor having the regular split-ring resonators structure operating in a pure LC mode cannot accurately differentiate between positive and negative samples. FIG. 13c shows the zoomed region in the THz FFT spectrum of the aforementioned sample SB_27, where the main region of difference for the positive versus negative samples is indicated with the circle. FIGS. 13d-13f show the zoomed peaks from FIG. 13b for three selected samples, where the same circle highlights the main region of difference for the positive versus negative samples. In FIGS. 13c-13f, blue lines represent the COVID-positive reference data, while red lines represent COVID-negative reference data (control data collected from healthy patients) for various samples mentioned above. These graphs demonstrate large difference in the amplitude between the THz spectra of the infected and negative samples and a consistency in obtained results.

Thus, the THz signal amplitude in the clean samples is higher compared to the infected samples, and the frequency is shifted. The collected data shown in in these spectra is subjected, for example, to a covariance matrix analysis to find maximum correlation in the spectra and to identify the regions of maximum difference between the samples in order to get a statistical distribution over a large set of data. As a result, the scatter plot is separated into positive or negative components.

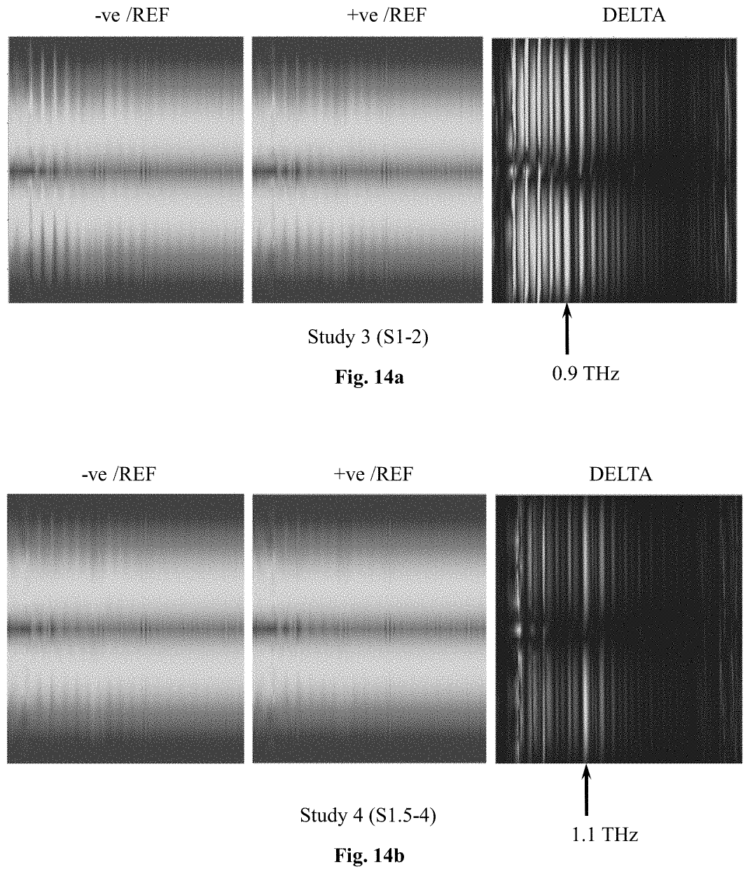

FIGS. 14a-14b show the exemplary cross correlogram maps demonstrating the correlation between the sensor with sample and sensor without any sample (reference) with a slicing window of 600 GHz moved over the entire frequency range. Axis X is a spectral band (THz), and axis Y is a frequency shift (THz). Left column shows the negative against reference and middle column shows positive against reference. Right column shows the difference (delta) between two cross correlograms. The frequency band near the resonance peak with higher correlation for positive shift (red colour on the top half) indicates higher correlation for positive samples to the reference spectra than negative samples showing that the SARS-CoV-2 virus has a higher impact on the effective refractive index of the medium.

In general, cross correlogram maps are used to identify spectral signatures across various normalised datasets. The aim of this analysis is to investigate differences between positive- and negative-COVID datasets. A cross correlogram map is a heatmap of the cross-correlation values between two identically shaped datasets. These values are plotted over the entire range of the independent variable for positive and negative shifts applied to one dataset relative to another. In the present invention, this operation was performed on the data in 600 GHz slices over the entire frequency range 0-3 THz (X-axis) for shifts up to ±60 GHz (Y-axis) in steps of 1.7 GHz.

The acquired FFT spectra, e.g. positive-COVID specimen were cross correlated to the THz biosensor's reference FFT with the above-mentioned moving windows. The result is the heatmap “+ve/REF” seen in the middle image in FIGS. 14a-14b. Similarly, the cross correlogram map for the negative-COVID sample is generated as seen in the right image in FIGS. 14a-14b (−ve/REF). These maps show highest correlation around zero shift, simply because they are similar signals, and the cross-correlation intensity reduces as the shift increases in either direction. However, the interesting part of this study is to observe how they differ in this aspect while comparing positive- and negative-COVID samples. This is done by taking the difference of the positive and negative cross correlograms (delta maps, taken as “+ve” map minus “−ve” map) shown in FIGS. 14a-14b, in the right image.

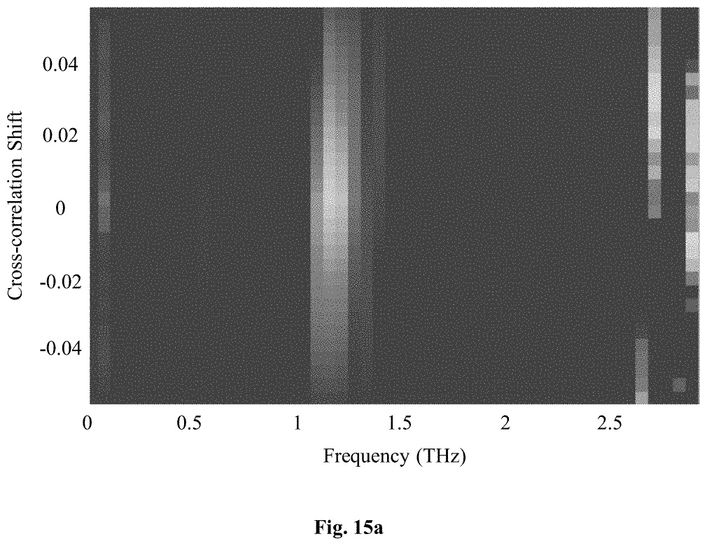

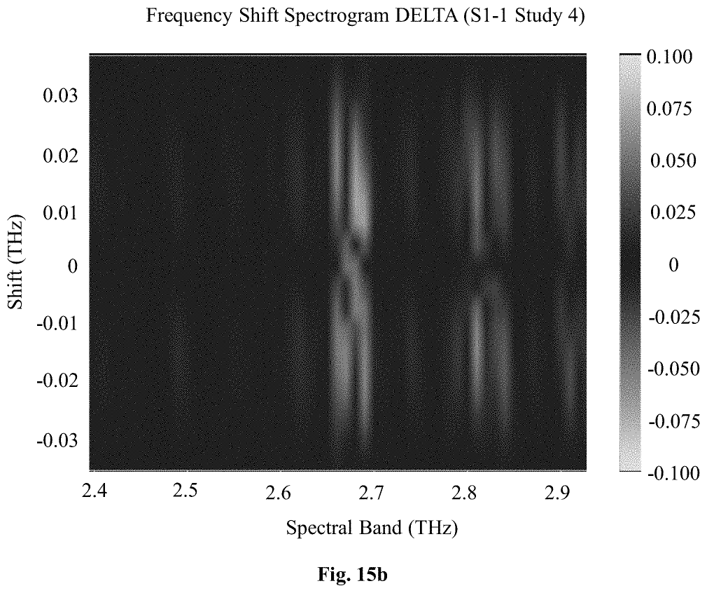

Reference is now made to FIG. 15a showing an example of such “DELTA” map using a different dynamic colour range to emphasise the main differences between positive- and negative-COVID specimens. There is a distinct change observed around 1.2-1.3 THz in this normalised cross-correlogram difference map between “+ve” and “−ve” sample spectra taken against the reference. This change indicates the resonance band of the nanoantenna. Additional changes are observed around 2.6-2.9 THz corresponding to water absorption lines. These changes are also interesting because they suggests there are differences in the way the water absorption occurs in the presence of the SARS-CoV-2 particles. This has to do with the way water molecules envelope the virus particles. Indeed, partial bonding of water molecules to the unique spike protein structure of the specific virus could lead to changes in selected absorption lines of water.

FIG. 15b shows the DELTA cross-correlogram between positive and negative samples at frequency between 2.4-2.9 THz. There are some correlation clearly observed around 2.6 THz, which is also corresponding to the water absorption spectra. Interaction of virus with water will leave these kind of ‘finger prints’ which can be used as a second form of validation increasing the accuracy of measurements with the sensor of the present invention.

### Asymmetric Metamaterials of the Present Invention

It is clear that the electric resonance metamaterials, such as those described in the articles by Park et al. (2017) and Ji-Hun Kang et al. (2018), are actually pseudo-metamaterials. They do not have any magneto-optic component in their response and are capable of sensing only inside the gap of their split-resonator rings. Any analyte molecule or particle not falling into this gap will not be sensed. Taking into account a very low stochastic probability of the analyte molecules or particles to fall directly into the gap (small particle size versus large split resonator area), it becomes clear that the prior art nanostructures cannot be used in sensing “dirty” samples without their pre-treatment and separation. That is why, Ji-Hun Kang et al. (2018), for instance, teaches drying virus liquid samples for a long period of time (at least 1 hour) in a controlled lab atmosphere (30% of relative humidity and 20° C.) to avoid any signal error from water absorption at the THz frequency. That is why, they propose growing many layers of a virus to fill in the gap to be able to measure the dielectric constant of the particular virus. It is clear that this procedure can only work with a single type of a pathogen or virus and not with complex “dirty” mixtures from the exhalation air containing a wide variety of different compounds including salts that strongly affect the measured capacitance of the sample. In fact, absolute permittivity of only true dielectric materials can be measured in the nano-gaps of the LC resonant circuits.

To sum up, all symmetric metamaterial structures, in which individual metamolecules have a symmetrical geometry, produce a “bright mode” in which the light is at normal incidence. The electric and magnetic field couple such that the magnetic field oscillation is completely reduced and only an electric field response is enabled. In the electric resonance metamaterials, the electric resonance will only respond to changes in the dielectric constant of the material, and will not show changes in the refractive index. Nevertheless, if the material's dielectric constant changes and also the refractive index changes, then this type of nanostructure will show a spectrum shift. However, this shift is not due to the change in refractive index, but only due to the change in dielectric constant. That is why, for symmetrical nanoantenna arrays which can operate only in the LC mode, the metamolecules will function only as classical capacitors, and the changes to the capacitor circuit will follow the classical inductor-capacitor circuit effect, thus measuring a change only in total capacitance inside the capacitor gaps. In other words, a nanoantenna based on the split-ring resonators is a pure LC-mode nanoantenna capable of measuring only capacitance and hence, recording only dielectric constant (permittivity) variations. This is true for most of the prior art works, including those mentioned above.

Such type of nanoantenna described, for example, in Park et al. (2017) is capable of measuring capacitance only inside the nano-gaps. It measures dielectric constant, which determines conductivity, of virtually anything coming into the nano-gaps, i.e., without any selectivity. In other words, the capacitance measured by the LC resonators is the total capacitance of everything that reached the nano-gaps, with absolutely no selectivity between different species. Therefore, the only way to show the THz spectra of a virus, for example, in Ji-Hun Kang et al. (2018), was to purify the sample, to remove all impurities, to dry it and deposit the dried and clean sample containing mainly the virus onto the nanoantenna surface. It is clear that the LC split-ring resonant circuits described in prior art, such as Park et al. (2017) and Ji-Hun Kang et al. (2018), are not capable of sensing “dirty” (non-purified) samples, such as samples taken directly from breath containing numerous compounds of organic and inorganic nature. Moreover, the LC split-ring resonators have predominantly the bright light emission mode which couples directly into space above the surface of the nanoantenna resulting in great losses and being capable of indicating only amplitude changes, with no frequency shift.

In contrast, asymmetric metamolecules (breaking symmetry) produces a dark mode coupling into a substrate, while at the same time exhibiting a bright mode, which also present in the spectra. Usually under the correct geometry, the spectra will have two resonance peaks (originated from both, bright mode and dark mode). In most cases, the bright mode coupling into space will have a larger peak in the THz spectra, while the non-radiational dark mode coupling into a substrate will have a small depth peak. However, the dark mode peak will have a much higher Q-factor and much more sensitive to refractive index changes, which opens the way not only to sensitive detection of analytes, but also to their specific identification. The nanoantenna structure of the present invention operating in both bright and dark modes and therefore, exhibiting two peaks as well as a frequency shift in the THz spectra will be described below.

In still another aspect, the nanoantenna of the present invention has a toroidal split-ring resonator shape of the metamolecules. The periodic array of the toroidal metamolecules used herein is defined as an “Aharonov-Bohm antenna”, which can detect signals in the THz frequency range. In general, a separate class of metamaterials is the one with the toroidal response. The toroidal observation is mediated by the excitation of currents flowing in inclusions of toroidal metamolecules and resembles poloidal currents along the meridians of gedanken torus. The destructive interference between the toroidal and electric dipole moments leads to lack of the far-fields but the fields in the metamolecules origin describing by S-function. Such field configuration, referred as a toroidal dipole or anapole, corresponds to the field of a solenoid bent into a torus and allows observing the phenomenon of electro-magnetically-induced transparency, provides an unusually high Q-factor in metamaterials, enables a cloaking for nanoparticles, and confirms the dynamic Aharonov-Bohm effect. Basharin et al in “Extremely high Q-factor metamaterials due to anapole excitation”, Phys. Rev. B 95, 2017, 035104, demonstrated that the anapole excitation in planar metamaterials, which enabled an extremely high Q-factor in microwave, gives promising opportunities for tuneable metamaterials due to the strong electromagnetic fields' localisation within metamolecules.

By applying additional plasmonic filters of any kind, the frequencies could be tuned precisely to a specific frequency of choice. In general, the present Aharonov-Bohm antenna is a co-planar broadband-antenna, which is made from a metamaterial in a form of multiple toroids. It is a reciprocal device and collects in a passive mode exactly the same frequencies that can be actively radiated. Due to its broadband character, the Aharonov-Bohm antenna may receive signals in a broad range between 30 GHz to 300 THz. The antenna is placed on a thin dielectric substrate composed for example, from glass, silicon or quartz. Advantages of the Aharonov-Bohm antenna is its broadband characteristics suitable for ultra-wideband signals in the THz frequency domain, its relatively easy manufacturing process using common manufacturing methods, and its easy impedance matching to the feeding line using microstrip line modelling methods. Also, the Aharonov-Bohm antenna has been chosen because it permits to integrate a long meander delay without having undesired effects.

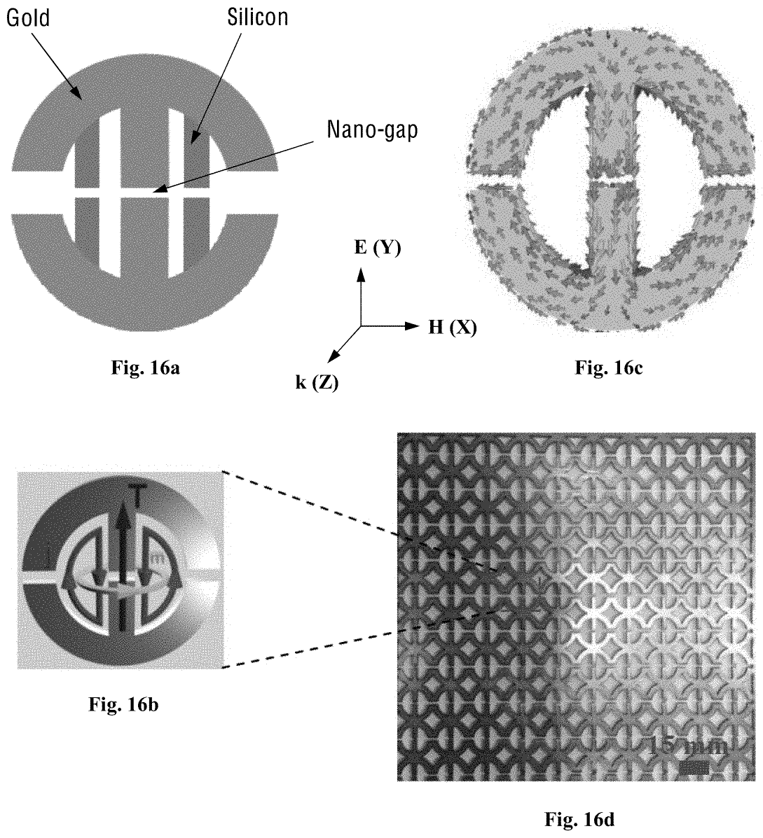

As mentioned above, the Aharonov-Bohm antenna of the present invention is based on the design of the toroidal metamaterial suggested by Basharin et al (2017) in a tuneable regime. The toroidal metamaterial consists of toroidal metamolecules made of photoconductive silicon capable of transiting from dielectric to metallic state and having the response in the sub-THz and THz frequency domain. As shown in FIGS. 16a-16c, the metamolecules constituting the toroidal metamaterial, shown in FIG. 16d, contain two split parts.

The incident plane electromagnetic wave with electric field E aligned with the central wire excites conductive currents in each loop of the metamolecule. The currents schematically shown in FIGS. 16b-16c form a closed vortex of magnetic field H. Each current induces the circulating magnetic moments m wreathing around the central part of the metamolecule. Such configuration of electro-magnetic fields supports the toroidal dipole excitation with a toroidal moment T as a result, oscillating upward and downward within the metamolecule along the Z axis. However, two side gaps also support a magnetic quadrupole moment Q. Moreover, due to the central nano-gap, electric moment P can be excited in the metamolecule, the electric dipole also arises in the metamolecule and maintains the anapole mode, and the central nano-gap becomes a necessary part of anapole in accordance with the destructive interference between electric and toroidal dipole moments. The advantage is a very narrow line in the transmission spectrum of a metamaterial. Based on this, the planar toroidal metamaterial is used as a building block for THz modulators in the present invention.

“Surface plasmon polaritons” (SPPs) are electromagnetic waves that travel along a metal-dielectric or metal-air interface. This term explains that the wave involves both charge motion in the metal (“surface plasmon”) and electromagnetic waves in the air or dielectric (“polariton”). They are a type of surface wave, guided along the interface in much the same way that light can be guided by an optical fibre. SPPs are shorter in wavelength than the incident light (photons). Hence, SPPs can have tighter spatial confinement and higher local field intensity. Perpendicular to the interface, they have sub-wavelength-scale confinement. An SPP will propagate along the interface until its energy is lost either to absorption in the metal or scattering into other directions (such as into free space).

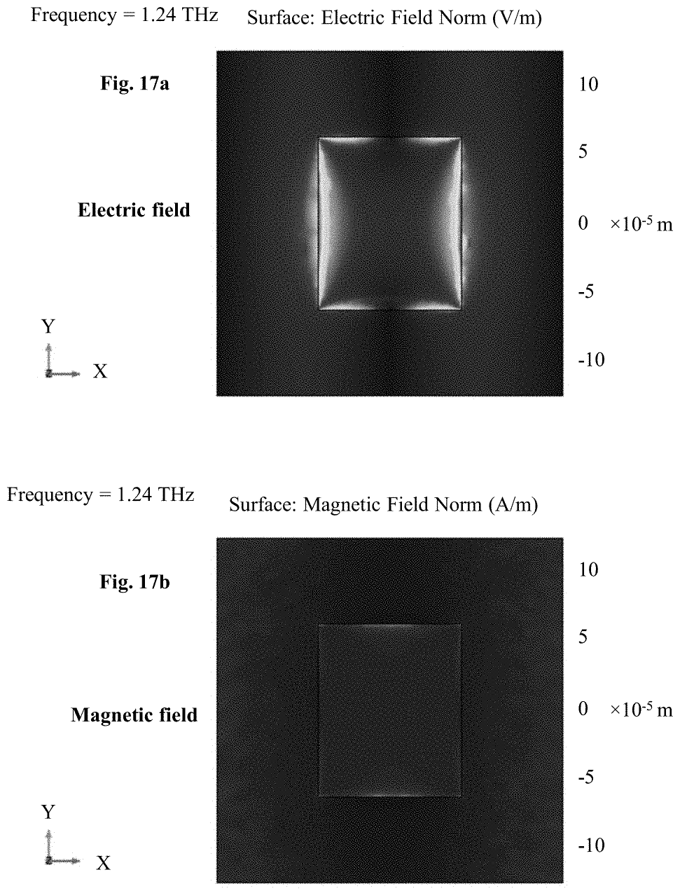

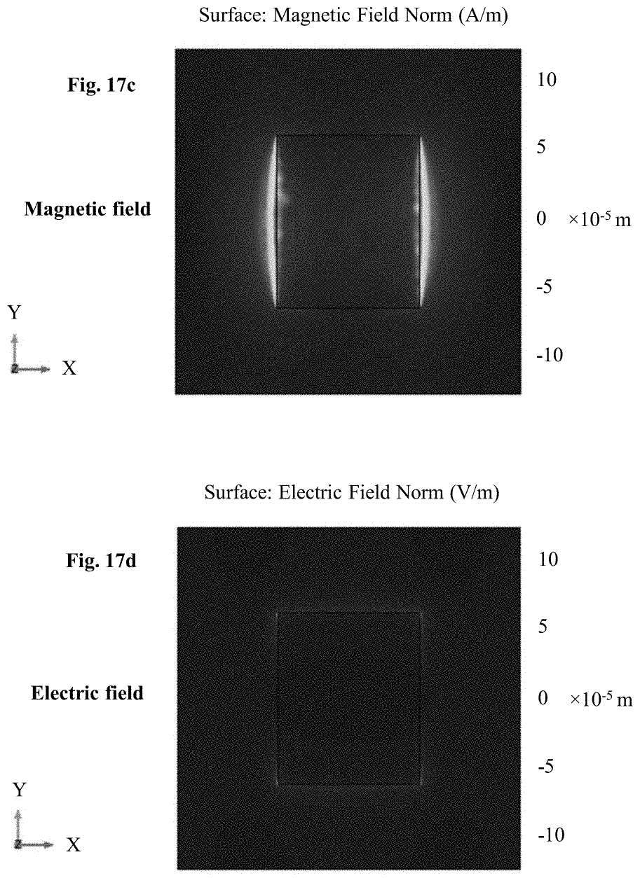

There are two types of SPP modes: electric and magnetic. When a metal film is used over the entire metamaterial surface and holes are cut in the metal film, an electric SPP wave is produced, because the charges are able to move through the metal film and the charge carriers interact between the cut-out holes in the metal film. FIGS. 17a-17b shows an example of this electric mode. As can be seen, only the electric field in FIG. 17a is active, and the magnetic field in FIG. 17b is not active.

When there is no metal film covering the entire sensor surface, but instead metal structured metamolecules cover the surface, then with the right design and pitch, it is possible to generate an SPP effect. However, this SPP effect will be magnetic, and it will be the magnetic field that is interacting between the metal-structured metamolecules. This can be seen in FIG. 17c, where at the main SPP frequency only the magnetic field is active, while the electric field in FIG. 17d is almost completely inactive. This is an important point, because in order to use the SPP effect to observe a near field interaction with a target analyte, a strong electric field must be created that will interact. It means, a way to induce an electric mode in the SPP should be designed while still using metamolecules that are metal islands and not cut-outs or holes in a metal film. This is very important in order to have a strong interaction between the absorption and emission of a target analyte.

It is possible to enable magnetic field interactions in the metasurfaces comprising split-ring resonator metamolecules by modifying the structure of the latter. In one embodiment, the nanoantenna structure of the present invention comprises an array of metamolecules, wherein each metamolecule is composed of two square split-ring resonators and a single metal bar over these two resonators. Reference is now made to FIG. 18 schematically showing the nanoantenna structure of the present invention. The bar over the resonators acting as a wave container is capable of exciting the dark mode in these resonators, followed by coupling it into the resonators. The distance between the bar and the resonators producing the maximum resonance effect is calculated and experimentally confirmed. This distance creates the so-called “electromagnetically-induced transparency” window (EIT) in THz band, which is a quantum interference effect resulting in sharp transmission window inside the absorption band of a three-level atomic system and therefore, making propagation of light through an otherwise opaque atomic medium possible. In other words, the EIT is a coherent optical nonlinearity which renders a medium transparent within a narrow spectral range around an absorption line.

Most metamaterials, such as graphene, exhibit the EIT phenomenon, observation of which involves two optical fields (highly coherent light sources, such as lasers), which are tuned to interact with three quantum states of a material. The “probe” field is tuned near resonance between two of the states and measures the absorption spectrum of the radiative transition. A much stronger “coupling” field is tuned near resonance at a different transition. If the states are selected properly, the presence of the coupling field normally creates a spectral “window” of transparency which will be detected by the probe. The coupling laser is sometimes referred to as the “control” or “pump”, the latter in analogy to incoherent optical nonlinearities such as spectral hole burning or saturation.

The nanoantenna structure of the present invention described herein exhibits the multi-band EIT effect due to the strong near field coupling between the bright mode of the bar and dark modes of the pair of the split-ring resonators. This configuration allows to independently tune the created EIT windows which is actually a very challenging task. The proper distance between the two resonators was calculated and then experimentally confirmed in order to avoid any cross-talk between them. Placing any obstacles, such as an additional metal bar between the first metal bar and the resonators, results in no further shift of the resonance peak in the THz spectrum. This is simply because the effect of the bar is purely magnetic with a polarisation wave propagating along the bar. To conclude, using the LC resonators without the bar will never allow specific, selective sensing. The nanoantenna structure of the present embodiment, with the waive container, demonstrated ultra-high sensitivity and specificity toward tested analytes, such as pathogens and viruses, in non-purified samples.

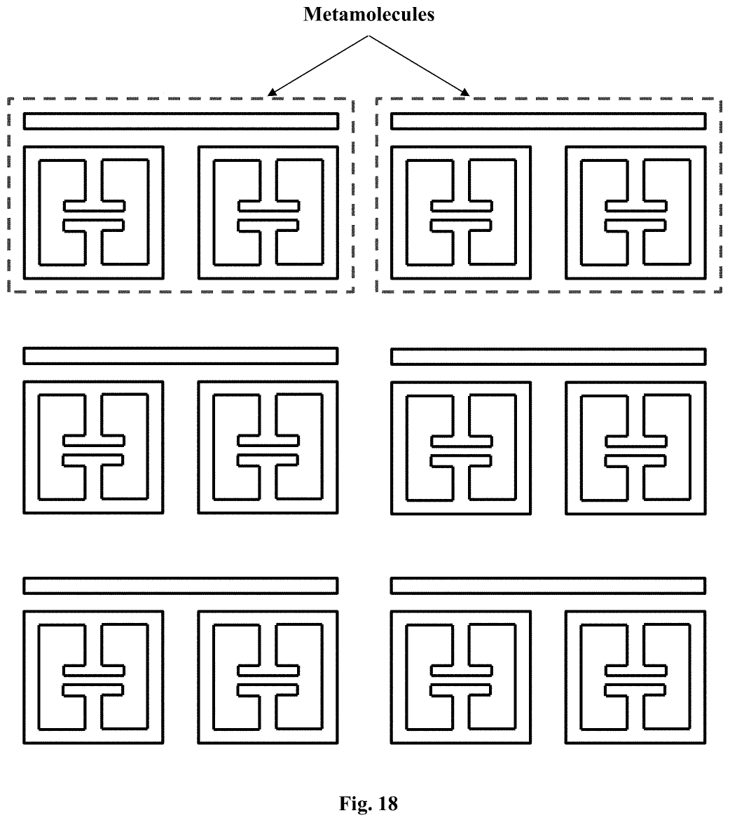

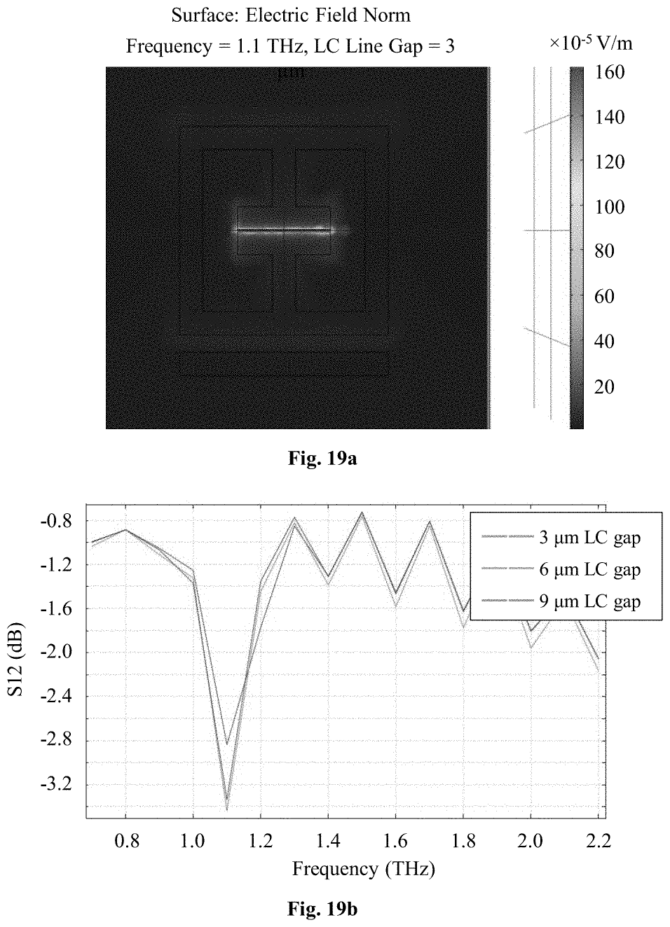

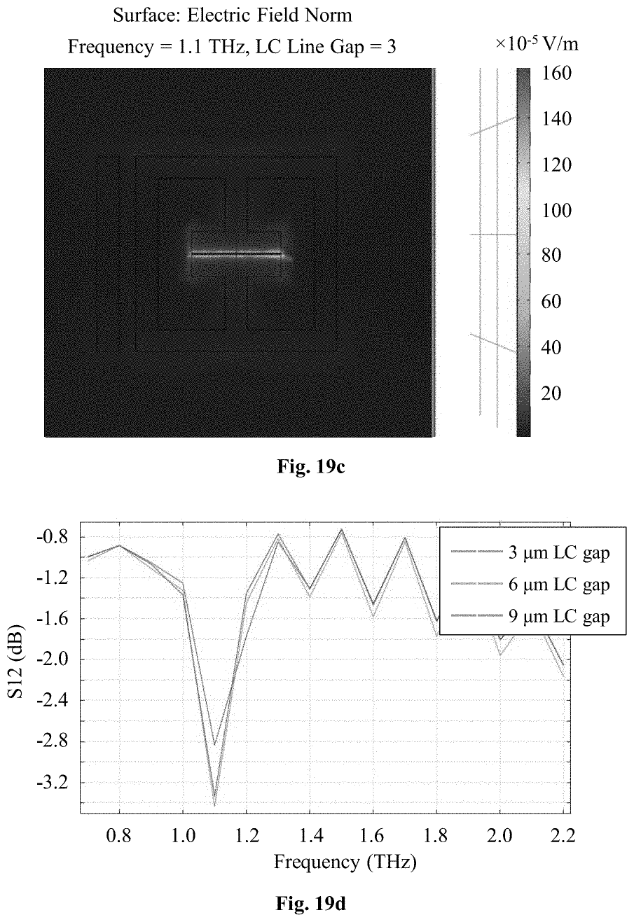

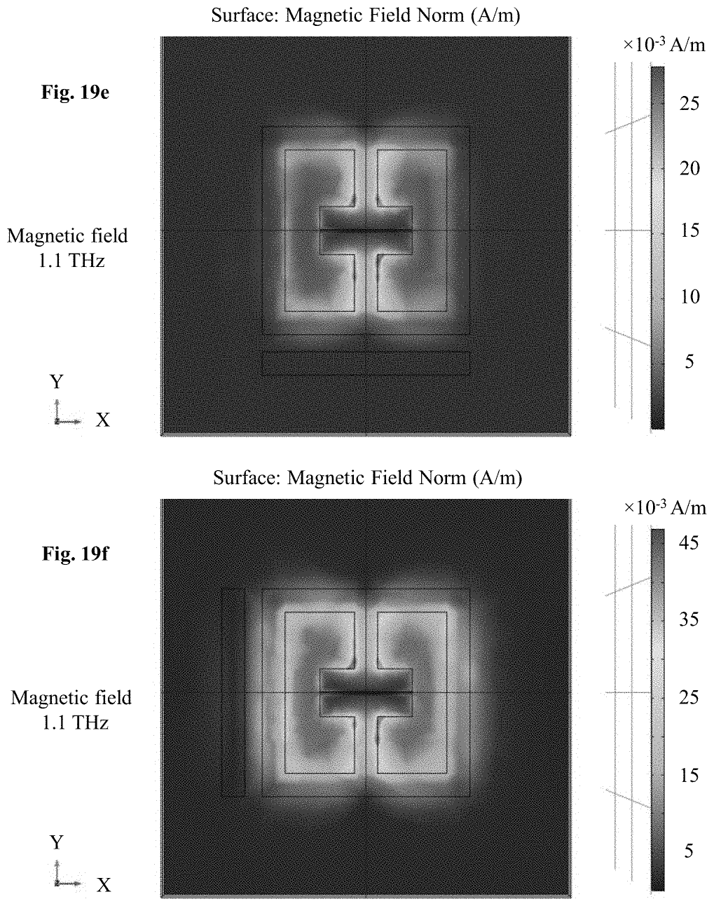

Reference is now made to FIG. 19a shows the comparative simulation of the electric field distribution in the individual split-ring resonator with a metal bar (cut wire) over it, placed in parallel to the gap, since the electromagnetic wave propagates along the gap. FIG. 19b shows the resonance spectra in the THz frequency domain. As clearly seen from these spectra, the change in the gap width from 3 μm to 9 μm does not produce any shift at the 1.1 THz resonance frequency. However, this is the only parameter that Park et al. (2017) and Ji-Hun Kang et al. (2018) suggest to tune in order to detect different viruses in this gap. FIGS. 19c-19d show the similar results when the metal bar is placed in a perpendicular direction to the gap. Again, there is no shift in the resonance frequency observed.

Thus, when using a single split-ring resonator structure with a metal bar to excite a “dark mode”, the EIT window cannot be created, since there is no destructive interference between the bright mode and dark mode. In addition, a strong magnetic excitation from the metal bar is required, which is not possible with a single split-ring resonator and metal bar. FIGS. 19e-19f show the magnetic field distribution in an individual split-ring resonator with a metal bar over it, placed in a parallel direction and in a perpendicular direction to the gap, respectively. As can be seen from these figures, placing a metal bar (cut wire) near the single split resonator, or even changing the polarisation direction of the e-field from X to Y (parallel to perpendicular) is surprisingly not enough to create the EIT effect and to form the EIT window, since the magnetic field is not produced by the metal bar.

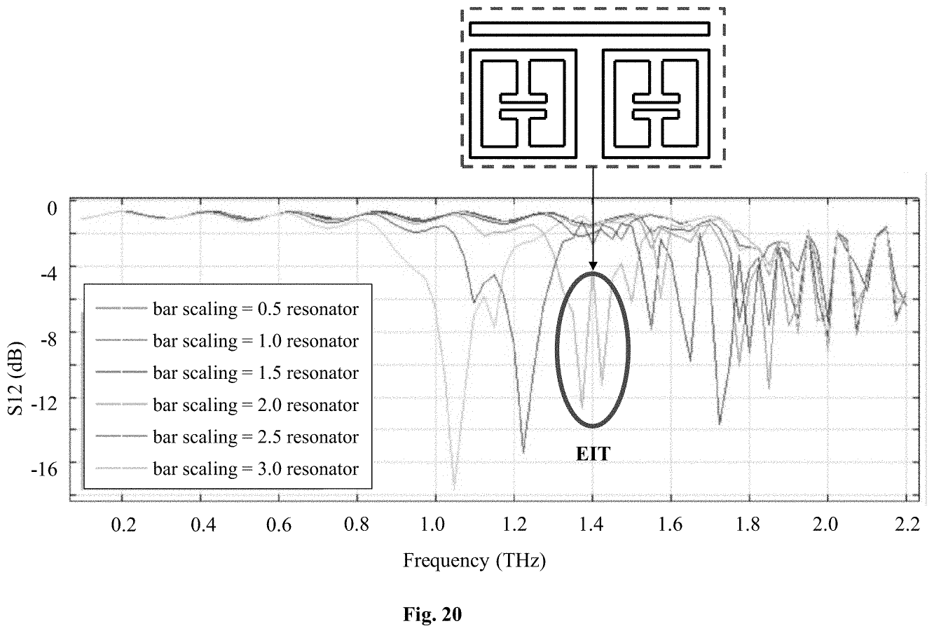

Reference is now made to FIG. 20 showing the comparative THz spectrum for different number (from 0.5 to 3.0) of the split-ring resonators in a single metamolecule. As can be seen, the metamolecule composed of two split-ring resonators and a metal bar over the resonators (cyan line) creates the best desired split (EIT) in the spectrum. Thus, for creating the EIT effect in this structure, a strong magnetic mode, which is a dark mode, should be produced by the metal bar and radiated into two split-ring resonators to create the clear EIT window.

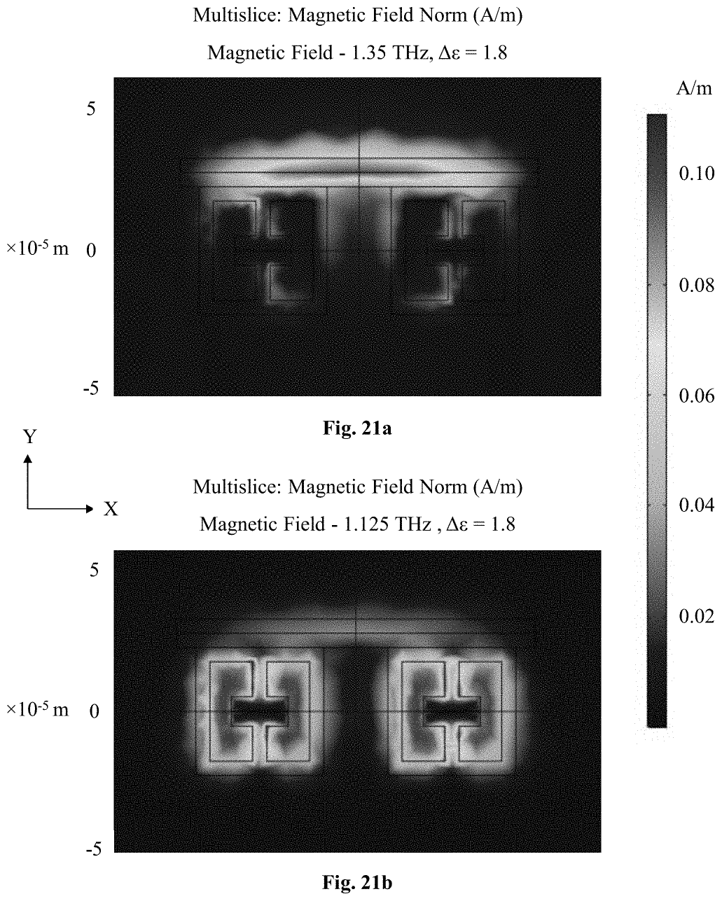

FIGS. 21a-21b show the simulation of the magnetic field distribution in the metamolecule composed of two split-ring resonators and a metal bar over them at 1.35 and 1.125 THz resonance frequencies, respectively. As can be clearly seen, there are two main magnetic modes appearing. FIG. 21a shows the dark mode which is produced by the metal bar and radiated into the split-ring resonators. This is actually a bouncing wave between the metal bar and the split-ring resonators, thereby producing the dark mode. This magnetic mode only appears between the lateral side of the two split-ring resonators and the orthogonal oscillations of the magnetic bar and only at a specific frequency of 1.35 THz. FIG. 21b shows the magnetic mode of the split-ring resonators themselves. It is observed at rather a different frequency of 1.125 THz and is not a part of the dark-mode produced by the EIT interaction between the metal bar and the resonators.

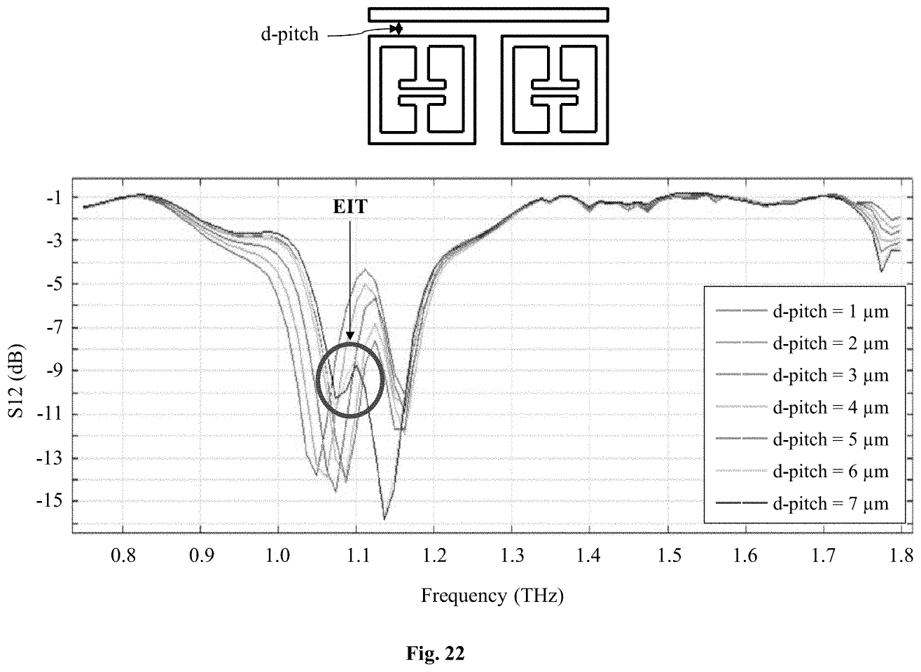

The explicit EIT window is observed in the THz spectrum in FIG. 22 due to excitation of the dark mode. As mentioned above, the bar over the split-ring resonators excites the dark mode in the resonators, followed by coupling it into the resonators. The distance between the two paired resonators was calculated to avoid any crosstalk between them. Also, the distance between the bar and the resonators, which is defined herein as “d-pitch”, producing the maximum resonance effect was calculated and experimentally confirmed. As clearly seen in FIG. 22, this distance creates the electromagnetically-induced transparency window (EIT) in the THz band around 1.1 THz. In fact, the correct d-pitch, distance between the two resonators and between neighbouring metamolecules can significantly amplify the EIT effect that enables radiative coupling, which can dramatically increase and affect the EIT window. If these distances are not correctly configured, the EIT window will collapse or vanish. For example, by changing the d-pitch as above, the EIT effect can be destroyed and the EIT window will disappear. This shows high sensitivity of the EIT effect to other fields, even if coming from adjacent (neighbouring) pixel units, and this is actually a key reason why the EIT window is so sensitive to any near-field or far field effects that are introduced into it.

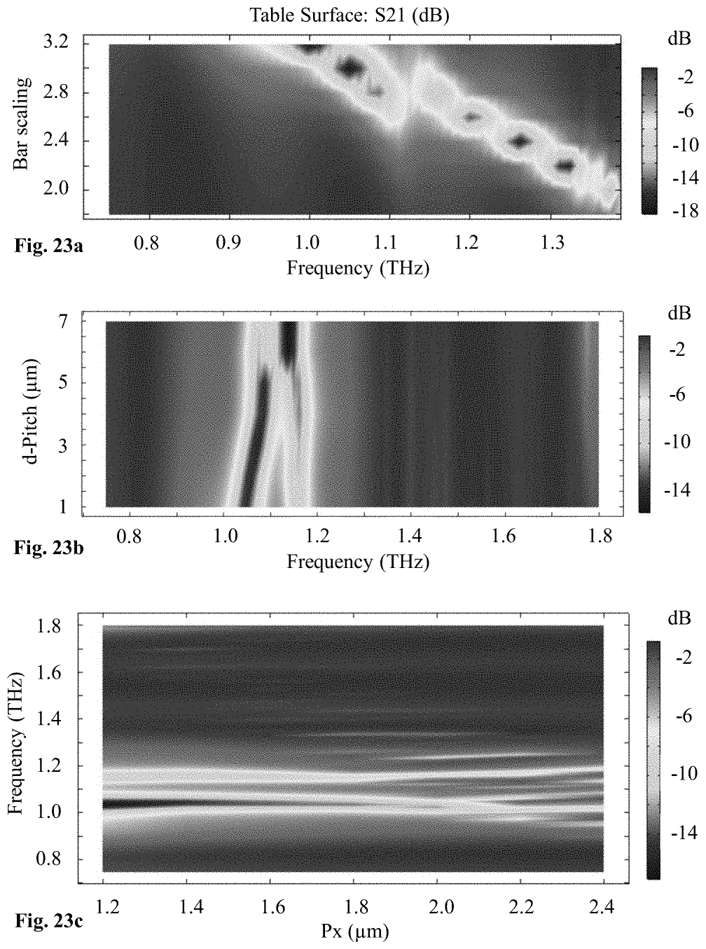

FIG. 23a-23c show the THz spectrograms of the nanoantenna structure of the present invention made of the metamolecules comprising two resonators and a metal bar. FIG. 23a shows how the EIT window appears and disappears based on the length of the metal bar (the blue regions are the EIT windows). FIG. 23b shows how the EIT window appears and disappears based on the distance between the metal bar and the resonators (d-pitch). FIG. 23c shows how the EIT window appears and disappears based on the distance (Px) between neighbouring metamolecules in the array.

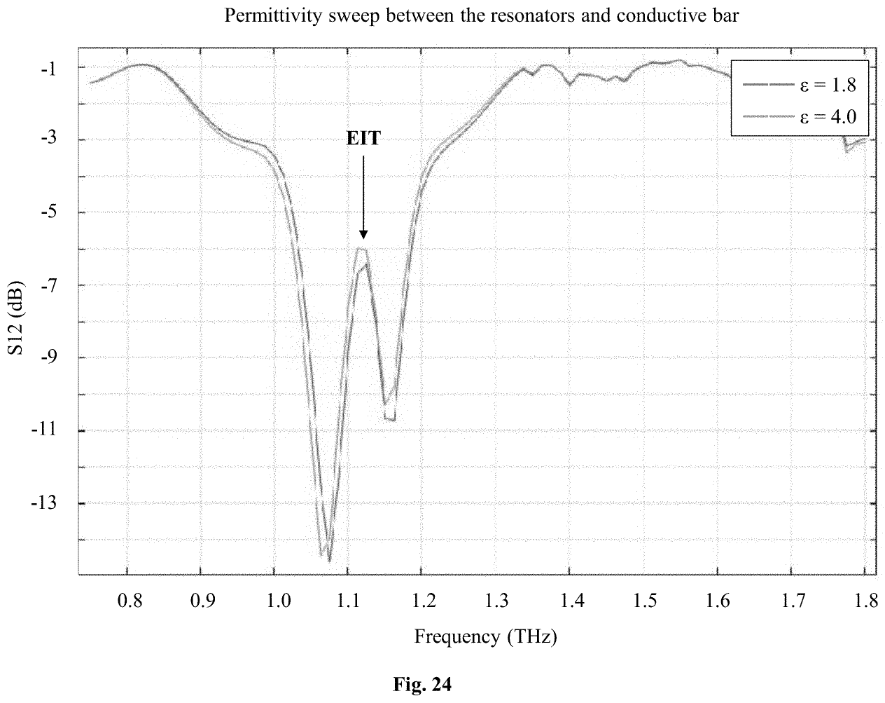

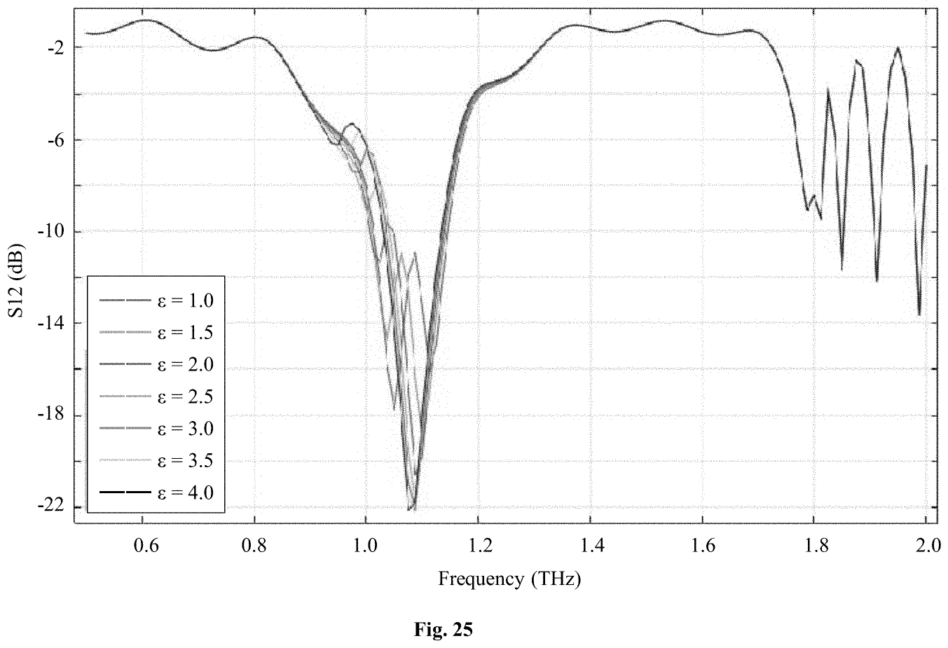

Reference is now made to FIG. 24 showing an optimised EIT window of the nanoantenna of the present embodiment for two samples having different permittivity (F), which is the function of a refractive index. These two samples having very different permittivity (F=1.8 and F=4.0) are placed between the metal bar and the resonators. As can be clearly seen, there is absolutely no change in the EIT window observed. This proves that the EIT window is only affected by the sensing area of the metamolecule and not by the magnetic mode near-field interaction between the metal bar and the resonators. FIG. 25 shows the effect of the analyte concentration in the resonator sensing area on the EIT window. As can be seen, the sensitivity of the EIT window to small changes in the resonators sensing area is huge, which can reduce the EIT window until it is completely gone.

To sum up, the nanoantenna structure composed of the nano-gap split-ring resonators acting as traditional LC circuits have the following drawbacks that make their practical application in sensing analytes almost impossible:
(a) Sensing analytes only inside the nano-gaps drastically reduces the overall sensing area of the nanoantenna structure and its sensitivity as a result;
(b) Measuring only total capacitance (dielectric permittivity) of a sample drastically reduces signal to noise ratio and selectivity of the sensor toward different analytes;
(c) Sensing only pre-treated, purified, separated and dried samples makes it impossible to perform rapid, point-of-care diagnostics; and
(d) Detecting only amplitude changes in the THz spectra drastically reduces selectivity of the sensor, making it impossible to distinguish between different analytes having similar shape and structure.

In another embodiment, the nanoantenna structure of the present invention comprises an array of metamolecules, wherein each metamolecule is composed of a spiral resonator and a metal ring surrounding and confining this resonator. This metal ring is also called “a wave container” or simply “container” as it contains the resonating electromagnetic field and keep it confined inside the ring. In other words, the ring is capable of exciting the dark mode in the spiral resonator, followed by coupling it back into the resonator. In addition, the spiral geometry of the nanoantenna provides extremely enlarged sensing area compared to other designs.

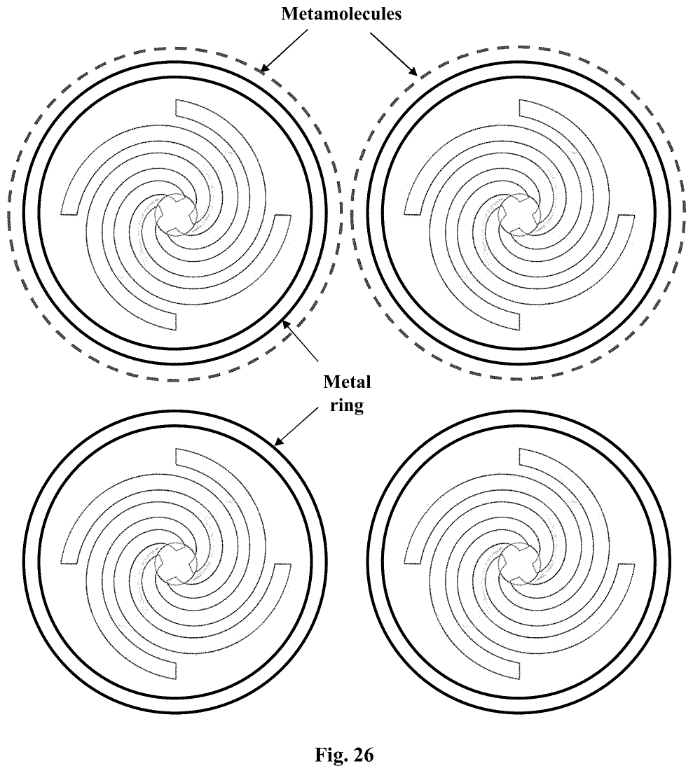

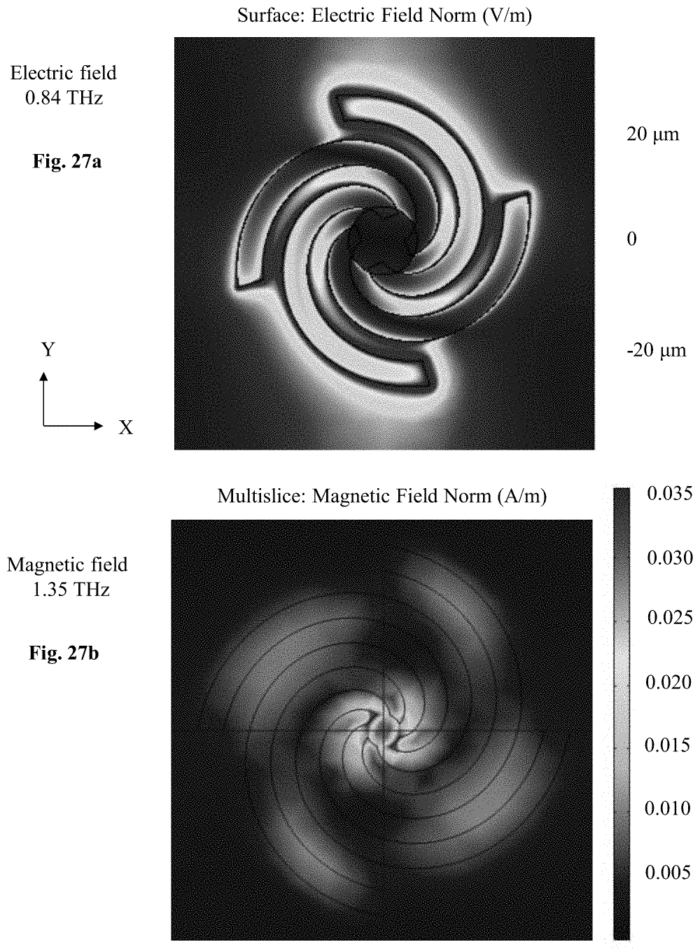

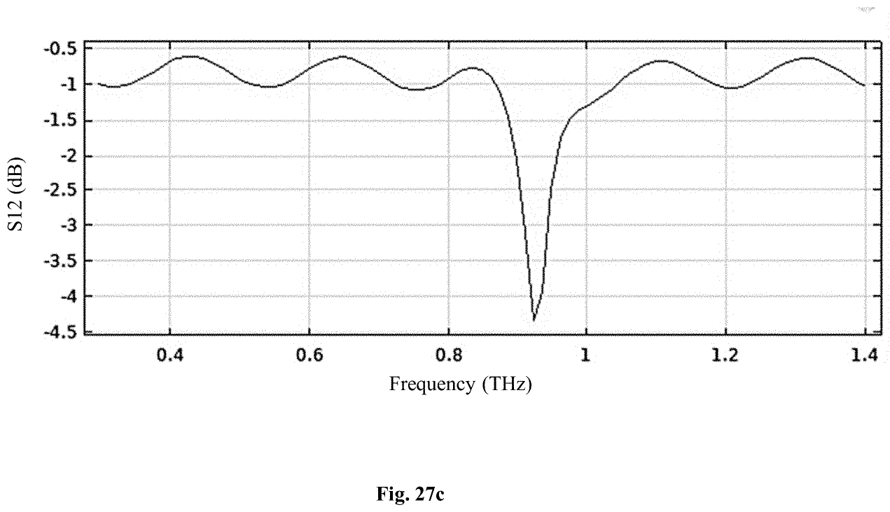

Reference is now made to FIG. 26 schematically showing the spiral nanoantenna array structure of the present invention. FIGS. 27a-27b show the electric and magnetic field distribution, respectively, inside the metamolecule without the container. FIG. 27c shows the spectrum of such spiral antenna without the container. As it can be seen, the spiral has only the bright mode (without the container) with no other modes formed.

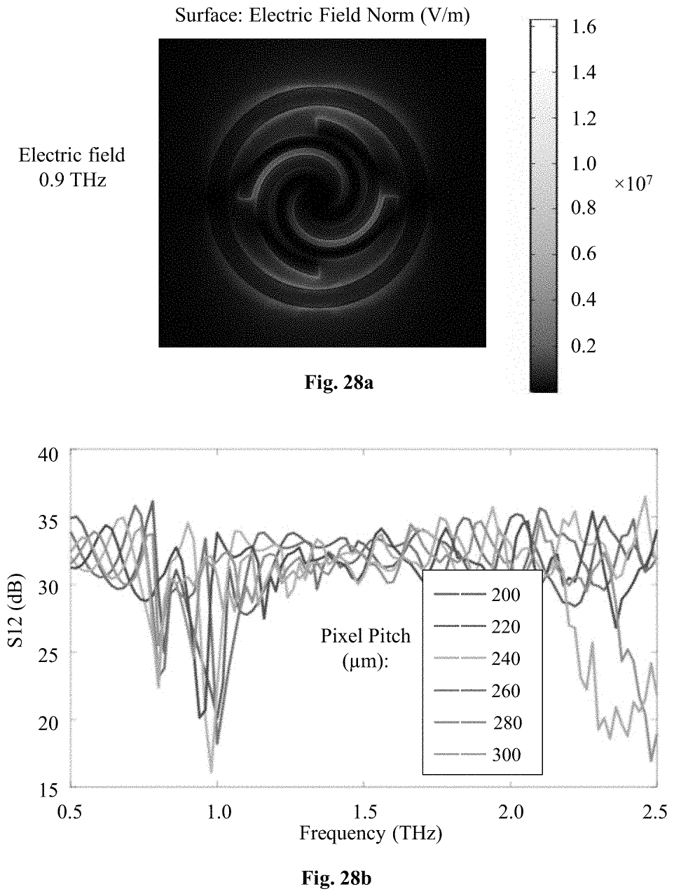

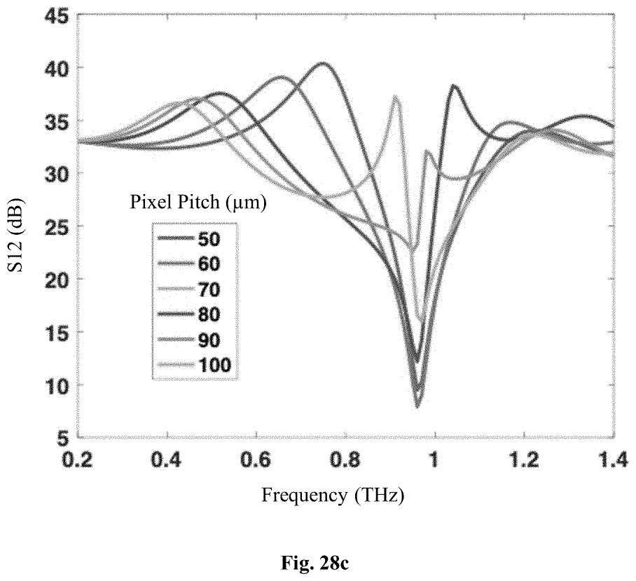

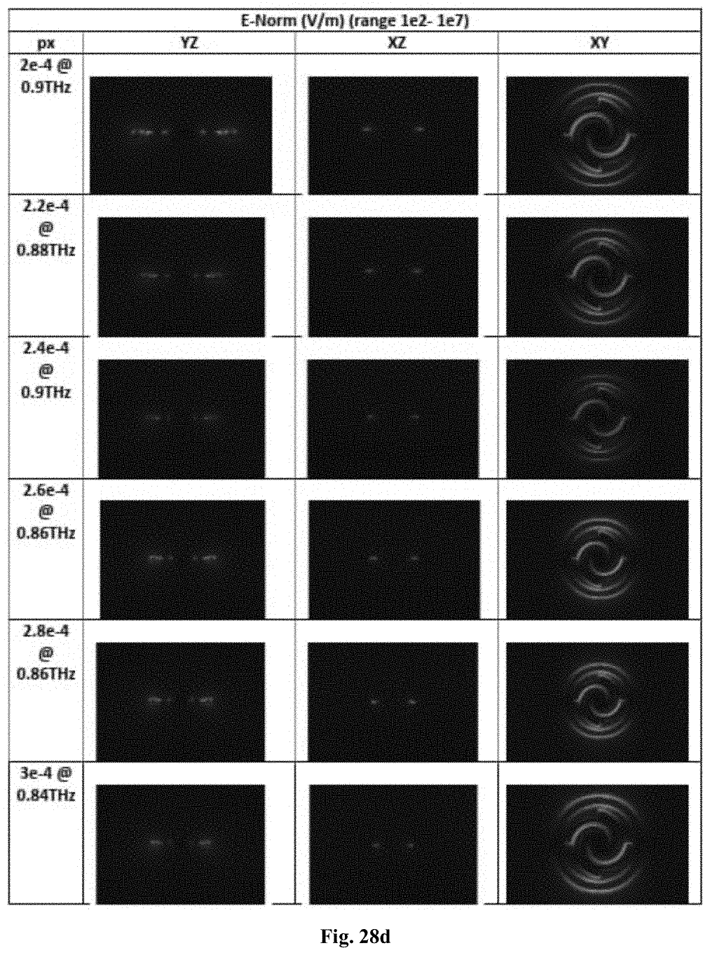

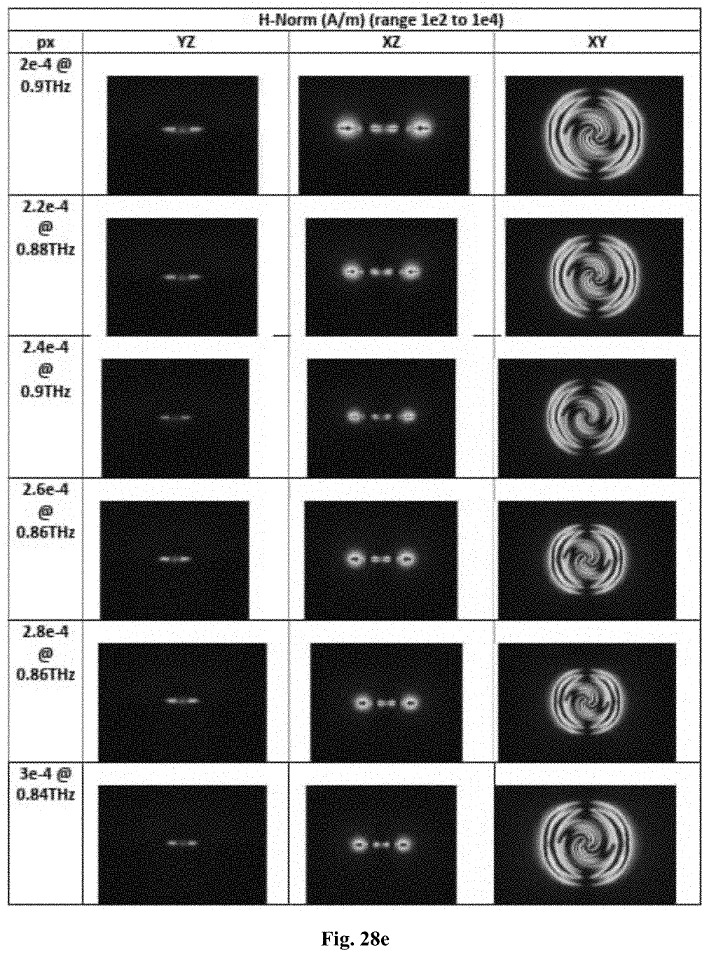

Adding the closed metal ring structure as a container makes it possible to create a shift in the frequency to a specific frequency for the bright mode, which is 0.9 THz in this case, thereby creating a dark mode at around 0.55 THz. FIG. 28a shows the dark mode of the electric field created at the 0.9 THz frequency. It should be noted that the bright mode here has also an SPP effect which can be shifted by changing a “pixel pitch”, which is defined as a distance between the spiral and the ring (shown as different coloured curves from 200 to 300 m). FIG. 28b shows the S21 spectra for different pixel pitches. FIG. 28c shows the S21 spectra for different pitches demonstrating that the change of the pitch in the direction of the electric field will collapse the main bright mode as it has a strong SPP influence and will also reduce the dark-mode. Changing the pitch in the direction of the h-field will not collapse the overall dark and bright mode, but will shift the frequency and shape of the resonance. It means this effect is very much a magnetic SPP effect, and the extra dark mode is produced by the magnetic field radiating into the sensing spiral. FIGS. 28d and 28e summarise these effects for various pitches for magnetic and electric fields, respectively, in different wave propagation directions. Thus, adding the container that creates both a dark mode and the SPP allows to bring the SPP effect to and enhance surface detection in any sensing structure, such that the final structure will have the SPP, dark mode and bright-mode.

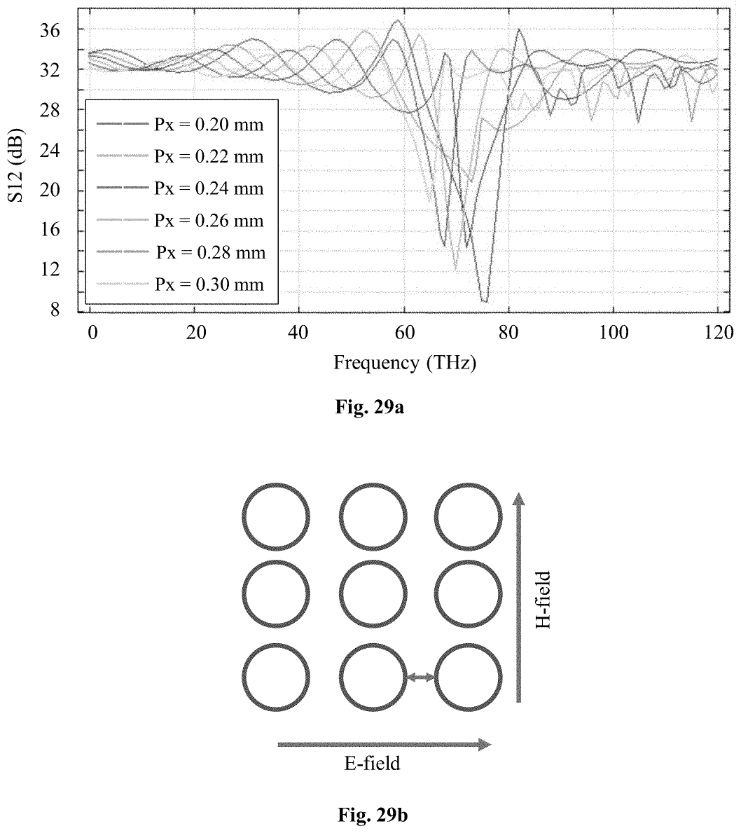

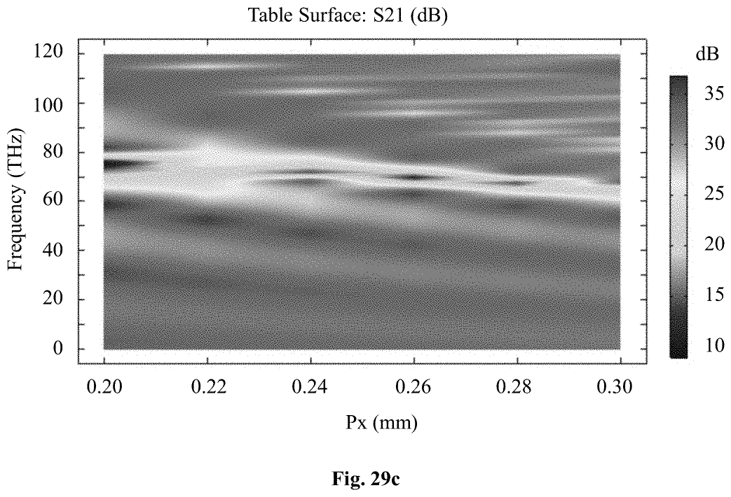

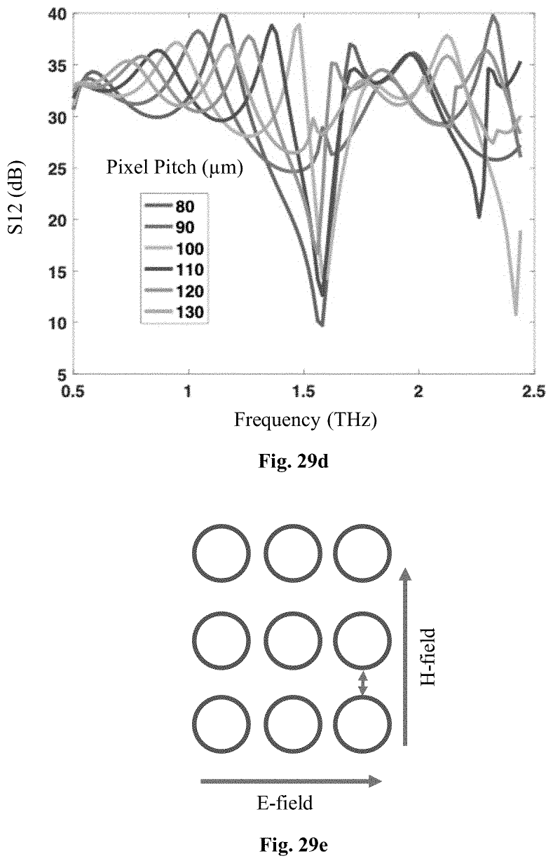

As an example, reference is now made to FIG. 29a showing the S21 spectra of the spiral nanoantenna structure in the container of the present embodiment for different pixel pitches that exhibit a spoof surface plasmon effect. As shown in FIG. 29b, this effect is a result of changing the spacing between metamolecules in the X-direction, which is the direction of the electric field that shifts the frequency of the SPP formed. This is actually the key characteristic of a surface plasmon wave. Further, FIG. 29c shows the spectrogram of the spiral nanoantenna structure in the container of the present embodiment for different pitches exhibiting spoof surface plasmon effect when the spacing between metamolecules is changed in the X-direction. This spectrogram of the SPP effect shows how the X-axis spacing between the contained spiral metamolecules actually opens and closes the SPP window which is due to radiative far-field surface coupling between the meta-molecules.

FIG. 29d shows the S21 spectra of the spiral nanoantenna structure in the container of the present embodiment for different pitches exhibiting spoof surface plasmon effect. As can be clearly seen in FIG. 29e, this effect is a result of changing the spacing between metamolecules in the Y-direction, which is the direction of the magnetic field. In this case, the spoof surface plasmon effect will collapse the SPP effect without shifting the frequency. This is also the key characteristic of a surface plasmon wave.

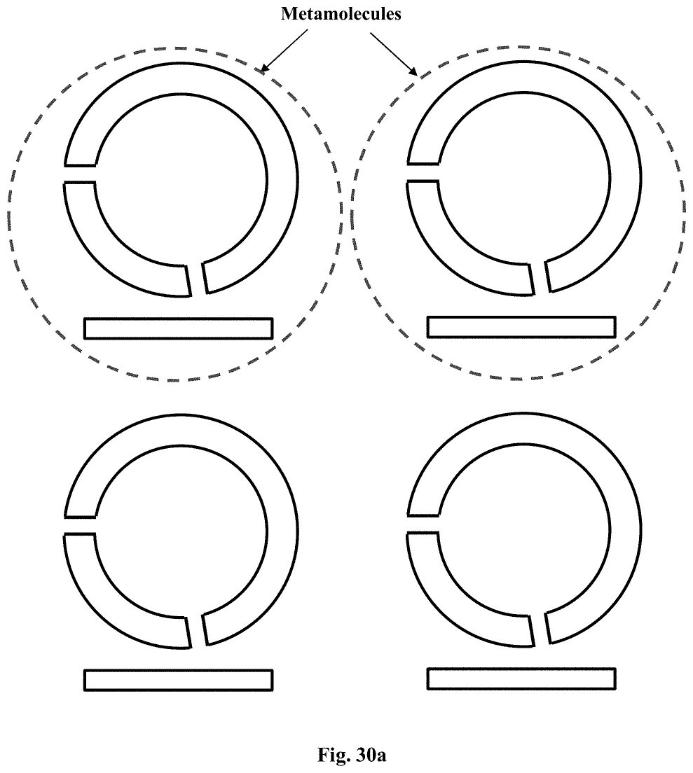

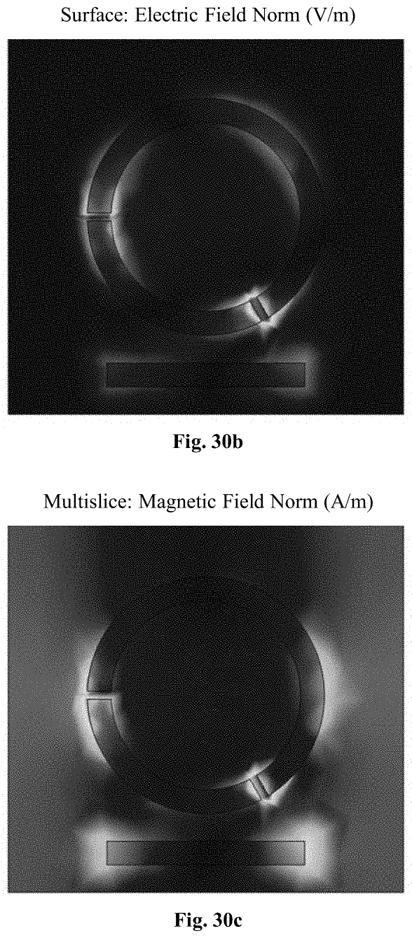

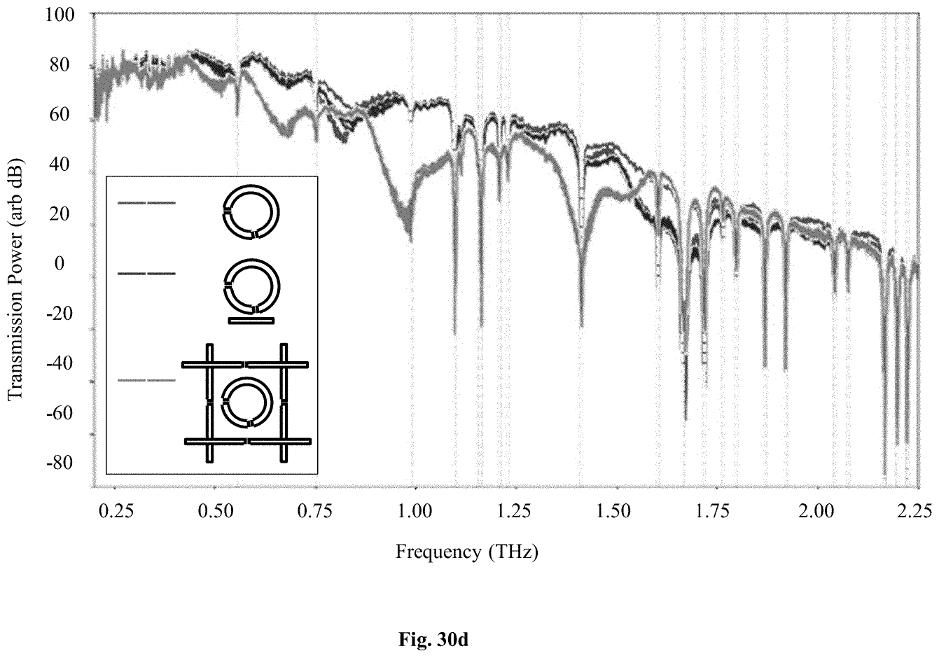

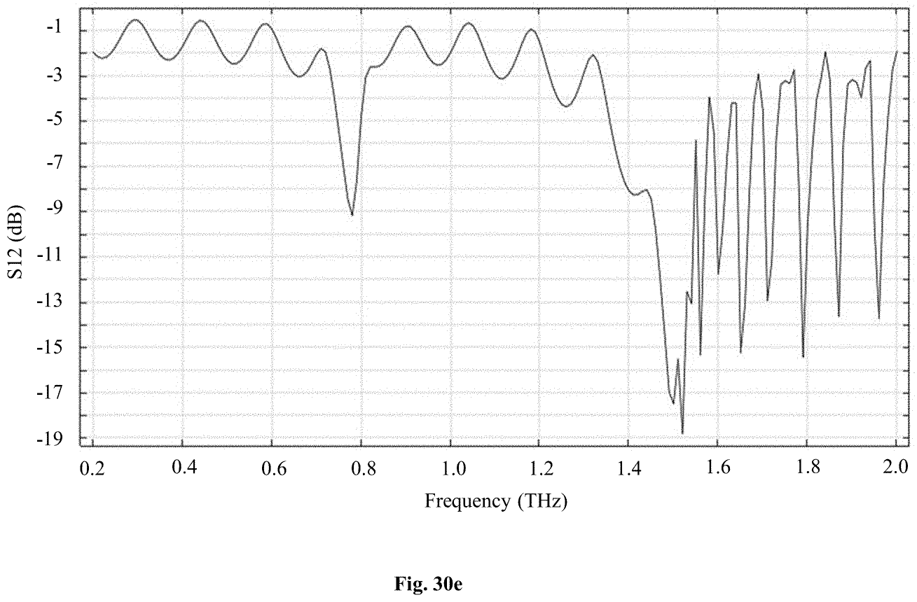

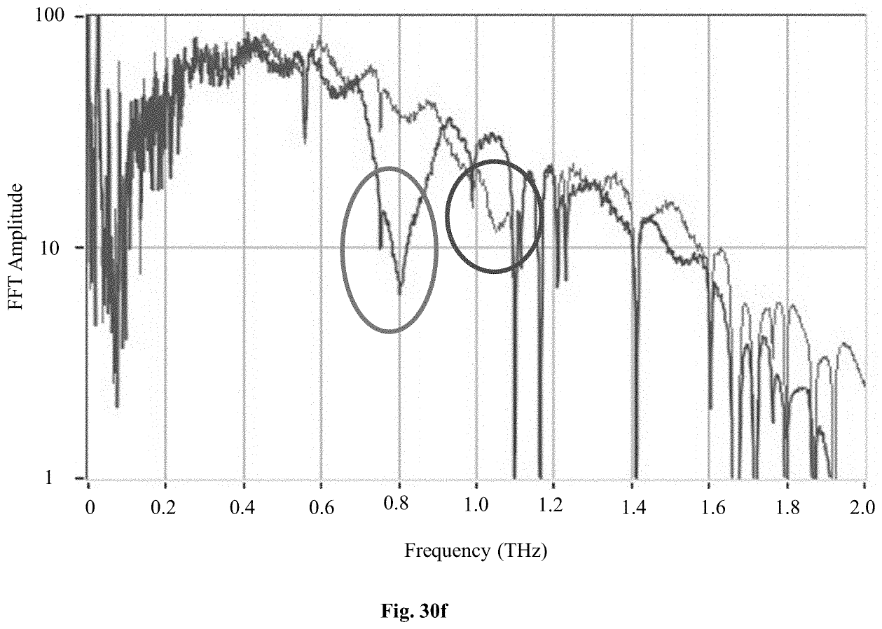

Reference is made to FIG. 30a schematically showing the nanoantenna array structure of the present invention, wherein each metamolecule in the nanoarray is composed of a round-shape split-ring resonator having two splits in the ring and a metal bar under the round-shape split-ring resonator. As above, the metal bar is a wave bouncer designed to bounce an incoming wave from the resonating split-ring and excite a dark mode followed by coupling it into the split-ring resonator. FIGS. 30b-30c show the electric and magnetic field distribution, respectively, inside another exemplary metamolecule of the present invention comprising an asymmetric split ring and a metal bar under this asymmetric split ring. FIG. 30b shows the electric field distribution (map) at the first frequency mode, which is known as the “fundamental mode”. FIG. 30c shows the magnetic field distribution (map) at that same frequency.

FIG. 30d shows the spectra comparison of three nanoarray structures containing a round-shape split-ring resonator with two ring splits. These nanoarray structures are different in having a wave container or bouncer. The first structure (green line) is a reference structure that does not have any wave container or bouncer. The second structure (violet line) has the metal bar as a wave bouncer under the split-ring resonator. The third structure (red line) has a metal square-shaped (box) as a wave container surrounding the split-ring resonator. Walls of this metal square-shaped wave container can be optionally symmetrically split to form additional resonance structures creating the bright mode. Since these splits are symmetrical, they can be used as a reference by rotating the chip. Resonance superposition and enhanced notch filter-like response is observed and can be enhanced by introducing sharp corners near the resonating gaps. FIG. 30e shows the THz multi-resonance spectrum and fingerprint region of this structure, which is used to demonstrate how the present invention allows to create the fingerprint “vibrational modes” of a specific compound, protein or virus.

There are two nano-gaps splitting the ring in the metamolecule shown in FIG. 30a. The asymmetry of these gaps in the ring creates metal regions of different lengths that produce cut-off bands in the spectra where transmission decreases sharply. In addition to the resonance from the oscillating field in the gaps in the ring, the current oscillation induced by the resulting magnetic field is responsible for the second resonance at 1.6 THz. More resonance bands in the sensor response provide greater sensing range in the THz frequency range.

FIG. 30f clearly demonstrates that using the structures of the present invention capable of creating the EIT window, it is possible to create a perfect wavelength comb which would hit many different specific vibrational modes of a tested compound, protein or virus. FIG. 30f is an excellent example of the wavelength comb that was tuned specifically for the SARS-CoV-2 virus at a lower frequency. It is truly incredible that the metamaterial of the present invention is capable of creating an EIT window and this material is all active at the same frequency. In other words, the THz spectra of this metamaterial does not show a bunch of lower order vibrational modes, but rather the same multiple frequency comb created by the interference that is occurring between the asymmetric ring and the bar underneath the ring. This phenomenon is defined in the present invention as “creating all the frequencies multiple resonances”.

The above described phenomenon of creating multiple resonances at all THz frequencies, which is discovered in the present invention is based on the method of vibrational mode matching for ultra-selective detection based on coupled modes between a sample and metamaterial. This method was also developed in the present invention. As mentioned above, it allows creating the fingerprints of “vibrational modes” of a specific compound, protein or virus, which are the frequencies that the compound, protein or virus is absorbing and emitting in the THz range and that match those of the specific e-field and h-field “vibrational modes” of the specific metamaterial of the present invention. Using the vibrational mode matching based on the coupled modes between a sample and the metamaterial of the present invention allows to significantly increase specificity of the sensor and assay of the present invention. Moreover, it allows to test complex and “dirty” samples and to match multiple vibrational modes to different resonance effects in the metamaterials of the present invention.

Furthermore, the multi-resonance metamaterials of the present invention allow creating many resonances, which can be specifically matched to the strongest vibrational modes of the compound, protein or virus (meaning the proteins and sugars which are the composition of the virus). Thus, the metamaterials of the present invention utilise both dark and bright modes to interact and create many different resonances, which are also defined as “trapped modes”. These trapped modes can be engineered to appear at the very specific frequencies of the vibrational modes of the target compounds, proteins or viruses. These vibrational modes then interact with the target compounds, proteins or viruses, and their interaction is based on the phenomenon of the vibrational mode matching of the coupled modes between the sample and metamaterial as described above. The uniqueness of this technique developed in the present invention is that when there is a matching between the vibrational modes and one of the resonances, which is created by the interference of the dark and bright modes (the container and the asymmetric split-ring structure), a unique pattern of interference is actually created.

In support of the above conclusions, reference is now made to FIG. 30f demonstrating the difference between the square split-ring resonator structure described in the prior art, for example Ji-Hun Kang et al. (2018), and the nanoantenna structure of the present invention, which utilises the effects of radiative coupling to boost the Q-factor and resonance in the THz range. The blue line stands for the sensor of the present invention, inducing the dark mode in a split-ring resonator, with a blue circle showing the resonance peak, while the red line is for the prior-art sensor operating mostly in the bright mode with a red circle showing its resonance peak.

### Fractals as an Embedded Lattice

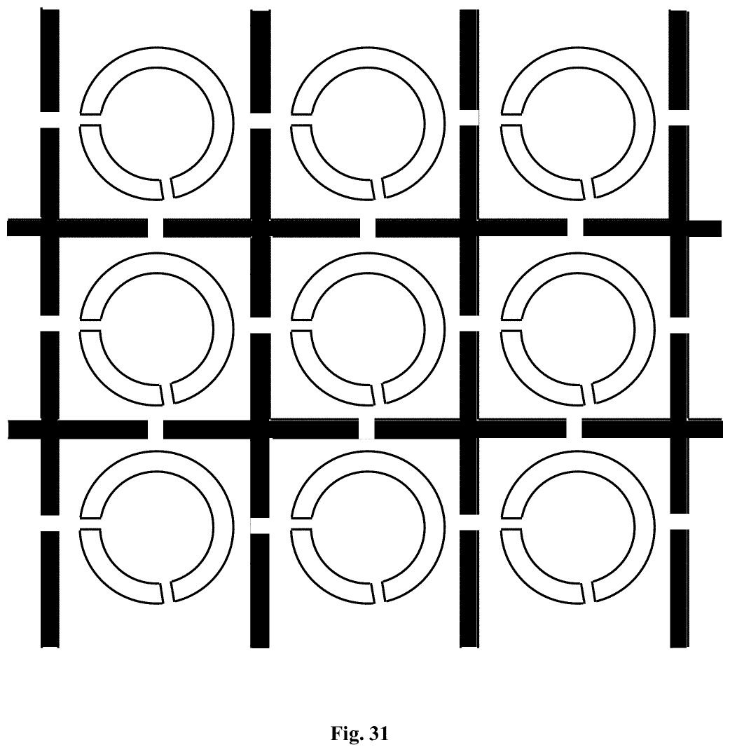

The space filling ability of fractals is used in the present invention to scale up the sensing area without increasing the overall dimensions of the sensor chip, while increasing the radiation efficiency of the nanoantenna owing to several intricate corners and features within the structure geometry. One of the fractals, mentioned above (see FIG. 30d), and shown in FIG. 31 comprises an array of round (circular)-shape split-ring resonators having at least two splits in the ring and a metal square-shaped container (box), said metal square-shaped container is designed to excite a dark mode in the round-shape split-ring resonators, followed by coupling it into said split-ring resonators. Thus, in a specific embodiment, each metamolecule of the nanoantenna structure is composed of a round (circular)-shape split-ring resonator having at least two splits in the ring and a metal square-shaped container (box), said metal square-shaped container is designed to excite a dark mode in the round-shape split-ring resonator, followed by coupling it into said split-ring resonator. Walls of this metal square-shaped wave container can be optionally symmetrically split to form additional resonance structures creating the bright mode. Since these splits are symmetrical, they can be used as a reference by rotating the chip.

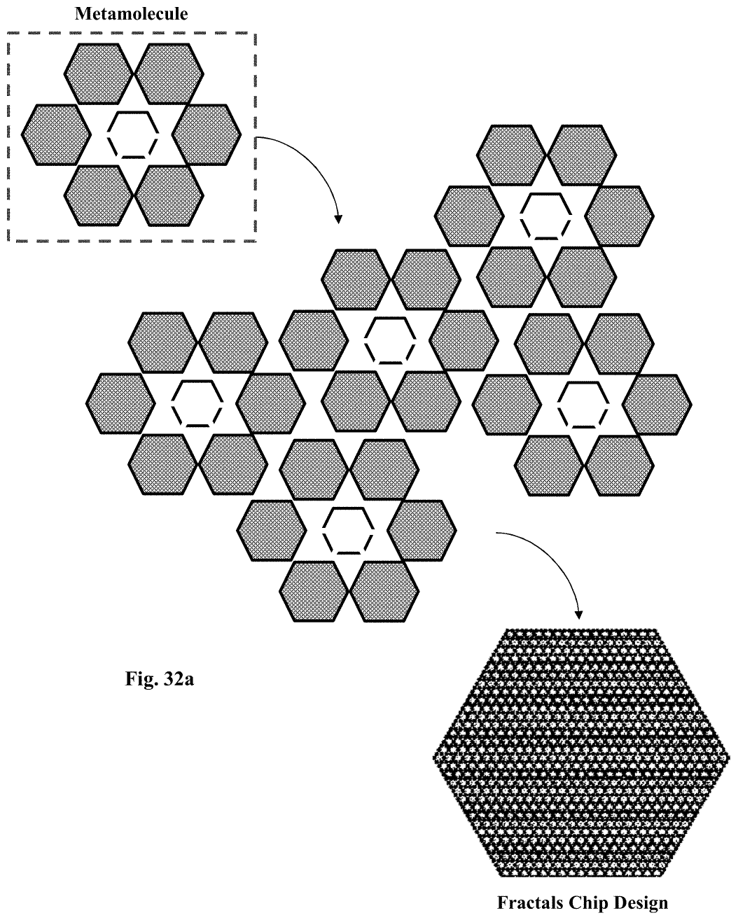

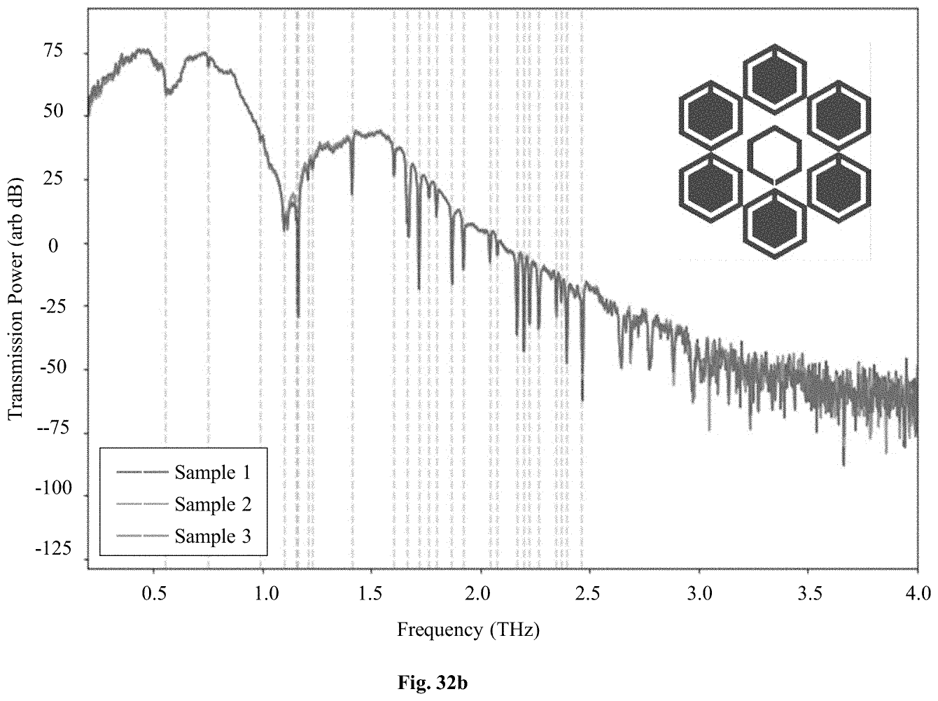

Reference is now made to FIG. 32a showing the nanoantenna array and a single metamolecule building this array. In this specific embodiment, each metamolecule is composed of a hexagon-shape split-ring resonator having at least one split in the ring and six metal hexagons surrounding said hexagon-shape split-ring resonator, said metal hexagons are designed to excite a dark mode in the (inner) hexagon-shape split-ring resonator, followed by coupling it into said hexagon-shape split-ring resonator. Hexagonal symmetry is employed in the present embodiment of the nanoantenna structure of the invention because it has the closest packing and highest rotational symmetry. This increases the total perimeter of the metal patch and can be used to manipulate the magnetic resonance in the asymmetric split-ring resonators embedded together with the hexagonal plates and without increasing the form factor of the sensor. Further nesting the complementary nanoarray structure as shown in FIG. 32b into the outer plate results in a dramatic increase in the resonance Q-factor. FIG. 32b demonstrates the corresponding FFT THz spectra of this nested design.

### Index-Matching Layers (PMMA) to Reduce Internal Reflections

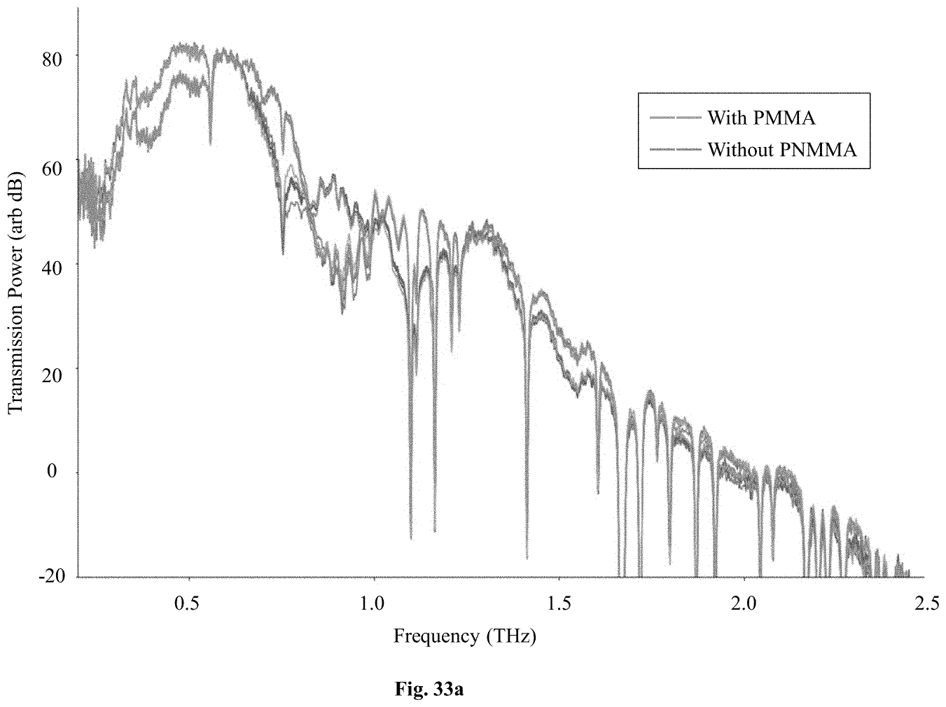

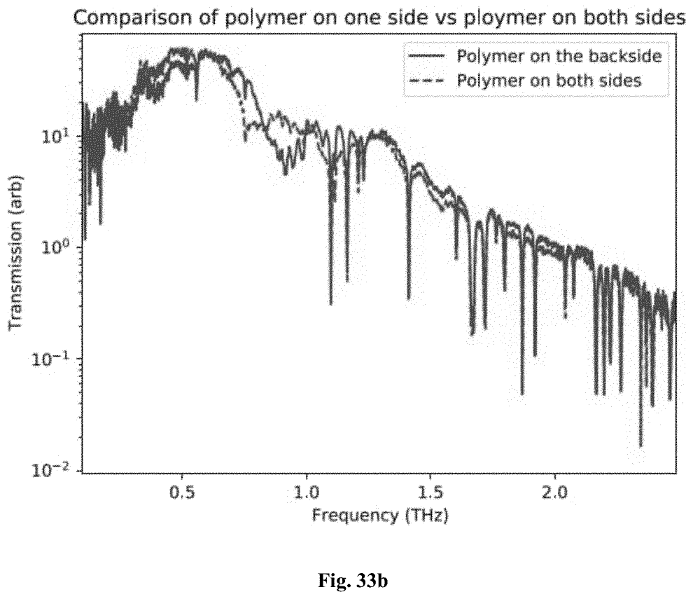

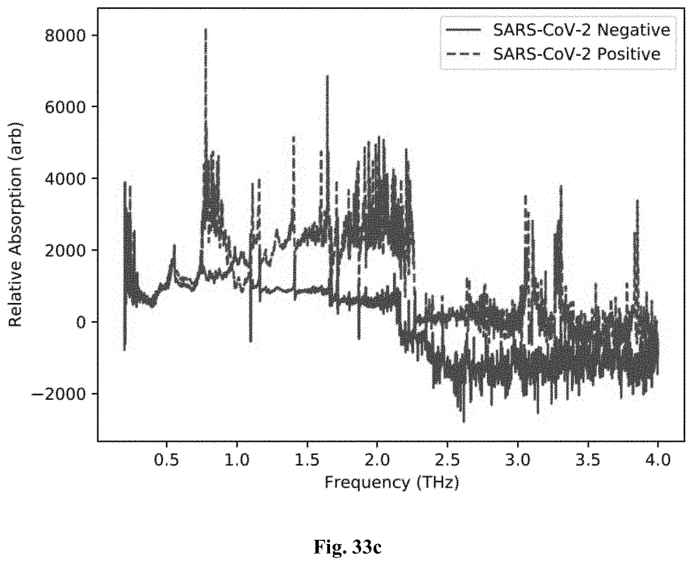

Applying index matching foils on both sides of the sensor e.g. PMMA considerably reduces internal reflections, resulting in a smoother spectrum. The material parameters of the foil can be used to tune the position of the sensor resonances as shown in FIG. 33a, in the experiment using 50 μm PMMA foils. As seen in FIG. 33a, a significant redshift of about 200 GHz is clearly observed in the reference sensors with the PMMA layers (pink spectra lines) compared to bare sensors without any PMMA layers (light blue spectra lines). Application of PMMA increases the sensor's filter characteristics resulting in steeper roll-off regions (e.g. at 1.05 THz) which is very useful in sensing applications.

Reference is made to FIG. 33b comparing the sensor response using the hexagonal fractal-design sensor of the present embodiment with the embedded hexagonal asymmetric split-ring resonators for the sensor chip with PMMA on the backside of the chip (green curve) versus the same sensor chip with PMMA on both sides of the chip (red curve). FIG. 33c showing the rapid detection, tested on positive and negative samples, with the PMMA layers deposited on the surface of the sensor. SARS-CoV-2 positive samples (red curve) and negative control samples (green curve) were applied over the PMMA. As seen in FIG. 33b, the generated spectra show distinct features in their sample absorption (coefficient) spectra which is a basis for efficient classification of the swab samples at the point of care.

The use of the PMMA foils with an adhesive layer (for example, PET-based) is suitable for samples transfer from swabs collected from patients. More uniform sample coverage and transfer is achieved using these foils as compared to using bare sensors, e.g. metamaterials on quartz. A foldable booklet/centre page-like scheme is used to bring the sample (left side of the booklet) in close contact with the metamaterial (right side of the booklet), as well as to minimise thickness variations in the sample due to the stacking on top of the present sensor (which is PMMA plus adhesive), in the middle (which is the patient's sample), and at the bottom layers (which is the metamaterials stack with an index matching layer).

### Devices and Applications of the Present Invention

One of the essential aspects of the present invention is that in contrast to other analytical techniques and devices, the microelectronic sensor of the present invention is capable of analysing “dirty” samples without purification, separation and washing. In some embodiments, the sample collection system is a sampling swab attached to the microelectronic chip. The sample taken with the swab contains a mixture of various organic and inorganic compounds and biological species. Such sample is directly transferred to the nanoantenna without any pre-treatment.

In further embodiments, the sample collection system is a breathalyser incorporating the microelectronic sensor of the present invention. The sample is then an exhalation air blown by an end user into the breathalyser. It contains a mixture of various organic and inorganic compounds and biological species. The exhalation air containing droplets of saliva is filtered before entering the nanoantenna with at least one suitable filter, details of which will be provided below.

The present application describes embodiments of a breathalyser for label-free chemical detection and biomolecular diagnostics of a “dirty” sample, which is a raw breath sample received directly from a subject being tested without any pre-treatment or purification and without any chemical or biological separation. The breathalyser of the invention comprises:

an integrated exhalation portion placed in a housing, said housing is transparent to terahertz (THz) radiation and designed to collect a sample of exhalation air and transfer said sample to a testing chamber; and

the testing chamber integrated inside said housing, attached to said exhalation portion and designed to receive, filter and analyse said sample, wherein said testing chamber comprises:
(a) at least one filter suitable for filtering the sample;
(b) a microelectronic chip comprising a nanoantenna structure, wherein said nanoantenna structure is arranged in a periodic array of metamolecules and configured to detect and transmit signals through said testing chamber in a terahertz (THz) frequency range; and
(c) an integrated circuit suitable for storing and processing signals in a THz frequency range, and for modulating and demodulating radio-frequency (RF) signals;
characterised in that each of said metamolecules in the array is composed of at least one split-ring resonator and a wave container or a wave bouncer, said wave container confines and said wave bouncer bounces electromagnetic waves received from said at least one split-ring resonator, both the wave container and the wave bouncer are designed to excite a dark mode in said at least one split-ring resonator, followed by coupling the excited dark mode back into said at least one split-ring resonator.

A breathalyser is a device used for qualitative and quantitative chemical detection and biomolecular diagnostics from a breath sample. The name Breathalyzer is a genericised trademark of the brand name of instruments developed by Robert Frank Borkenstein in the 1950s. Today, these devices are used in various chemical and biomolecular applications. There are many different types of breathalyser sensors available in the market, for example photovoltaic breathalysers, infrared breathalysers, fuel cell gas sensors, semiconductor breathalysers.

The exhalation portion of the breathalyser of the embodiments serves for blowing air (breath) containing an analyte to be tested onto the microelectronic chip installed inside the testing chamber. The breathalyser further contains a disposable adapter attached to the exhalation portion and suitable for receiving the exhalation air and transferring it to the exhalation portion of the breathalyser. The collected sample of the blown (exhalation) air flows through the exhalation portion to the testing chamber, is filtered there by passing through at least one suitable air filter, followed by projecting on the microelectronic chip of the present invention. This way, the aerosol droplets and water vapours carrying bio-molecules and viruses, as well as other airborne particles in the exhaled breath can be collected as micro-droplets on the sensing nanoantenna structure.

The exhalation portion, testing chamber and their dimensions are fully customizable and determine the amount and particle size of the aerosol particles collected from. The testing chamber is designed in such a way that it allows the droplets to dry in a very short period of time, less than 60 seconds. The breathalyser of the present embodiments is completely transparent to THz radiation.

The exhalation portion of the breathalyser optionally has a soft membrane or mechanical valve which is designed to move based on the air pressure to ensure that there is a sufficient amount of the blown air collected for measurements (a user has blown hard enough into the breathalyser and the proper amount of breath particles and vapours have been collected). The breathalyser has optionally a teeth grip ring for easy use and firm holding, so that when blowing, it does not pop out of the user's mount. The breathalyser of the present invention is a single-use device and can be operated entirely by the user with no help from medical personnel. Optionally, it can be integrated into a medical setting and used by medical personnel.

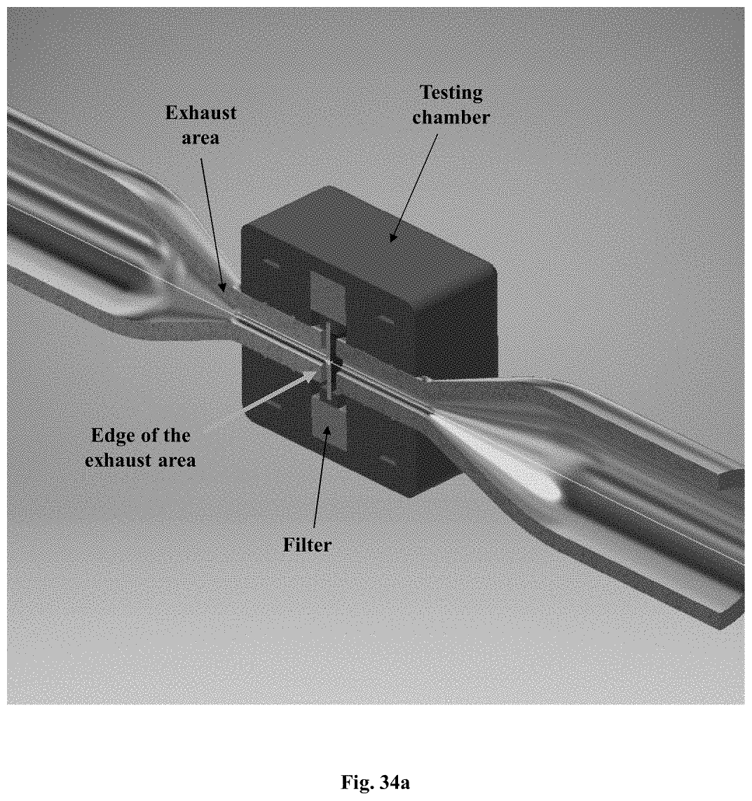

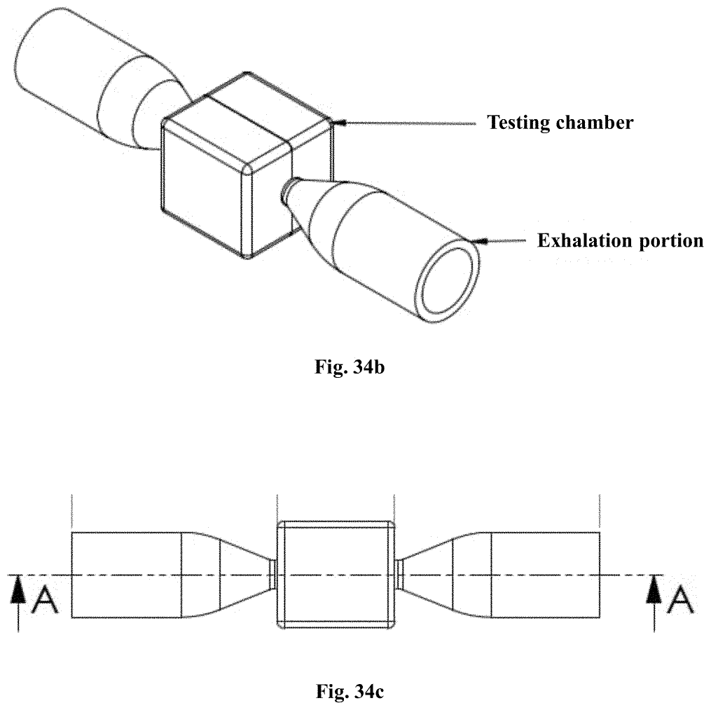

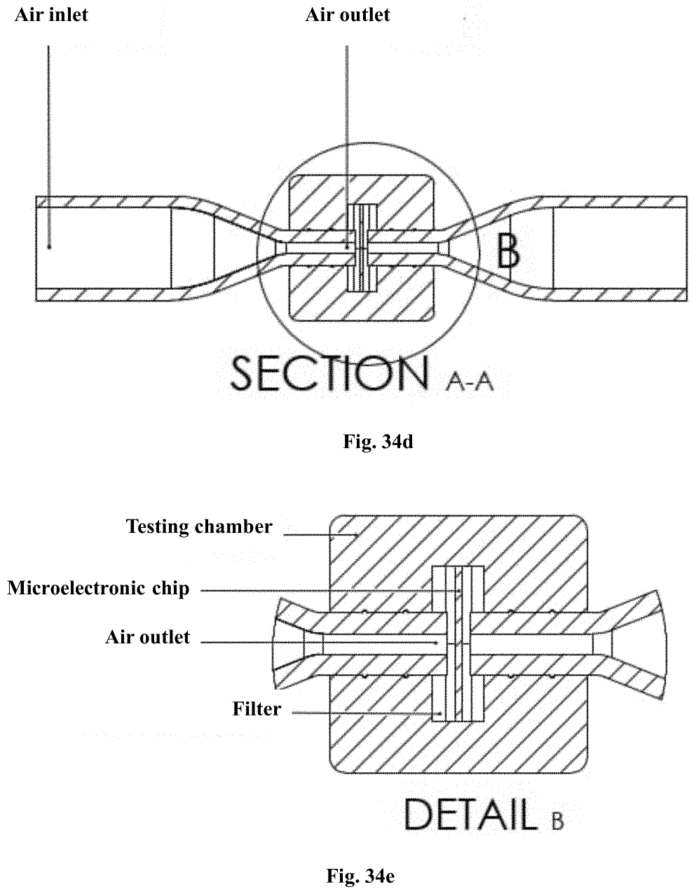

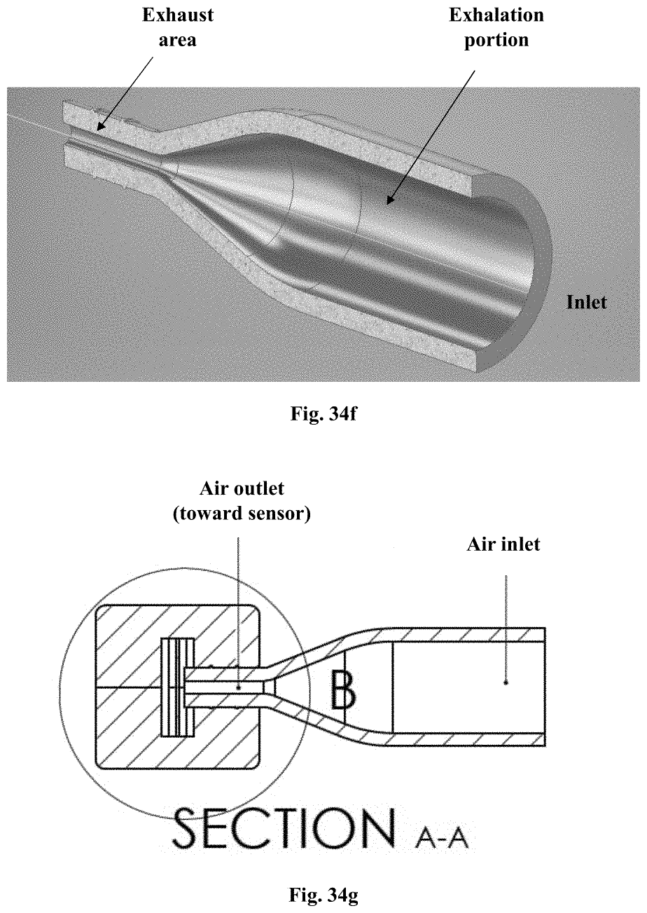

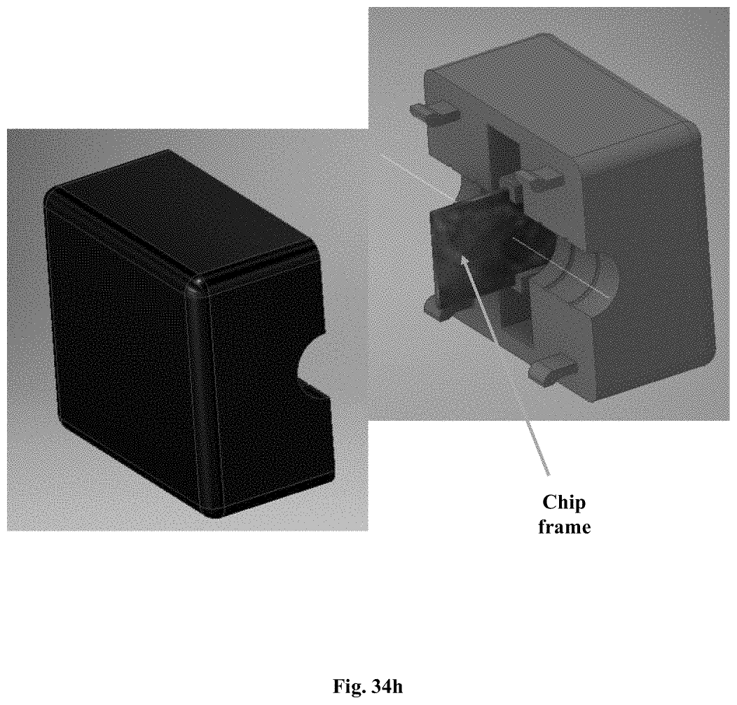

The testing chamber of the present invention contains the microelectronic chip of the present invention for detecting analytes in the sample and has a unique structure adapted to fluid dynamics of the sample. Reference is now made to FIGS. 34a-34e showing the testing chamber integrated into the housing and attached to the exhalation portion of the breathalyser. As can be seen from these figures, the exhaust area of the exhalation portion is significantly reduced, thereby significantly increasing the Stokes number characterising the behaviour of particles suspended in a fluid flow of the sample. The Stokes number is defined as the ratio of the characteristic time of a particle (or droplet) to a characteristic time of the flow or of an obstacle, where the obstacle in the present case is the microelectronic chip installed inside the testing chamber to receive the particles or droplets from the exhalation air.

FIGS. 34f-34g show the exhalation portion of the breathalyser of the present invention. Exemplary dimensions of the exhalation portion in a form of a tube are as follows: 55 mm length, 14 mm inlet diameter and 2 mm exhaust area diameter. The proposed design of the breathalyser exploits the Venturi effect to accelerate the flow of the sample before hitting the microelectronic chip surface. In general, the Venturi effect is the reduction in fluid pressure that results when a fluid flows through a constricted section of a pipe, which is the exhaust area of the exhalation portion in the present design. The exhaust area is elongated in order to bring the exhalation portion closer to the microelectronic chip. Based on the above exemplary dimensions, the ratio between the inlet area diameter and exhaust area diameter is 12.25. The Venturi effect and the calculations leading to the proposed design will be discussed below.

FIG. 34h shows the testing chamber of the breathalyser of the present invention. As can be seen from the figure, the testing chamber consists of two interconnecting bodies. The frame for positioning of the microelectronic chip is inserted in the centre of the testing chamber. The testing chamber also provides housing for the integrated circuit, battery and any other electronic components. As shown further in FIG. 34i, the chamber also comprises at least one filter suitable for filtering the sample. Non-limiting examples of the suitable filters are high efficiency particulate air (HEPA) filters and ultra-low particulate air (ULPA) filters designed to trap a vast majority of small particulate contaminants from an air stream. According to the US standard DOE-STD-3020-2005, a HEPA filter must be capable of removing 99.97% of contaminant particles of 0.3 m in diameter. It also specifies that HEPA filters must feature minimal pressure drop and maximum airflow when in operation. Ultra-low particulate (or sometimes “penetration”) air (ULPA) filters are closely related to HEPA filters but are even more efficient. ULPA filters are specified to remove 99.999% of contaminants of 0.12 m or larger in diameter. In a certain embodiment, the testing chamber further comprises clamping fixtures between the chamber parts.

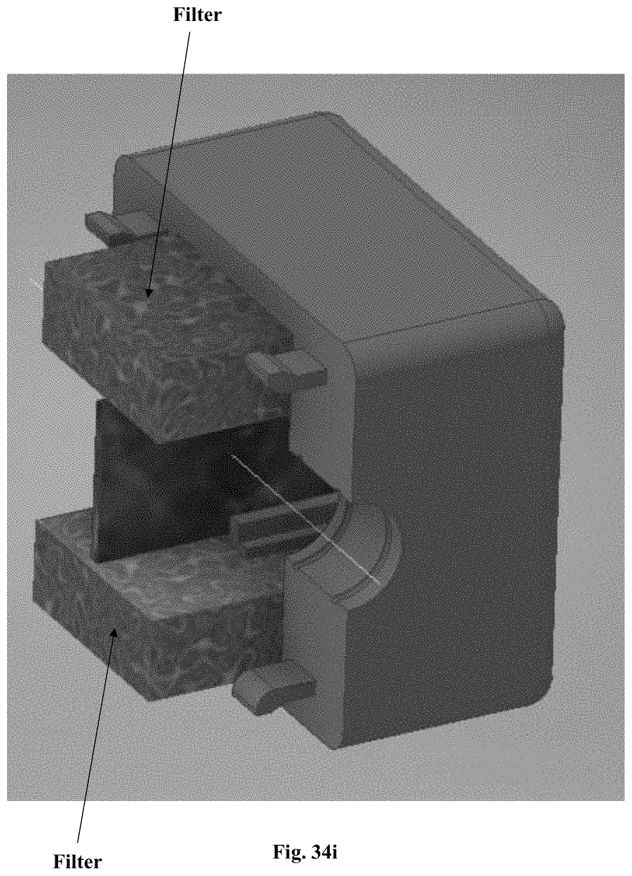

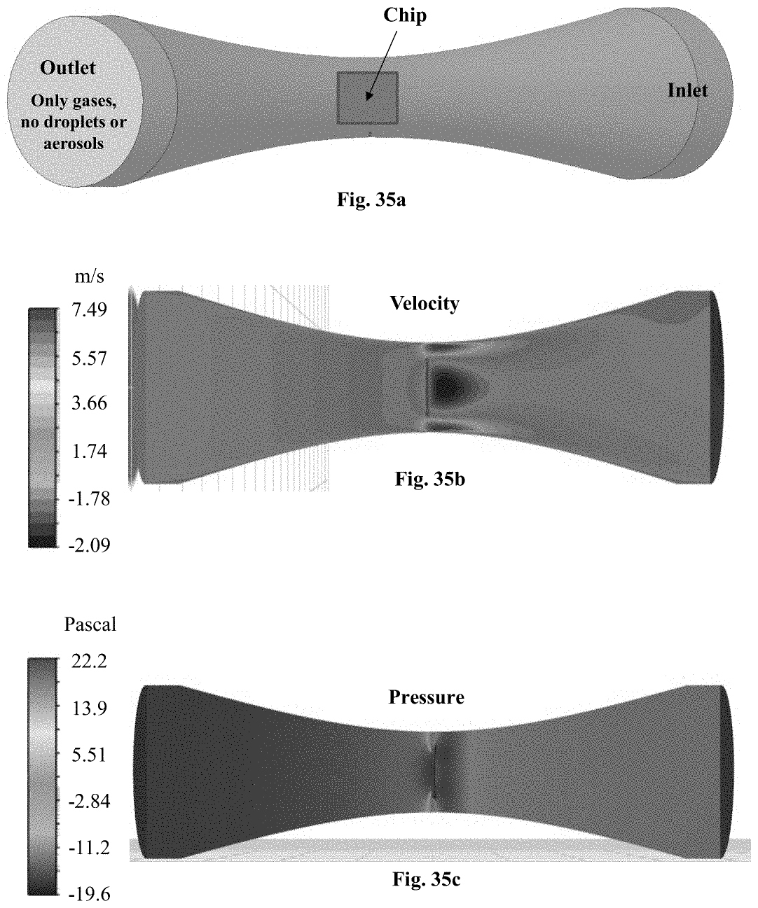

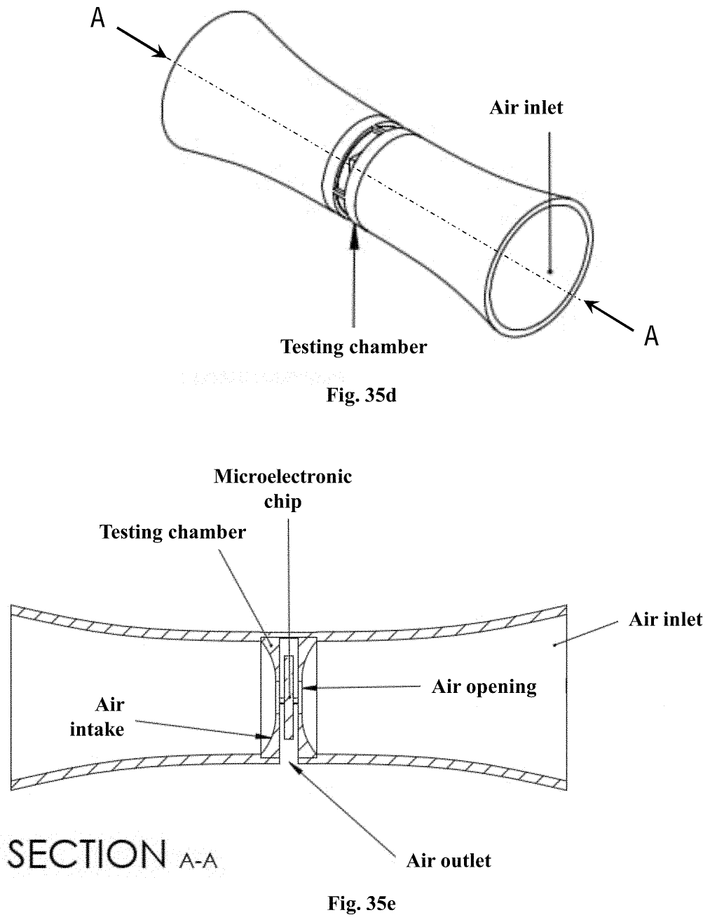

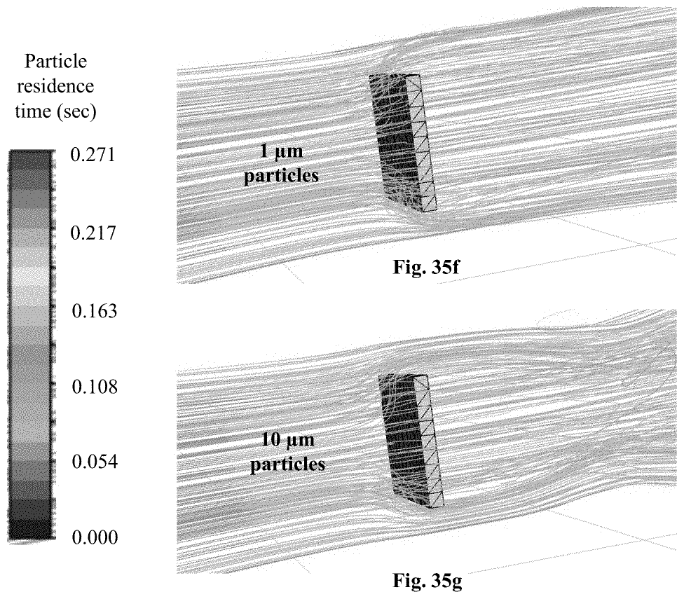

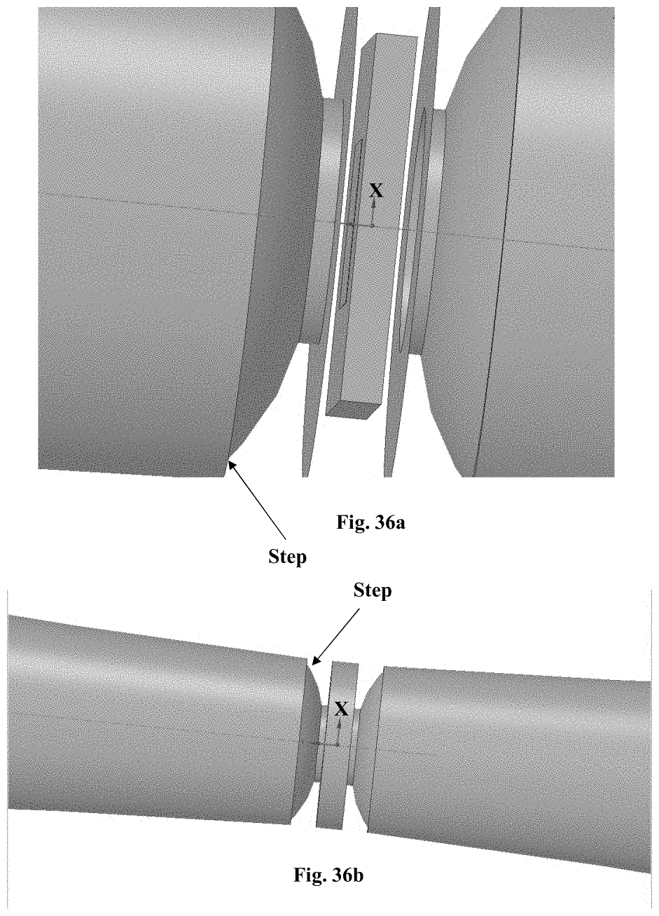

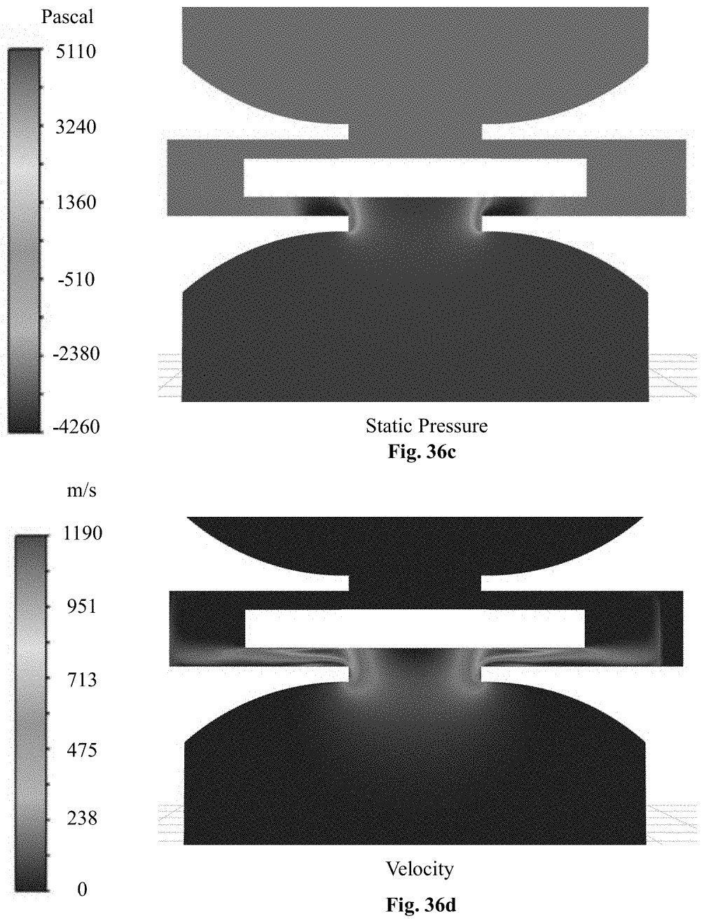

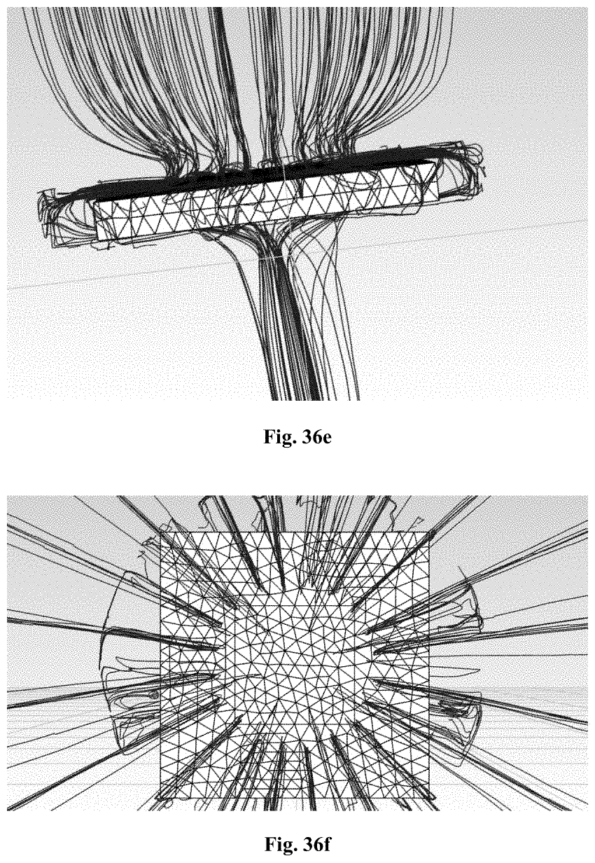

Reference is now made to FIG. 35a showing the initial design of the breathalyser of the present invention, which is tubular and known as a Venturi tube. Velocity and pressure simulations for the initial design using mass-flow inlet condition of 0.4 L/s of air are shown in FIGS. 35b and 35c, respectively. FIGS. 35d and 35e show the similar Venturi tube design of the breathalyser of the present invention. FIGS. 35f and 35g show initial simulation results for particles in the air guided around the chip, with 1 μm particles not hitting the chip surface and 10 μm particles producing the 2.5% hit rate on the chip surface, respectively. This motivated using a focusing element, which is a part of the design shown in FIGS. 36a and 36b. The exemplary focusing element has 6-mm diameter with 0.5 mm gaps on each side of the chip. As seen in these figures, there is a small step in the wall that causes particles to recirculate.

The velocity and pressure simulation for the computational fluid dynamics model (CFD) of this design are shown in FIGS. 36c and 36d, respectively. FIGS. 36e-36f show the results of the computer simulation for this design. The particles are now being focused and hitting the chip but outside the nanostructure area. To overcome this problem, the chip was moved closer and the focus diameter was increased.

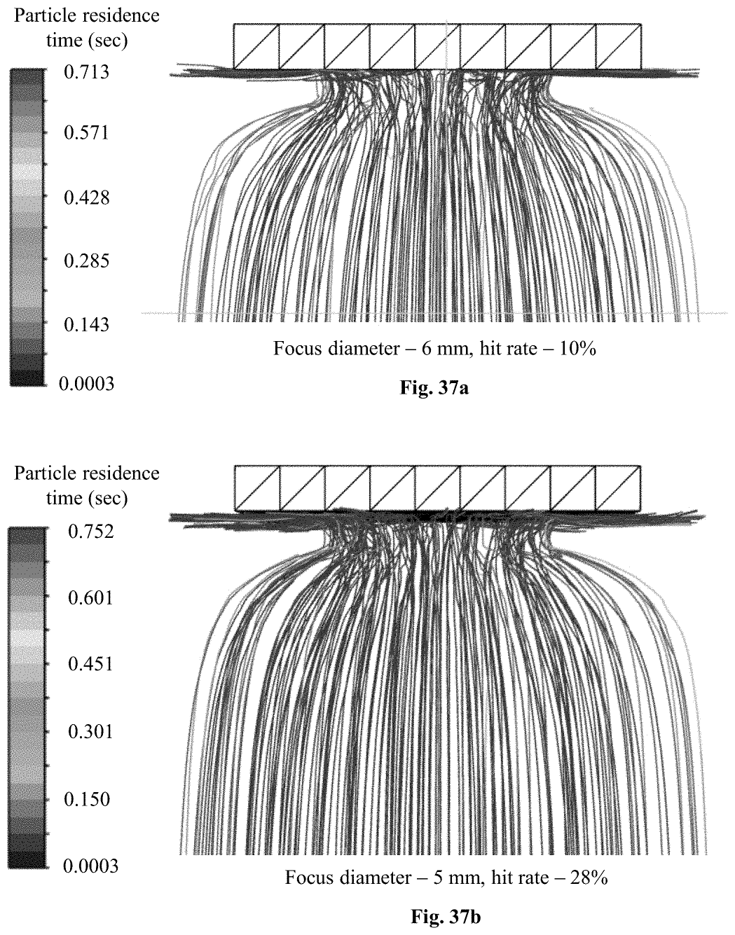

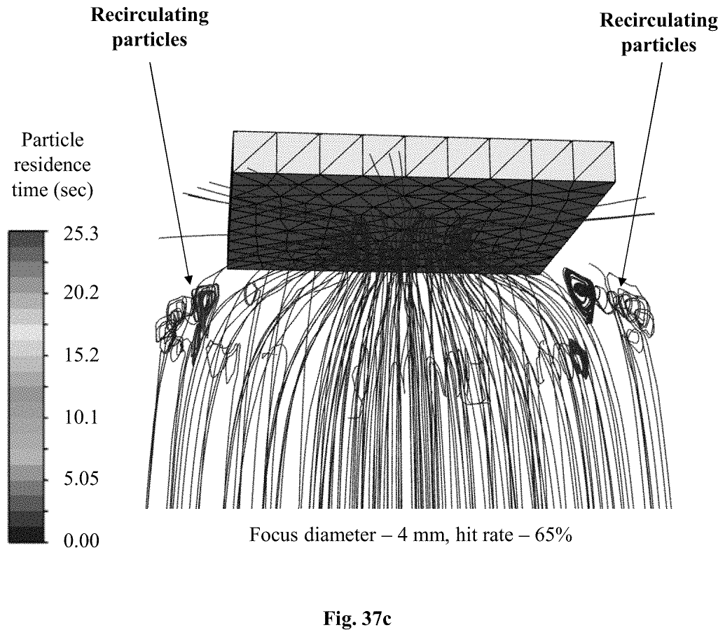

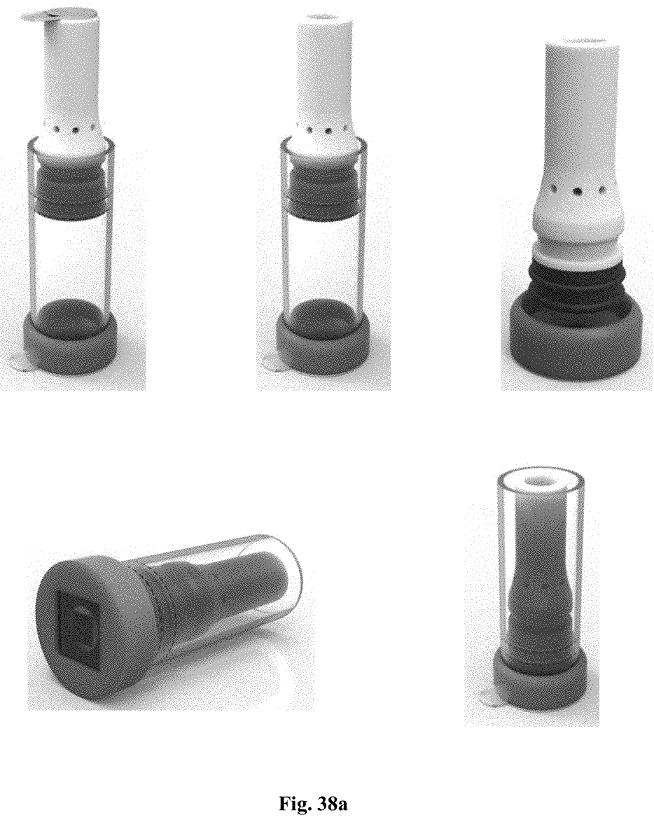

Reference is now made to FIGS. 37a-37c showing the simulation results for open centre designs. As seen in these figures, change in the outer radial boundary of the central cylinder from a wall to an outlet boundary and the focus diameter significantly affect the hit rate. Decreasing the focus diameter from 6 mm (FIG. 37a) to 5 mm (FIG. 37b) and then to 4 nm (FIG. 37c) increases the hit rate from 10% to 28% and to 65%, respectively. Further increasing the diameter to 3 nm and thus increasing the air pressure on the chip surface offsets the focusing and the hit rate plateaus at 65%. As seen in FIG. 37c, at higher air pressure, there are particles recirculating at the boundaries.

The above proposed design proved the mode of operation but did not result in flow speeds and Stokes numbers adequate for the impact of pathogen or virus particles on the chip surface. Since these particles are very small, the flow must be accelerated much. This is because in fluid dynamics, an incompressible fluid's velocity must increase as it passes through a constriction in accordance with principle of mass continuity, while its static pressure must decrease in accordance with Bernoulli's principle of conservation of mechanical energy. Thus, any gain in kinetic energy a fluid may attain by its increased velocity through a constriction is balanced by a drop in pressure.

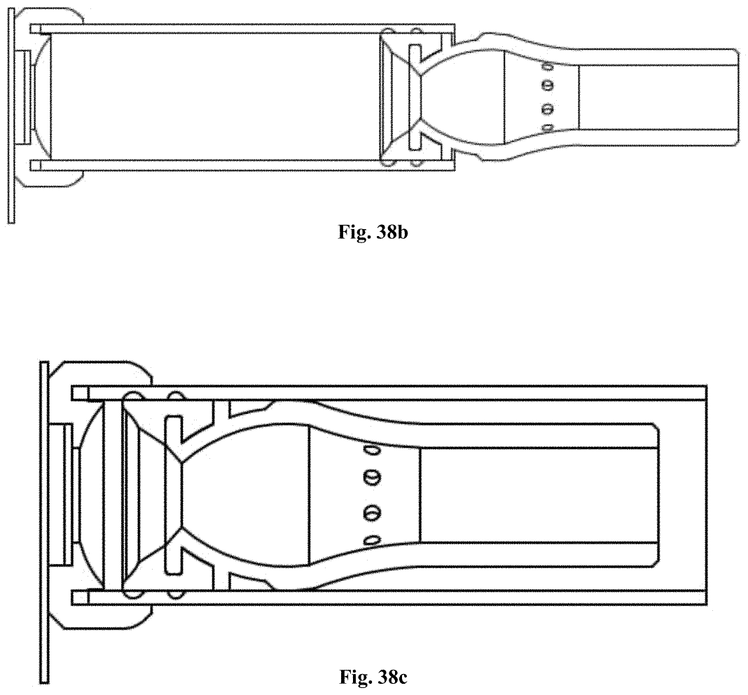

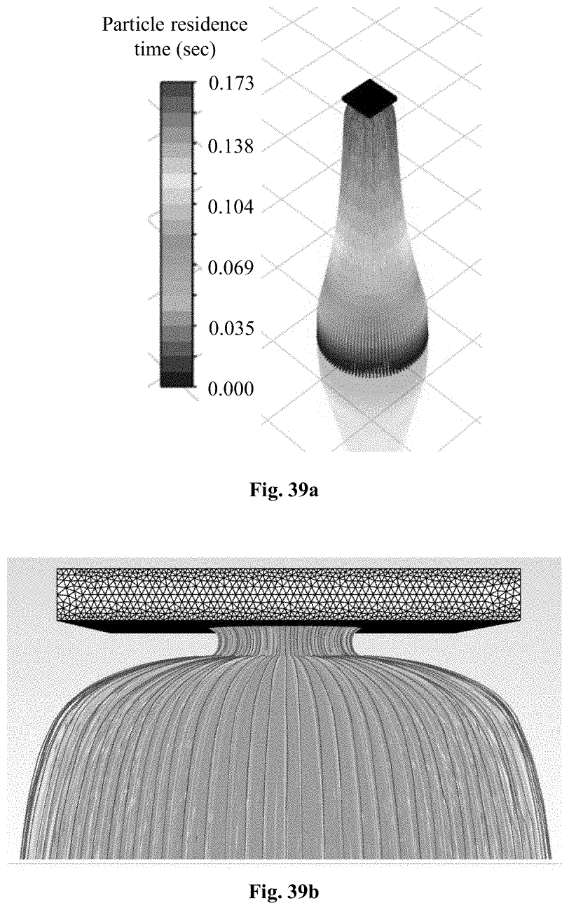

The above simulations and considerations brought the inventors to the breathalyser design of the “non-Venturi” type. FIG. 38a shows the marketing example of the breathalyser of the present invention, and FIGS. 38b-38c show the design of this breathalyser. FIG. 39a show the simulation model of this breathalyser, having 3.5 mm diameter focus and smaller spacing between the focus and the chip resulting in the 100% hit rate. FIG. 39b shows particle traces coloured by particles residence time hitting the chip surface. Being essentially advantageous over other designs, this breathalyser still has some downsides. It is too long for optics system; the mouthpiece is too large; and too much pressure (about 2.5 psi) is required to blow into the exhalation portion (tube).

To improve this non-Venturi design, the following adjustments have been currently made. Spacing around the chip was increased to 1 mm on each side, thereby lowering the pressure needed to blow air into the exhalation portion of the breathalyser, but unfortunately decreasing the hit rate to less than 30%. With 0.75 mm spacing on each side of the chip, the hit rate was increased to 45%. Further minimising the spacing to 0.5 mm on each side of the chip drove the hit rate back up to 100%. This spacing of 0.5 mm requires only 5 kPa of the exhalation air pressure, and velocity of the escaping air is below compressible regime. To fit the optics system into the device, the tube length of the exhalation portion (without a mouthpiece) was scaled down to 60 mm. The mouthpiece was scaled down to 20 mm length for easier fit and air blow.

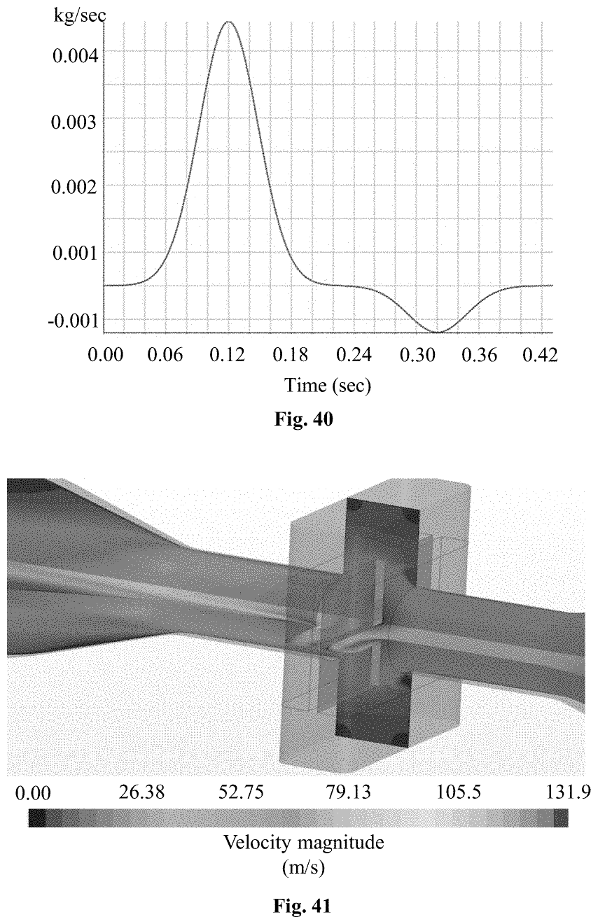

To overcome the above drawbacks of the non-Venturi design, the Venturi design was selected as an alternative. For the initial Venturi design, the exhalation air from breath has been modelled as a Gaussian pulse. Reference is now made to FIG. 40 showing the Gaussian pulse, which is shaped as a Gaussian function and has the properties of maximum steepness of transition with no overshoot and minimum group delay. It determines the flow rate at the air blowing side (inlet) of the exhalation portion. The air blown into the exhalation portion is assumed to be an ideal gas having no particles present in the initial design. Therefore, the exhalation air is modelled as air following the ideal gas law, in order to significantly reduce the computation time. A tested analyte, for example a pathogen or virus, was modelled as small spheres of 0.1 m diameter having density of 1185 kg/m3 which is a density of Influenza A virus. Drag and lift taken from the air flow were acting on the particles. Their distribution in time at the inlet of the exhalation portion followed that of the breath pulse with a small lag. These particles were bouncing when they hit the chip surface, which happens very quickly. Therefore, it was necessary to use small step sizes in the simulation.

Reference is now made to FIG. 41 showing results of the simulation for velocity of the exhaled air flow inside the Venturi tube contouring at two normal midplanes of the testing chamber. It is clearly seen in this figure that the air flow through the filters occurs above and below the chip. Also, the air flow velocity at the surface of the chip has reached more than 40 m/s at 85% of the time before registering of the breathing pulse. This impact rate is not sufficient to ensure the impact of the particles onto the chip, and the Stokes number is relatively low. Velocity magnitude showed in this figure indicates the flow of the fluid particles hitting the chip surface and passing through the filters.

In the Venturi design shown above, due to the Venturi effect, the air flow is much accelerated and rapidly reaches high velocity at the chip surface. This is beneficial for the mode of operation, when the particles are hitting the chip surface. However, the drawback of this design is that the air flow is not accelerated much through in the exhalation portion distanced from the chip, but only in the exhaust area. The edge of the exhaust area is not close to the chip which results in particles escaping from hitting the chip surface due to their low inertia.

As mentioned above, in the current Venturi design of the present invention, which is shown in FIG. 41, the exhaust area of the exhalation portion is significantly reduced, thereby significantly increasing the Stokes number characterising the behaviour of particles suspended in a fluid flow of the sample. The edge of the exhaust area of this breathalyser is very close to the chip surface. For the purpose of the computer simulation, the ratio of the inlet to the exhaust area was taken as a reference. The obtained Stokes number is 0.02635 for the proposed Venturi design. Virus particles are presented in this simulation model.

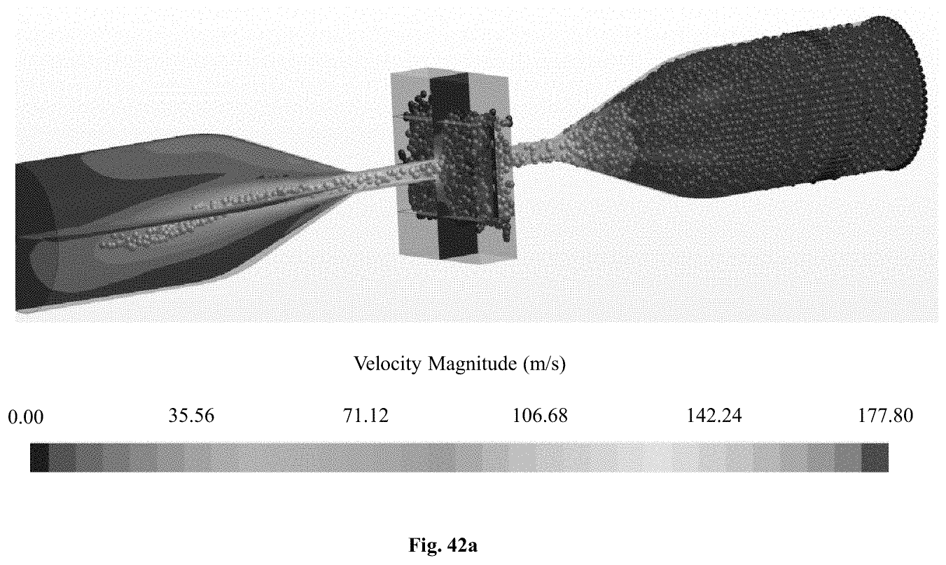

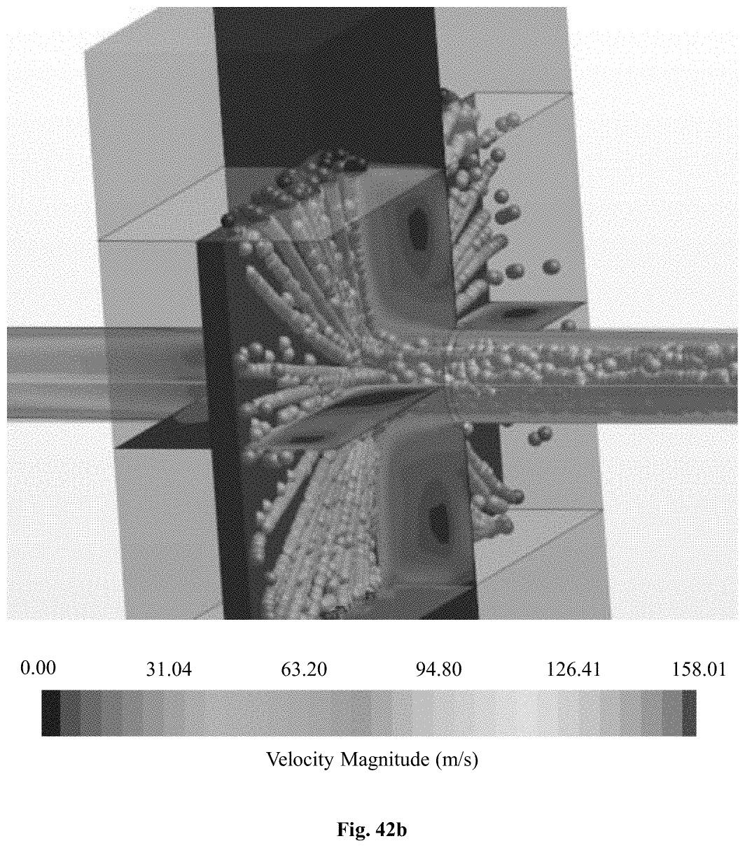

Reference is now made to FIG. 42a showing the simulation of the Venturi design of the breathalyser of the present invention. FIG. 42b shows the expanded view of the exhaust area. As seen in the figure, the first moments of the particles impact on the chip surface were monitored. This is possible because of narrowing of the exhaust area and because of the edge of the exhaust area being close to the chip. Particles pass through the filters and hit the chip surface. The hit (or impact) rate at initial phase of the impact (at 0.113852 sec of the simulation time) that is before the peak of the breath manoeuvre, is estimated to be 282 particles/m² on the chip surface. The hit (impact) rate at 0.113982 sec of the simulation time is 604 particles/m² on the chip surface.

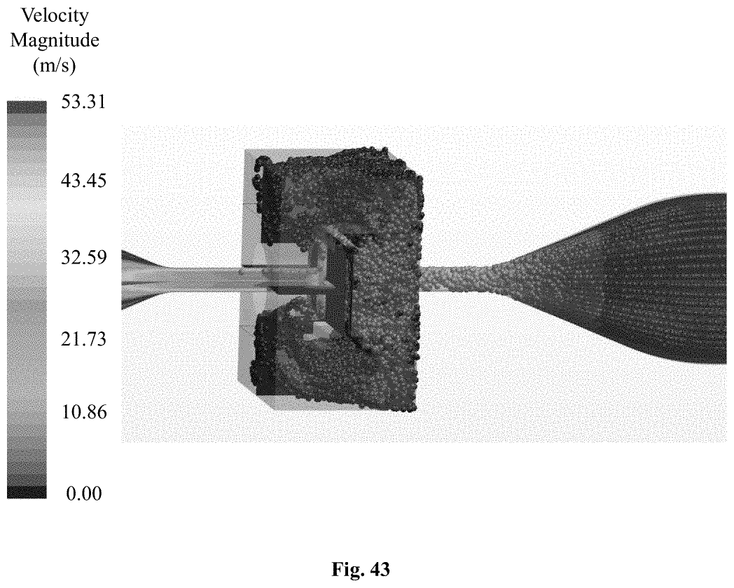

FIG. 43 shows the simulation results for the breathalyser of the present invention using the parameters of the SARS-CoV-2 virus. Virus is modelled as spheres of 0.1 m in diameter having constant particle size and density of 1185 kg/m³. No interaction between the virus particles is assumed. Number of virus particles used in the computational fluid dynamics (CFD) model is 1000. Particles are injected in the tester 0.05 sec after the initial breath pulse until its peak (0.15 sec). Breath is modelled as a Gaussian pulse with low breath intensity to see how the breathalyser is behaving at a low volume breath. Maximum flow rate is 0.1 L/sec. “Duration” of the breath pulse is only 0.3 seconds. Breath air is modelled as an ideal gas. The maximum pressure at the exhalation portion inlet is 2.65 kPa as calculated from the CFD simulation. The filters are modelled as porous media. Representative values for solidity and the filter particle size are 0.3 and 0.3 m, respectively, while tortuosity is 0.8. The filters' effect on the air flow is also modelled and clearly shows that there is no capturing of particles in their volume and no interaction of virus particles with the filters. It was found that the virus particles bounce upon impact. The total simulation time was 0.15 sec up to the peak of the breath pulse. The SST k-ω turbulence model was used in the simulation for calculation of the particles' path. It is a two-equation eddy-viscosity model that is used for many aerodynamic applications, which combines the Wilcox k-omega and the k-ω models.

The estimated hit rate on the chip area in the above simulation is 4.54×10³ particles per second as calculated. This estimation is based on the total chip area and includes any rebounds that take place in the testing chamber over the chip surface. The design modification of creating a more torturous path has resulted in the particles spending more time on the impacting side of the chip.

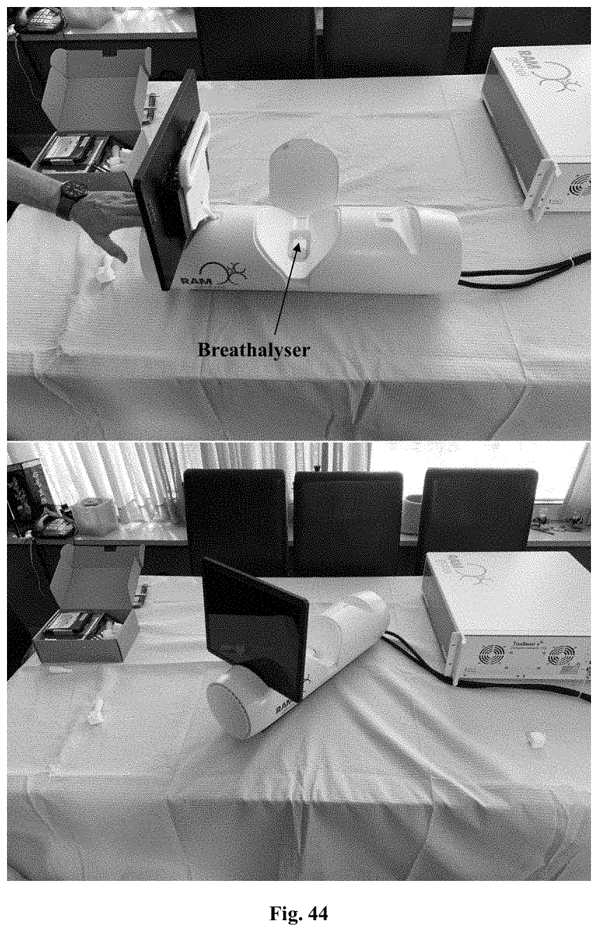

FIG. 44 shows the photographs of the marketing candidate of the breathalyser system including the breathalyser of the present invention and a miniaturised THz spectrometer custom-made and manufactured by the applicant.

In a particular embodiment, a method for label-free chemical detection and biomolecular diagnostics using the breathalyser of the present invention comprises:
(a) Blowing an air into the exhalation portion of the breathalyser of the present invention;
(b) Recording electrical signals received from the breathalyser over time at a resonance frequency in the THz frequency domain, said resonance frequency is dependent on inductance and capacitance of an analyte being tested in the sample and pre-selected based on a calibration of the sensor for said analyte;
(c) Transmitting the recorded signals from said breathalyser to an external memory for further processing; and
(d) Converting the transmitted signals to digital signals and processing the digital signals in the external memory in a form of frequency waveforms, comparing the recorded frequency waveforms with negative control waveforms stored in the external memory, and extracting chemical and biomolecular information from said waveforms in a form of readable data, thereby detecting and/or identifying a particular analyte in the sample.

In the above method, as explained above, each of said particular analytes being tested is characterised by a distinguished shift in the resonant frequency and by a unique fingerprint area in the recorded THz frequency waveform (spectrum). In a specific embodiment, the viral pathogen being tested is SARS-CoV-2.

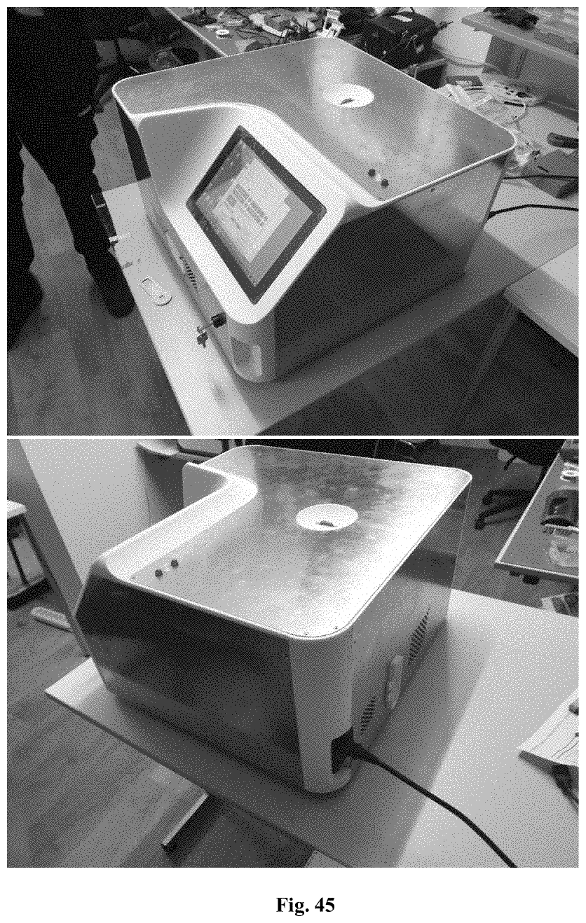

In some other embodiments, the microelectronic chip of the present invention is inserted in a laboratory THz spectrometer for laboratory measurements. FIG. 45 shows the photographs of the marketing candidate of the laboratory THz spectrometer custom-made by the applicant for laboratory measurements of samples deposited on the microelectronic chip of the present invention, including swab samples.

### Analytes, Transducers and Further Options

Upon applying the bias to the microelectronic sensor of the present invention, the nano-antenna structure resonates at a particular frequency in the THz frequency range, where the resonant frequency depends on inductance and capacitance of the structure. The THz frequency range is pre-selected such that it covers a resonant peak of the analyte to be detected. The analytes have a unique real and imaginary part of the refractive index and when they fall in a gap between the capacitor electrodes or in the nano-gap of metamaterial structures described above, the capacitance of the system changes so as the resonant frequency. Each and every analyte can be distinguished from each other by: a) finding the shift in resonant frequency that is unique to that particular analyte, b) taking a spectrum to identify the unique fingerprint in the THz spectrum of the particular analyte, and c) conducting a time series measurement to identify the changes in the resonant frequency. For the latter, the surface of the nanoantenna structure can be modified such that the analytes can be selectively bound, as described above. A time series measurement is suitable for studying the binding kinetics. Optionally, substrate of the microelectronic chip can be heated, so that the droplets are evaporated, increasing the chance of the analyte being tested to precipitate into the nano-gaps of the periodic structure. The sensor of the present invention can be made leak-proof for easy disposal.

Optionally, the surface of the nanoantenna periodic structure of the present invention can be modified with metallic nanoparticles, for example gold nanoparticles, to create plasmonic effect upon irradiation of the chip with excitation light.

Alternatively, an electro-optical crystal (EOC) transducer can be layered on the surface of the nanoantenna periodic structure of the present invention. The EOC may be any suitable electro-optical crystalline material such as LiNbO₃, which is brought into a contact with a medium to be sensed. The EOC is then illuminated with a polarised light. In case of the LiNbO₃ crystalline material, the wavelength of the polarised light is about 400-600 nm.

Modulated light from light source, such as such a surface-mounted-device light-emitting diode (SMD LED) or an UV-VIS-IR laser diode, which is further installed in the sensor, is suitable for illuminating the EOC layer, and then striking the nanoantenna periodic structure. The periodic structure is ultrasensitive to any smallest light intensity changes coming from the EOC transducer, thereby strongly affecting the structure capacitance and inductance. Depending on the excitation light wavelength, the position of the sensor relative to the incident light beam can be changed. For instance, in case of IR light (700-1500 nm), the sensor should be placed perpendicularly to the light beam for achieving the highest sensitivity. The parasitic charging of the EOC is compensated via the electrodes attached to the crystal. Additionally, a variety of light filters in front of the sensor can be utilised. Thus, the use of the plasmonic nanoparticles or additional EOC layer makes it possible to drastically increase sensitivity of the sensor.

In one embodiment, the microelectronic sensor of the present invention further comprises the following components:
(a) an μ-pulse generator for pulsed RF signal generation;
(b) an integrated DC-RF current amplifier or lock-in amplifier connected to said p-pulse generator for amplification of the signal obtained from said p-pulse generator;
(c) an analogue-to-digital converter (ADC) with in-built digital input/output card connected to the amplifier for converting the received analogue signal to a digital signal and outputting said digital signal to a microcontroller unit;
(d) the microcontroller unit (MCU) for processing and converting the received digital signal into data readable in a user interface or external memory; and
(e) a wireless connection module for wireless connection of said breathalyser to said user interface or external memory.

In another embodiment, the microelectronic sensor of the present invention further comprises:
(1) one or two out-input RFID-tag zero-power fractal antennas, each connected to the circuit, for RFID-tagging and further tracking a particular individual;
(2) a diode input-output separator to separate polarities in said circuit;
(3) an RFID integrated circuit for storing and processing signals received from said individual, and for modulating and demodulating radio-frequency (RF) signals, said RFID integrated circuit comprising:
   (a) a voltage source supplying electric current to said breathalyser and to said one or two RFID-tag zero-power fractal antennas;
   (b) an integrated or CMOS current amplifier for amplification of an electric current obtained from said breathalyser;
   (c) an analogue-to-digital converter (ADC) with wireless input/output modules connected to said current amplifier for wireless outputting the converted signal to a user interface or external memory;
   (d) a microcontroller unit (MCU) for processing and converting the received signal into data readable in said user interface or external memory; and
   (e) a wireless connection module for wireless connecting of said sensor to said user interface or external memory.

The ADC card may be any suitable analogue-to-digital converter data logger card that can be purchased, for example, from National Instruments® or LabJack®. In a specific embodiment, the wireless connection module may be a short-range Bluetooth® or NFC providing wireless communication between the sensor and the readout module for up to 20 m. If this connection module is Wi-Fi, the connection can be established with a network for up to 200 nm, while GSM allows the worldwide communication to a cloud. The external memory may be a mobile device (such as a smartphone), desktop computer, server, remote storage, internet storage or cloud.

In some embodiments, the nanoantenna periodic structure of the present invention further comprises at least one chemical or biomolecular layer immobilised on top of said nanoantenna periodic structure and capable of binding or adsorbing analytes being tested from the sample. The chemical or biomolecular layer allows sensing for example, gas molecules to be bound or adsorbed and then detected. This layer may further increase sensitivity and selectivity of the sensor based on the specific binding of the analytes. The chemical or biomolecular layer is composed, for example, of polymers, redox-active molecules, such as phthalocyanines, metalorganic frameworks, such as metal porphyrins, for example hemin, biomolecules, for example receptors, antibodies, DNA, aptamers or proteins, water molecules, for example forming a water vapour layer, such as a boundary surface water layer, oxides, semi-conductive layer or catalytic metallic layer. The layer is immobilised over either a portion of the nanoantenna periodic structure surface or substantially over its entire surface to further improve sensitivity of the sensor for detection of the analytes.

By using coatings with selective adsorption properties, breathalysers detecting specific chemical or biological compounds for both gas-phase and liquid-phase environments have now been developed by the present applicants. Typically, durable oxide-based coatings that are chemically modified to provide the required adsorption characteristics are used. These coatings can selectively adsorb ionic species from solution for use in applications such as monitoring electroplating processes or waste streams for toxic metals such as chromium, cadmium, or lead.

Polymer coatings that adsorb a wide variety of chemicals are ideally suited for monitoring the highly regulated ozone-depleting chlorinated hydrocarbons. Simultaneous measurement of the wave velocity and attenuation can be used to identify chemical compounds and their concentration. One of the applications of the sensors of the present invention is the selective detection of organophosphates, which are a common class of chemical warfare agent. The detection of these chemicals is done by the active chemical layer composed of thin films of self-assembled monolayers. The sensitivity of these films on the piezoelectric material of the sensor endows the sensor with immunity to interference from water vapour and common organic solvents while providing sensitivity in the part-per-billion concentration of organophosphates. As a result, arrays of such sensors with appropriate coatings can be used to detect the production of chemical weapons.

Another application of the sensors of the present invention is a chemical detection and analysis of environmentally toxic compounds and toxins, such as food toxins, for example aflatoxin, neurotoxic compounds, for example lead, methanol, manganese glutamate, nitrix oxide, Botox, tetanus toxin or tetrodotoxin, shellfish poisoning toxins, for example saxitoxin or microcystin, Bisphenol A, oxybenzone and butylated hydroxyanisole. In general, chemical detection and analysis of toxic compounds can be aimed at determining the level or activity of these compounds in the emission sample (into which the toxic compound is incorporated en route to human exposure, for example in industrial effluents), in the transport medium (for example, air, waste water, soil, skin, blood or urine), and at the point of human exposure, for example in potable water. Sensing the emission sample, the transport medium, and the point of human exposure may be necessary for a comprehensive plan designed both to detect toxic compounds, analyse them and to exert control on the emission of the toxic compounds in order to achieve hazard reduction.

For a given toxic analyte, chemical sensors of the present application will certainly differ in sensitivity, selectivity, or other characteristics, which may be required to monitor the emission sample, the transport medium, and individual exposure. Concentration of a toxic compound is typically greater in the emission sample than after dispersal in a transport medium and can vary widely. The physical and chemical properties of the analyte and its immediate environment (airborne vapour, contained in solid or liquid aerosol, chemically or photochemically reactive and decomposing into compounds of different toxicity, radioactive, ionic, acidic or lipophilic) are also influential in the design of a suitable configuration for the sensor of an embodiment.

Still another application of the sensors of the present invention is a chemical detection of explosives. In general, a large range of explosives can be detected with the sensor of an embodiment. A distinction is made between the bulk explosives and the trace explosives. In case of the trace explosives, the sensor is capable of detecting vapours of the explosive chemicals, thereby detecting the trace quantities emitted from explosive materials either directly in the environment or in the particulates of explosive materials that have been collected and then vaporised in the laboratory within the analytical instrument. The sensor of an embodiment can be operated both by direct sampling of the air containing the trace explosive vapours as well as by vaporising a sample that was collected by swiping a surface contaminated with explosive particulates.

Apart from simply being able to detect explosive materials, the sensor of the present invention is capable of identifying and quantifying the explosives. In general, a sensor that is used as a safety measure at airports will have other requirements than one that will be used in the field during military missions. Therefore, the configuration of the sensor can vary dependent on the particular application. There are different requirements to the throughput and, because of elevated background levels in military environments, the dynamic range. Furthermore, the military sensor for detection and analysis of explosives should be portable compared to the fixed sensors in laboratories or airports. Another consideration is the difference between detection and identification. In some instances, a device will be used to sense whether a certain explosive material is present, whereas in others it is also necessary to determine which explosive compound it is. Furthermore, it can be important to consider how many different compounds, or groups of compounds, one device must be able to detect or identify. Different sensor configurations described below meet the above requirements for different types of the sensors.

Instead of detecting the explosive compounds themselves, the sensors of the present invention may also be used to detect other materials that could indicate the presence of an explosive material. These “other” materials are actually associated compounds that tend to be present when explosives are present, such as decomposition gases or even taggants, materials that have been added during the production of the explosive to facilitate the detection. An advantage of this approach is that taggants and some associated compounds have a higher vapour pressure than the explosive compound itself and are thus easier to detect. In addition to the sensitivity, the selectivity of the sensor should also be considered. The selectivity of the sensors of an embodiment to vapours of the trace explosives may be increased by using them in an array. By using the sensors in an array, it is possible to obtain a signal similar to an artificial olfactory system of a nose when the responses of a number of sensors are combined to give a fingerprint-like signal. In this case, pattern recognition methods, such as multiple axes radar plots, can be used to analyse the signal, match it to known responses from a database, and thus identify the explosive.

Examples of the explosive materials detected by the sensors of the invention in aqueous medium are picrates, nitrates, trinitro derivatives, such as 2,4,6-trinitrotoluene (TNT), 1,3,5-trinitro-1,3,5-triazinane (RDX), N-methyl-N-(2,4,6-trinitrophenyl)nitramide (nitramine or tetryl), pentaerythritol tetranitrate (PETN), trinitroglycerine, nitric esters, derivatives of chloric and perchloric acids, azides, and various other compounds that can produce an explosion, such as fulminates, acetylides, and nitrogen rich compounds such as tetrazene, octahydro-1,3,5,7-tetranitro-1,3,5,7-tetrazocine (HMX), peroxides (such as triacetone trioxide), C4 plastic explosives and ozonides. In addition to the explosives, nitrobenzene, 2, 4-dinitrotoluene and several other organic compounds were tested being concomitant chemicals of TNT or some common water pollutants. The biomolecular layer (20) can be for example, a layer of the antibodies immobilised against a specific explosive compound. Alternatively, the molecular layer can be phthalocyanine system having 2,2,3,3-tetrafluoropropyloxy substituents or cyclodextrin as sensitive materials for the detection of different explosives in aqueous media, in particular nitro-containing organic compounds.

As mentioned above, a biomolecular layer sensitive to a certain target biomolecule, such as a specific pathogen, may be deposited on the surface of the nanoantenna periodic structure of the present invention. As a result, specific pathogens can be bound and reside in the nano-gaps to continuously transmit the signal, thereby increasing sensitivity and specificity of the sensor. For example, biological pathogens may be captured very selectively by the biomolecular layer consisting of specific biological receptor molecules, such as antibodies, short peptide chains or single-strand DNAs, which are capable of distinguishing between closely related pathogens. Thus, another application of the sensors of the present invention is a biomolecular diagnostic including detection of DNA and proteins. In that case, the biomolecular specific layer allows proteins and DNA molecules to be bound or adsorbed and then detected and identified. This biomolecular layer further increases the sensitivity and selectivity of the sensor. The biomolecular layer can be made of various capturing molecules, such as primary, secondary antibodies or fragments thereof against certain proteins to be detected, or their corresponding antigens, enzymes or their substrates, specific polynucleotide sequences complimentary to the DNA to be detected, aptamers, receptor proteins or molecularly imprinted polymers.

### EXAMPLES

### Example 1: Sensitivity of the Sensor Against SARS-CoV-2

In order to find the limit of detection of the sensor, measurements were taken on −ve and +ve samples with a series of CT values ranging from CT18 to CT38. CT values indicate the cycle threshold used in PCR for exponential amplification of the target specimen and is inversely related to the viral load in the sample. CT40 is the accepted minimum viral load that can be detected by high-end PCR techniques. The provided range of samples were analysed using the transmission ratio method developed by the inventors (the protocol of this method is available on request).

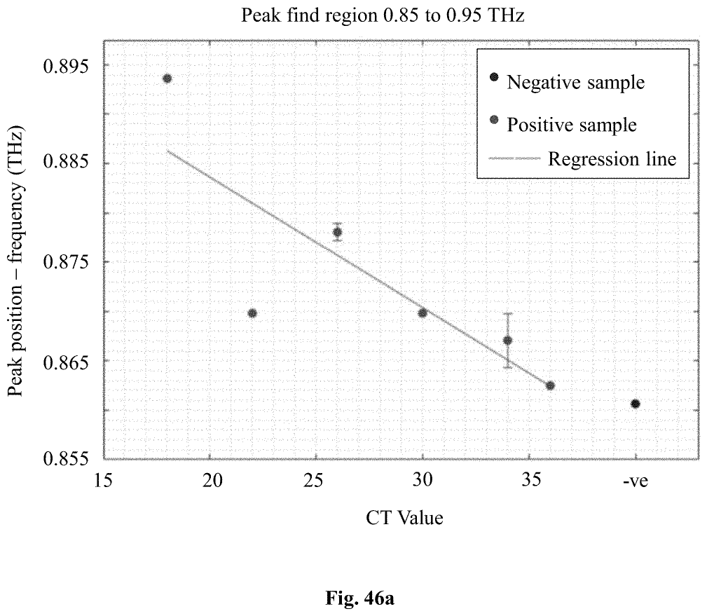

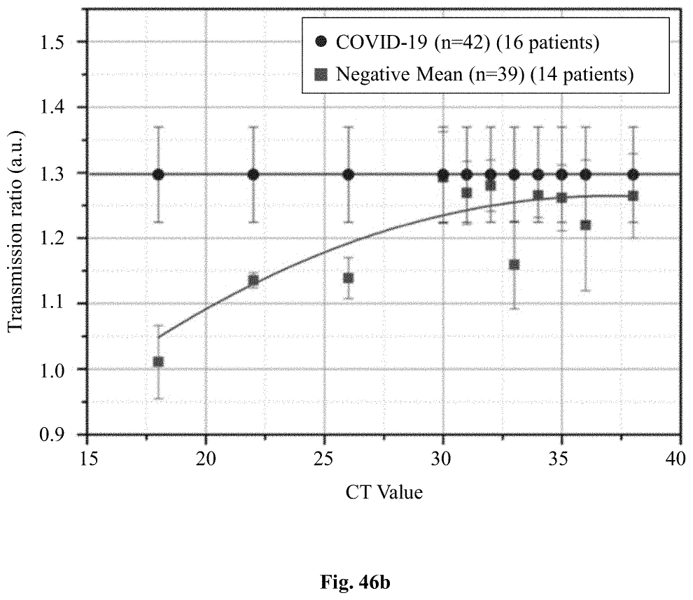

FIGS. 46a-46b show the relationship between absorption peaks and viral load (CT values) detected by transmission ratio method using nano absorber devices on quartz substrate. FIG. 46a shows the position of the absorption peak in the resonance region of our sensor for samples with different CT values. A linear trend is observed such that the peak position shifts to higher frequencies with increasing viral load. FIG. 46b shows the relative intensity of the peak amplitude against different CT values. The error bars represent the standard deviation over the entire sample set, including −ve samples shown for comparison. The −ve samples do not have an associated CT value and are included here only as a guide for comparison. The trend shows indicates sensitivity to viral load suggesting the limit of detection for our technique is around CT30. Beyond CT30 the transmission ratio falls within the standard deviation of −ve samples.

Thus, the data collected in a clinical study confirms that the sensor of the present invention can detect SARS-CoV-2 with 100% accuracy up to CT 30 and close to 90% accuracy up to CT 40 as seen from FIGS. 46a-46b. This is achieved from 7 μl of sample volume from a 1 ml solution. The sensor was able to detect SARS-CoV-2 from 0.7% of the solution, where the number of virus on the sensor surface is extremely low. Number of viruses per 5 μl volume of buffer increases by an order of magnitude for every 3 CT values. For example, CT30 corresponds to 10000 copies per 5 μl, whereas CT33 corresponds to 1000 copies per 5 μl. This would imply that the number of virus copies in exhalation air is considerably higher compared to the viral load that got dispensed on the sensor of the present invention.

In these experiments, 7 μl of solution was disperse over an area of 4×4 mm and dried. For a sample with CT30, (even though higher CT value can be detected), assuming there are 15,000 viruses per 7 μl, after drying over an area of 4×4 mm, the particle density will be 0.0009 viruses/μm². This is an extremely small number and absolutely nothing has been done to make sure that the virus particles fall in the gaps. The probability of viruses falling in a gap of 4 μm×300 nm is close to zero. The present experiment clearly demonstrates that even with this best-case scenario (CT 30, 100% accuracy), the sensor of the present invention is able to detect SARS-CoV-2 no matter whether the particles fall in the gap or not for the reasons explained in the description. As can be seen in this example, a very good quantitative agreement with the predicted shift and change for different CT values is obtained. The change to the dielectric constant due to change in concentration would result in amplitude change and not in the combined frequency plus amplitude change. This again shows that an additional mechanism is working here which is the contribution of the virus absorption and emission into the metamaterial creating this change.

### Example 2: Specificity of the Sensor Against SARS-CoV-2

To deploy the sensor of the present invention on a wider scale, it should be able to distinguish virus specimens from negative samples and other viruses with comparable viral loads, for example SARS-CoV-2 versus Corona 1 (HCoV-OC43), 2 (HCoV-HKU1) and 3 (HCoV-229E). To confirm this, a series of experiments was carried out with positive clinical samples of Corona 1, 2 and 3, SARS-CoV-2 (+ve) and −ve samples provided by a hospital.

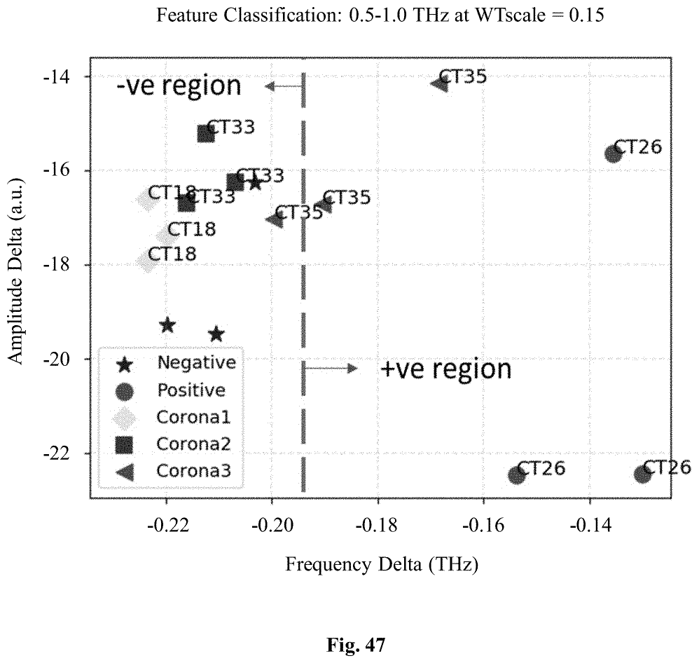

FIG. 47 shows the feature classification based on wavelet analysis of the THz spectra comparing the +ve samples for SARS-CoV-2, Corona 1, 2, 3 and −ve samples. CT values are annotated for the +ve samples, showing a clear distinction between SARS-CoV-2 and other Corona viruses as well as −ve samples. The dotted line in the plot is shown here as a guide for comparison. These data show that SARS-CoV-2+ve samples can be distinguished from other viruses and −ve samples.

As clearly seen from FIG. 47, the SARS-CoV-2 sample stands out, while the others viruses, which are the same size as SARS-CoV-2 and have the same or similar dielectric constant, to SARS-CoV-2 are clustered together in the same region. If the sensing mechanism would be a purely gap-based sensing of dielectric constant, there should be four different clusters observed in the same area. However, this is not the case, because the sensor of the present invention is specifically designed for a particular virus (SARS-CoV-2 in the present case) and is not dependent on the virus falling in the gap. As explained above throughout the description, this is very different from the prior art, such as Ji-Hun Kang et al. (2018), where they measure the effective dielectric constant in the gap.

Moreover, the negative samples are falling in the same region as the other virus samples. However, the SARS-CoV-2 virus is falling in a different region. This result is extremely important, because it shows that when using a dirty sample, one would not be able to detect the difference between a positive and negative sample, if only using dielectric constant measurements.

Furthermore, FIG. 47 shows that HCoV-OC43 and HCoV-HKU1 are clustered together and are away from the cluster of SARS-CoV-2. HCoV-OC43 and HCoV-HKU1 have very similar structures, dielectric constants and dimensions. This proves that there is an amplification of the interaction between the SARS-CoV-2 virus and the metamolecules at a particular frequency and is different from sensing the dielectric constant. As shown in the present description, this frequency was found by the present inventors via a systematic study. If another virus has to be detected, all that should be done is to find the frequency where it interacts better with THz radiation and tune the frequency very close to that frequency.

To conclude, viruses are charged particles. Therefore, they interact very well at certain frequency bands in the THz frequency range. Metamaterials and metasurfaces effectively absorb the THz radiation at certain frequencies creating the resonance. This is an overall effect and not just confined to what is happening in the gap. The metamolecules, the structure itself, the gap, the adjacent structures, substrate—all of them play a role in this resonance. When there is a virus which resides on top of this resonating structure, it can disturb this resonance. The near-field interaction is what enables the detection.

### Example 3: Reproducibility of the Sensor Against SARS-CoV-2

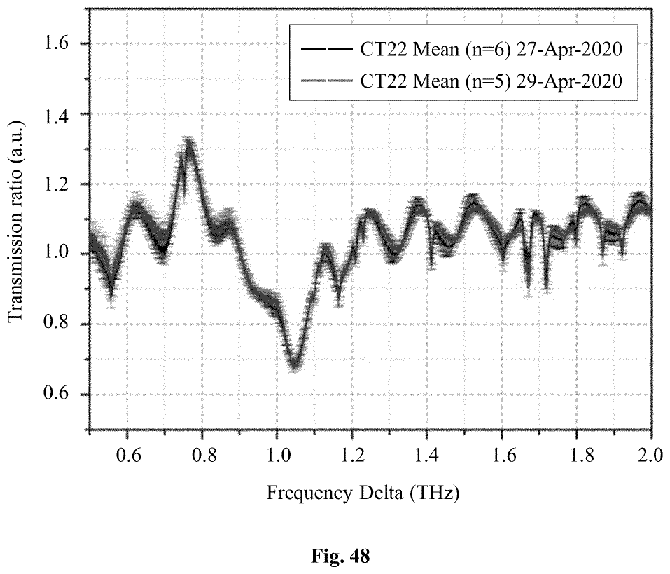

Another important aspect for the test of the sensor is the repeatability of measurements over different days. For this test, frozen +ve solution was aliquoted and each portion was measured over different days. The transmission ratios are shown in FIG. 48. The observations indicate that measurements are indeed repeatable (reproducible) over different days demonstrating a consistency in the measurements. The small variability comes from the environmental and sample preparation conditions.

### Example 4: Accuracy of the Sensor Against SARS-CoV-2

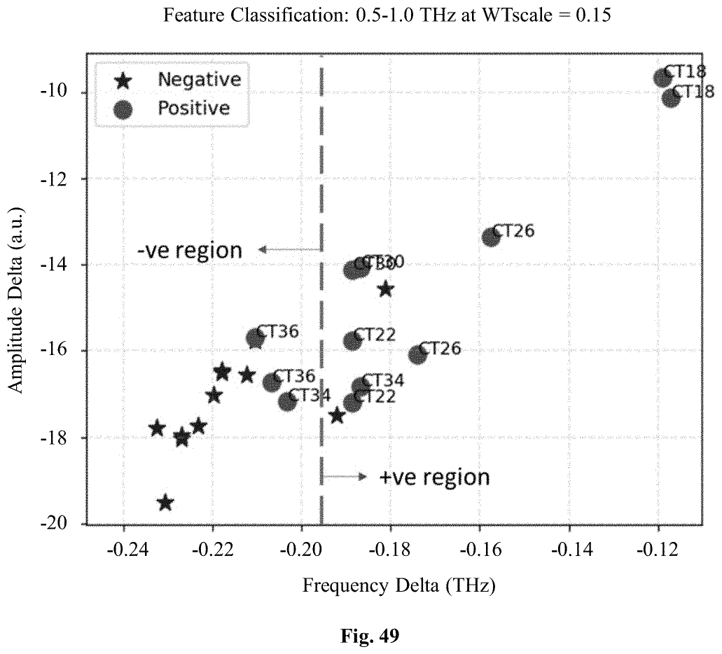

One of the main characteristics of any sensor is its accuracy. The present sensor has been tested for accuracy in identifying +ve and −ve samples, within its limit of detection. In order to check if the +ve and −ve samples can be predicted accurately, a scatter plot shown in FIG. 49 was generated using the above discussed wavelet analysis. A frequency delta of zero would imply that both sample and reference are identical to each other.

As it can be seen in FIG. 49, +ve samples (red dots) have a lower frequency delta compared to −ve samples. The higher the viral load, the smaller its corresponding ordinate on this plot. Ideally, a large separation and clustering between +ve and −ve samples would be expected, so they can be distinguished unambiguously. However, in real world, there is a spread in the data, creating an overlap that depends on the sensitivity of the sensor. In FIG. 49, if the area to the right of the arbitrarily drawn dashed line is considered as +ve and the area to the left as −ve, the accuracy can then be estimates as approximately 80%, including two false +ve results. A few false +ve results are less concerning than many false −ve results, which is actually the case here.

Moreover, most of the present classification errors occur for samples with relatively low viral load (≥CT34). A prediction accuracy of the present AI-based analysis was also 75% despite the relatively small dataset. The present wavelet analysis using this dataset contained all available viral loads, and it was possible to observe a separation in the positive and negative sample data. To follow through, variability in the present data was analysed from several days of experiments and identified key factors discussed below were considered in order to increase the accuracy. By correcting for these factors, the accuracy can be brought closer to 100% over a broad range of viral loads.

### Example 5: Effect of Dilution

Unlike PCR techniques, the sensor and the method of the present invention do not involve any kind of sample amplification and relies solely on the physical presence of the target analyte on the active areas on the sensor. This is confirmed by results shown above regarding sensitivity and accuracy of the present sensor, where the analysis showed better classification of samples with higher viral loads. It turns out that higher numbers of virus copies obtained, for example, from a collected swab would improve the limit of detection of the sensor.

The samples provided for this study were swab solutions collected for PCR where the swab sample had been diluted in 1 ml buffer solution, of which only 7 μl was pipetted on the sensor for testing. This accounts for only 0.7% of the entire available volume. Hence, it can be assumed that the number of viruses on the sensor surface is proportionally lower compared to the viral load obtained from the patient. As discussed in previous sections, the present sensor was able to classify samples down to CT30 with reasonable accuracy despite this limitation.

Number of viruses per 5 μl volume of buffer increases by an order of magnitude for every 3 (three) CT values. For example, CT33 corresponds to 1000 copies per 5 μl of the sample, whereas CT36 corresponds to 100 copies per 5 μl. This would imply that the number of virus copies on the swab is considerably higher compared to the viral load that got dispensed on the sensor. As a very rough estimate, if the dilution volume is reduced from 1 ml to 10 μl, a hundred-fold increase in virus copies per unit volume of buffer can be expected. This would increase the limit of detection correspondingly by approximately 6 (six) CT values.

As mentioned above, raw samples used in the present invention use very little or no dilution media. Therefore, concentrated swab samples were used for testing in the present example. By employing this approach, it should be possible to increase the sensor limit of detection by three to six (3-6) CT values. To test this hypothesis, 7 μl +ve solution was pipetted with the CT27 sample several times with drying steps between each sample deposition. Between intermediate deposition steps, the THz spectra were taken for further analysis of sensitivity vs concentration. This process was repeated three times.

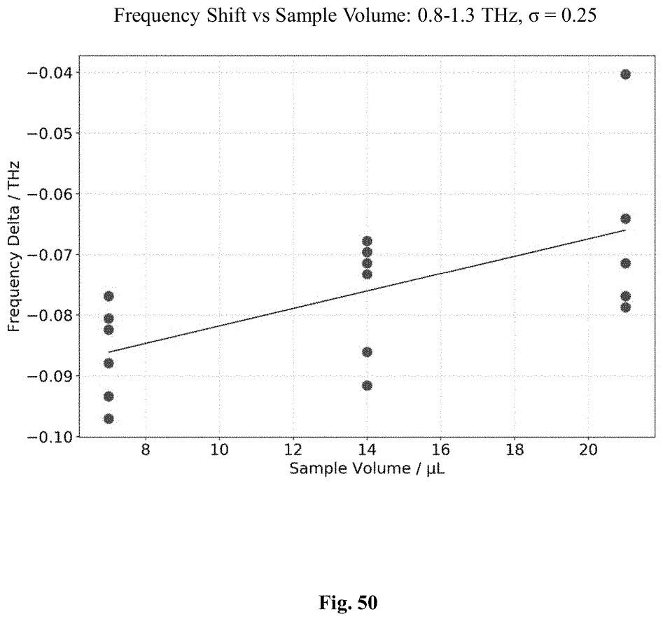

The results of this experiment are presented in FIG. 50 which is the wavelet analysis showing that as the number of layers (number of pipetting rounds) increases, the frequency delta reduces and moves closer to zero. This result is consistent with previously observed trends of frequency delta vs increasing viral loads in Example 4 (see FIG. 49) above. From Example 4, it was observed that higher viral loads (lower CT values) produce smaller frequency deltas, approaching zero (see FIG. 50). A similar trend is also observed by increasing the thickness of the analyte deposited layer. Therefore, it can be concluded that reducing the dilution volume used in sample preparation will considerably improve the sensor's limit of detection. This approach involves a new sample collection method that would allow concentrated solution to be applied directly onto the sensor. One of the embodiments of the present invention describes the breathalyser which solves this problem by collecting the raw samples directly from breath onto the chip surface without any dilution and pre-treatment. The breathalyser is therefore aimed for any quick spot checks outside the lab, for example at transport hubs, for point-of-care diagnostics and for home use.

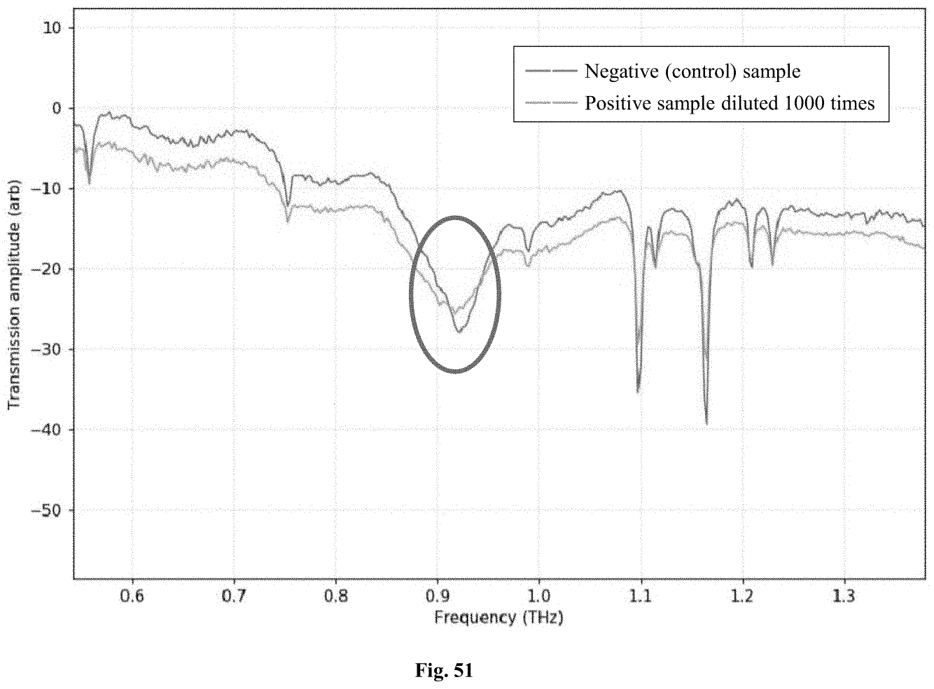

FIG. 51 shows the control sensor of the present invention (negative sample) (blue line) and the sensor of the present invention with the 1000-times diluted positive sample (red line). As can be seen here, the sensor of the present invention capable of creating the SPP resonance clearly shows the difference between the positive and negative samples, where the positive sample was diluted by 1000 times, but still exhibits the change is a frequency (phase shift). Unlike the prior art sensor, this sensor of the present invention enables a frequency shift at the absorption and emission frequency of the virus even at ultra-low concentrations of the virus and when the resonance frequency is set to the “on-resonance” frequency of the virus. The shift is completely predictable.

1. A microelectronic sensor for non-invasive and label-free chemical detection and biomolecular diagnostics of analytes in a raw sample, comprising a microelectronic chip and a sample collection system attached to said microelectronic chip or incorporating said microelectronic chip, said sample collection system is suitable for sample collection of a raw sample taken directly from a subject being tested without any purification and without any chemical or biological separation, and for delivery of the sample to said microelectronic chip, wherein said microelectronic chip comprises:
(a) a nanoantenna structure, said nanoantenna structure is arranged in a periodic array of metamolecules and configured to detect and transmit signals through said sample in a terahertz (THz) frequency range; and
(b) an integrated circuit for storing and processing signals in a THz frequency domain, and for modulating and demodulating radio-frequency (RF) signals;
characterised in that each of said metamolecules in the array is composed of at least one split-ring resonator and a wave container or a wave bouncer, said wave container confines and said wave bouncer bounces electromagnetic waves received from said at least one split-ring resonator, both the wave container and the wave bouncer are designed to excite a dark mode in said at least one split-ring resonator, followed by coupling the excited dark mode back into said at least one split-ring resonator.

2. The microelectronic sensor of claim 1, wherein said at least one split-ring resonator is composed of a metal square-, round-, rectangular-, hexagonal-, spiral- or any other shaped ring (wire) having at least one split (gap) in the ring and suitable for resonating in the THz frequency range.

3. The microelectronic sensor of claim 1, wherein said at least one split-ring resonator is asymmetric.

4. The microelectronic sensor of claim 1, wherein said at least one split-ring resonator has a geometry selected from a rod split-ring, round split-ring, square-split ring, rectangular split-ring, hexagonal split-ring, nested split-ring, single split-ring, split-ring having more than one split (gap) in the ring, deformed split-ring, spiral split-ring and spiral resonator suitable for resonating in the THz frequency range.

5. The microelectronic sensor of claim 1, wherein said wave container is selected from a metal ring, metal square, metal rectangle, metal hexagon and any other shape or array thereof suitable for confining electromagnetic waves received from said at least one split-ring resonator, said wave container is designed to excite a dark mode in said at least one split-ring resonator, followed by coupling the excited dark mode back into said at least one split-ring resonator.

6. The microelectronic sensor of claim 1, wherein said wave bouncer is selected from a metal bar, metal segment or any other metal fragment or array thereof suitable for bouncing electromagnetic waves received from said at least one split-ring resonator, said wave bouncer is designed to excite a dark mode in said at least one split-ring resonator, followed by coupling the excited dark mode back into said at least one split-ring resonator.

7. The microelectronic sensor of claim 1, wherein each said metamolecule is composed of:
(a) two square-shape split-ring resonators and a single metal bar over the resonators, said metal bar is designed to excite a dark mode in said resonators, followed by coupling it into the resonators; or
(b) a spiral-shape resonator and a metal ring wave container surrounding and confining said spiral-shape resonator, said metal ring is designed to excite a dark mode in the spiral-shape resonator, followed by coupling it into the said spiral-shape resonator; or
(c) a round-shape split-ring resonator having at least two splits in the ring and a metal bar under said round-shape split-ring resonator, said metal bar is designed to excite a dark mode in the round-shape split-ring resonator, followed by coupling it into said split-ring resonator; or
(d) an inner hexagon-shape split-ring resonator having at least one split in the ring and six outer metal hexagons surrounding said hexagon-shape split-ring resonator, said six outer metal hexagons form the wave container designed to excite a dark mode in the inner hexagon-shape split-ring resonator, followed by coupling it into said inner hexagon-shape split-ring resonator; or
(e) a round-shape split-ring resonator having at least two splits in the ring and a metal square-shaped wave container, said metal square-shaped wave container is designed to excite a dark mode in the round-shape split-ring resonator, followed by coupling it into said round-shape split-ring resonator.

8-11. (canceled)

12. The microelectronic sensor of claim 7, wherein walls of said metal square-shaped wave container are symmetrically split to form additional resonance structures creating bright mode.

13. The microelectronic sensor of claim 1, further comprising at least one index-matching polymeric layer applied on one side or on both sides of the nanoantenna structure and designed to reduce internal reflections.

14. The microelectronic sensor of claim 13, wherein said polymeric layer is composed of polymethyl methacrylate (PMMA) polymer.

15. The microelectronic sensor of claim 13, further comprising and adhesive layer.

16. (canceled)

17. The microelectronic sensor of claim 1, wherein said nanoantenna periodic structure is composed of gold, gold/chromium, gold/doped silicon/silver or other similar metal periodic structures, or metamaterials designed to modulate propagation of THz electromagnetic waves in desired directions.

18. (canceled)

19. The microelectronic sensor of claim 17, wherein said metamaterials are graphene, graphene/gold or copper/single layer graphene/copper composite.

20. The microelectronic sensor of claim 1, wherein said nanoantenna periodic structure further comprises metallic nanoparticles, such as gold nanoparticles deposited on said periodic structure, to create plasmonic effects upon irradiation of the structure with excitation light, or an electro-optical crystal (EOC) transducer layer, such as LiNbO₃, deposited on said periodic structure and designed to be brought into a contact with the sample and illuminated with a polarised light, thereby making it suitable to modulate the structure capacitance and inductance, and increase sensitivity of the sensor.

21-24. (canceled)

25. The microelectronic sensor of claim 1, wherein the sample collection system is a sampling swab attached to the microelectronic chip, or a breathalyser tube incorporating the microelectronic chip, or wherein said microelectronic senor is inserted in a laboratory THz spectrometer for laboratory measurements.

26-30. (canceled)

31. A breathalyser for non-invasive and label-free chemical detection and biomolecular diagnostics of raw breath sample received directly from a subject being tested without any substantive purification and without any chemical or biological separation, comprising:
an integrated tube having an exhalation portion with an inlet (air intake) area and an exhaust portion with an outlet (focusing) area, said tube being placed in a housing transparent to terahertz radiation and suitable for collecting a sample of exhalation air and transferring said sample to a testing chamber;
the testing chamber integrated inside said housing, attached to said exhalation portion and designed to provide housing for an integrated circuit, battery and other electronic components, and to receive, filter and analyse said sample, said testing chamber comprises at least one filter suitable for filtering the sample and the integrated microelectronic sensor of claim 1; and
an integrated circuit for storing and processing signals in a THz (terahertz) frequency domain, and for modulating and demodulating radio-frequency (RF) signals.

32. A method for label-free chemical detection and biomolecular diagnostics comprises:
(a) Blowing an air into the exhalation portion of the breathalyser of claim 31;
(b) Recording electrical signals received from the breathalyser over time at a resonance frequency in the THz frequency domain, said resonance frequency is dependent on inductance and capacitance of an analyte being tested in the sample and pre-selected based on a calibration of the sensor for said analyte;
(c) Transmitting the recorded signals from said breathalyser to an external memory for further processing; and
(d) Converting the transmitted signals to digital signals and processing the digital signals in the external memory in a form of frequency waveforms, comparing the recorded frequency waveforms with negative control waveforms stored in the external memory, and extracting chemical and biomolecular information from said waveforms in a form of readable data, thereby detecting and/or identifying a particular analyte in the blown air.

33. The method of claim 32, wherein each of said analytes being tested is characterised by a distinguished shift in a THz resonant frequency and by a unique fingerprint area in the recorded frequency waveform.

34. The method of claim 32, wherein said analyte is selected from the group of:
toxic metals, such as chromium, cadmium or lead,
regulated ozone-depleting chlorinated hydrocarbons,
food toxins, such as aflatoxin, and shellfish poisoning toxins, such as saxitoxin or microcystin,
neurotoxic compounds, such as methanol, manganese glutamate, nitrix oxide, tetanus toxin or tetrodotoxin, Botox, oxybenzone, Bisphenol A, or butylated hydroxyanisole,
explosives, such as picrates, nitrates, trinitro derivatives, such as 2,4,6-trinitrotoluene (TNT), 1,3,5-trinitro-1,3,5-triazinane (RDX), trinitroglycerine, N-methyl-N-(2,4,6-trinitrophenyl)nitramide (nitramine or tetryl), pentaerythritol tetranitrate (PETN), nitric ester, azide, derivates of chloric and perchloric acids, fulminate, acetylide, and nitrogen rich compounds, such as tetrazene, octahydro-1,3,5,7-tetranitro-1,3,5,7-tetrazocine (HMX), peroxide, such as triacetone trioxide, C4 plastic explosive and ozonidesor, or an associated compound of said explosives, such as a decomposition gases or taggants, and
biological pathogens, such as a respiratory viral or bacterial pathogen, an airborne pathogen, a plant pathogen, a pathogen from infected animals or a human viral pathogen.

35. The method of claim 34, wherein said viral pathogen is SARS-CoV-2.
